(19) United States
(12) Patent Application Publication
(10) Pub. No.: US 2019/0315731 A1
(43) Pub. Date: Oct. 17, 2019

(54) 6-(BUTA-1,3-DIYN-1-YL)BENZO[D]THIAZOLE DERIVATIVES

(71) Applicants: Jörg Volker, Allschwil BL (CH); Ralf Koberstein, Allschwil BL (CH); Idorsia Pharmaceuticals Ltd., Allschwil (CH)

(72) Inventors: Stefan DIETHELM, Allschwil (CH); Azely MIRRE, Allschwil (CH); Philippe PANCHAUD, Allschwil (CH); Christine SCHMITT, Allschwil (CH); Jean-Luc SPECKLIN, Allschwil (CH); Jean-Philippe SURIVET, Allschwil (CH)

(21) Appl. No.: 16/303,019

(22) PCT Filed: May 16, 2017
(86) PCT No.: PCT/EP2017/061689

§ 371 Date: Nov. 19, 2018

(30) Foreign Application Priority Data
May 17, 2016 (EP) PCT/EP2016/061039

(51) Int. Cl. C07D 417/12; A61P 31/04

(52) U.S. Cl. CPC C07D 417/12; A61P 31/04

(57) ABSTRACT

The invention relates to antibacterial compounds of formula I

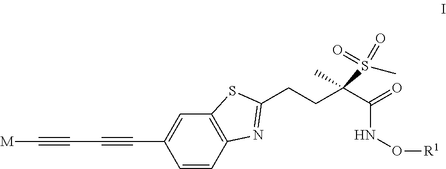

wherein the group M and R¹ are as defined in the claims, and salts thereof.

The present invention concerns 6-(buta-1,3-diyn-1-yl)benzo[d]thiazole derivatives, pharmaceutical compositions containing them and uses of these compounds in the manufacture of medicaments for the treatment of bacterial infections. These compounds are useful antimicrobial agents effective against a variety of human and veterinary pathogens, especially Gram-negative aerobic and anaerobic bacteria. The compounds of the present invention can optionally be employed in combination, either sequentially or simultaneously, with one or more therapeutic agents effective against bacterial infections.

The intensive use of antibiotics has exerted a selective evolutionary pressure on microorganisms to produce genetically based resistance mechanisms. Modern medicine and socio-economic behaviour exacerbate the problem of resistance development by creating slow growth situations for pathogenic microbes, e.g. in artificial joints, and by supporting long-term host reservoirs, e.g. in immune-compromised patients.

In hospital settings, an increasing number of strains of Staphylococcus aureus, Streptococcus pneumoniae, Enterococcus spp., Enterobacteriaceae such as Klebsiella pneumoniae, Acinetobacter baumannii and Pseudomonas aeruginosa, major sources of infections, are becoming multi-drug resistant and therefore difficult if not impossible to treat. This is particularly the case for Gram-negative organisms where the situation is getting worrisome since no novel agents have been approved for decades and the development pipeline looks empty.

Therefore, there is an important medical need for new antibacterial compounds addressing Gram-negative resistant bacteria, in particular third generation cephalosporins- and carbapenem-resistant Klebsiella pneumoniae and multi-drug-resistant Pseudomonas aeruginosa and Acinetobacter baumannii. One way to tackle the problem of cross resistance to established classes of antibiotics is to inhibit a new essential target. In this respect, LpxC, which is an enzyme in the biosynthesis of lipopolysaccharides (a major constituent of the outer membrane of Gram-negative bacteria), has received some attention and several patent applications relating to LpxC inhibitors have been published recently.

For example, WO 2011/045703, WO 2011/073845, WO 2012/120397, WO 2012/137094, WO 2012/137099 all describe antibacterial compounds based on a monocyclic carbocyclic or heterocyclic ring with a 4-(hydroxyamino)-3-methyl-3-(methylsulfonyl)-4-oxobutyl side chain (or an equivalent thereof).

WO 2013/170165 describes notably antibacterial compounds of formula (A1)

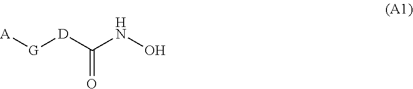

wherein A is a substituted alkyl group, wherein at least one substituent is hydroxy, or A is a substituted cycloalkyl group, wherein at least one substituent is hydroxy or hydroxyalkyl; G is a group comprising at least one carbon-carbon double or triple bond and/or a phenyl ring; D represents a group selected from

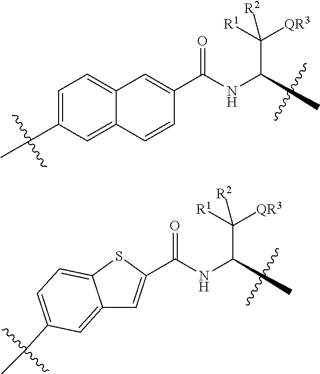

Q is O or NR, wherein R is H or an unsubstituted (C₁-C₃)alkyl; R¹ and R² independently are selected from the group consisting of H and substituted or unsubstituted (C₁-C₃)alkyl, or R¹ and R², together with the carbon atom to which they are attached, form an unsubstituted (C₃-C₄)cycloalkyl group or an unsubstituted 4-6 membered heterocyclic group; and R³ is selected from the group consisting of hydrogen, substituted or unsubstituted (C₁-C₃)alkyl, substituted or unsubstituted cycloalkyl, substituted or unsubstituted cycloalkylalkyl, substituted or unsubstituted aryl, substituted or unsubstituted arylalkyl, substituted or unsubstituted heterocyclyl, substituted or unsubstituted heterocyclylalkyl, substituted or unsubstituted heteroaryl, and substituted or unsubstituted heteroarylalkyl.

In WO 2015/036964, we have reported antibacterial 2H-indazole derivatives of general formula (A2)

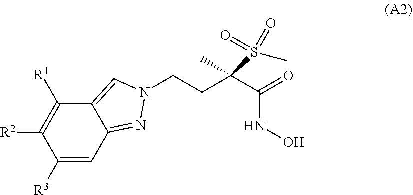

wherein

R¹ is H or halogen; R² is (C₃-C₄)alkynyloxy or the group M; R³ is H or halogen; M is notably the group M^{B} represented below

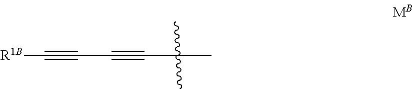

wherein R^{1B} is 3-hydroxyoxetan-3-yl, 3-hydroxythietan-3-yl, hydroxyalkyl, aminoalkyl, trans-2-hydroxymethyl-cycloprop-1-yl or 4-hydroxytetrahydro-2H-pyran-4-yl.

In WO 2015/091741, we have reported antibacterial 1H-indazole derivatives of general formula (A3)

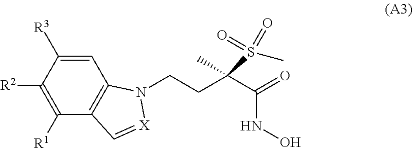

wherein X is N or CH; R¹ is H or halogen; R² is (C₃-C₄)alkynyloxy or the group M; R³ is H or halogen; M is notably the group M^{B} represented below

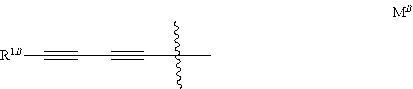

wherein R^{1B} is 3-hydroxyoxetan-3-yl, 3-hydroxythietan-3-yl, hydroxy(C₁-C₃)alkyl, amino(C₁-C₃)alkyl, 1-hydroxymethyl-cycloprop-1-yl or trans-2-hydroxymethyl-cycloprop-1-yl.

In WO 2015/132228, we have reported antibacterial 1,2-dihydro-3H-pyrrolo[1,2-c]imidazol-3-one derivatives of general formula (A4)

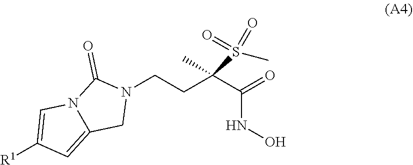

wherein R¹ is the group M; M is notably the group M^{B} represented below

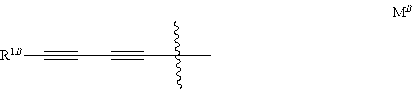

wherein R^{1B} is 3-hydroxyoxetan-3-yl, 3-hydroxythietan-3-yl, 3-(hydroxy(C₁-C₃)alkyl)oxetan-3-yl, hydroxy(C₁-C₃)alkyl, 1,2-dihydroxyethyl, amino(C₁-C₃)alkyl, 1-hydroxymethyl-cycloprop-1-yl, trans-2-hydroxymethyl-cycloprop-1-yl, trans-(cis-3,4-dihydroxy)-cyclopent-1-yl or 3-hydroxymethylbicyclo[1,1,1]pentan-1-yl.

In WO 2015/173329, we have reported antibacterial quinazoline-4(3H)-one derivatives of general formula (A5)

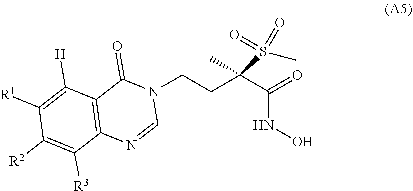

wherein R¹ is H or halogen; R² is the group M; R³ is H or halogen; M is notably the group M^{B} represented below

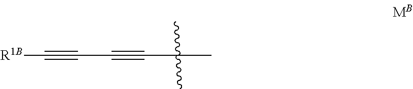

wherein R^{1B} is hydroxy(C₁-C₃)alkyl, amino(C₁-C₃)alkyl, 1,2-dihydroxyprop-3-yl, 1-amino-cycloprop-1-yl, 1-hydroxymethyl-cycloprop-1-yl, trans-2-hydroxymethyl-cycloprop-1-yl, trans-2-aminomethyl-cycloprop-1-yl, trans-2-hydroxymethyl-1-methyl-cycloprop-1-yl, trans-2-hydroxymethyl-2-methyl-cycloprop-1-yl, 1-(1,2-dihydroxyethyl)-cycloprop-1-yl, trans-2-(1,2-dihydroxyethyl)-cycloprop-1-yl, 3-hydroxyoxetan-3-yl, 3-(hydroxy(C₁-C₃)alkyl)oxetan-3-yl, 3-hydroxythietan-3-yl, trans-(cis-3,4-dihydroxy)-cyclopent-1-yl, 3-(2-aminoacetamido)cyclopentyl or 3-hydroxymethylbicyclo[1,1,1]pentan-1-yl.

In a further previous, yet unpublished patent application, we have reported antibacterial benzothiazole derivatives of general formula (A6)

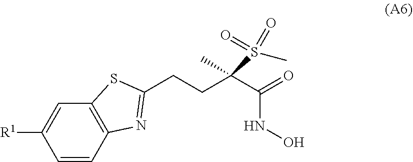

wherein

R¹ is the group M, whereby M is notably the group M^{B} represented below

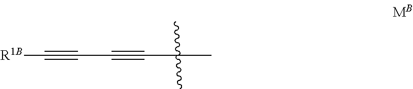

wherein R^{1B} is hydroxy(C₁-C₄)alkyl, dihydroxy(C₂-C₄)alkyl, amino(C₁-C₄)alkyl, di(C₁-C₄)alkylamino(C₁-C₃)alkyl, 1-amino-cycloprop-1-yl, 1-hydroxymethyl-cycloprop-1-yl, trans-2-hydroxymethyl-cycloprop-1-yl, trans-2-aminomethyl-cycloprop-1-yl, trans-2-hydroxymethyl-1-methyl-cycloprop-1-yl, trans-2-hydroxymethyl-2-methyl-cycloprop-1-yl, cis-1-fluoro-2-(hydroxymethyl)cycloprop-1-yl, cis-2-fluoro-2-(hydroxymethyl)cycloprop-1-yl, 2-(1,2-dihydroxyethyl)-cycloprop-1-yl, 1-(hydroxymethyl)-cyclobutan-1-yl, cis-3-(hydroxymethyl)-1-hydroxy-cyclobutan-1-yl, 3-hydroxyoxetan-3-yl, 3-hydroxyoxetan-3-yl-(C₁-C₃)alkyl, 3-aminooxetan-3-yl, 3-hydroxymethyl-oxetan-3-yl, trans-(cis-3,4-dihydroxy)-cyclopent-1-yl, 3-hydroxymethylbicyclo[1,1,1]pentan-1-yl, 4-hydroxytetrahydro-2H-pyran-4-yl, (3R,6S)-3-aminotetrahydro-2H-pyran-6-yl, piperidin-4-yl, 1-(2-hydroxyacetyl)piperidin-4-yl, 3-hydroxythietan-3-yl, 1-(2-hydroxyacetyl)azetidin-3-yl or 1-glycylazetidin-3-yl.

In WO 2011/073845, WO 2012/120397 or WO 2013/170165, further LpxC inhibitors are disclosed, among others the compounds of general formula (A7)

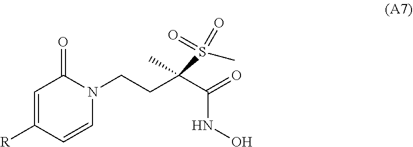

wherein R can notably be phenylethynyl or styryl.

Besides, in Montgomery et al., J. Med. Chem. (2012), 55(4), 1662-1670, yet further LpxC inhibitors are disclosed, among others the compound of formula (A8)

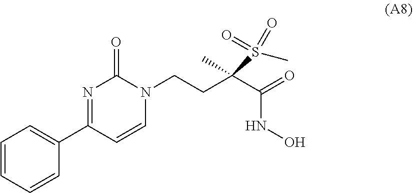 [SMILES: C[C@@](CCn1ccc(-c2ccccc2)nc1=O)(C(=O)NO)S(C)(=O)=O]

The instant invention provides new antibacterial 6-(buta-1,3-diyn-1-yl)benzo[d]thiazole derivatives, namely the compounds of formula I described herein.

Various embodiments of the invention are presented hereafter:

1) In a first embodiment, the invention relates to compounds of formula I

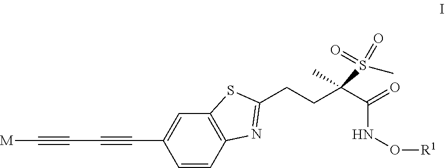

wherein

M is the group (4-hydroxypiperidin-1-yl)carbonyloxymethyl, (3-hydroxyazetidin-1-yl)carbonyloxymethyl or 1-(methylamino)cyclopropyl, or M represents one of the groups M^{A}, M^{B}, M^{C}, M^{D}, M^{E} and M^{F} represented below

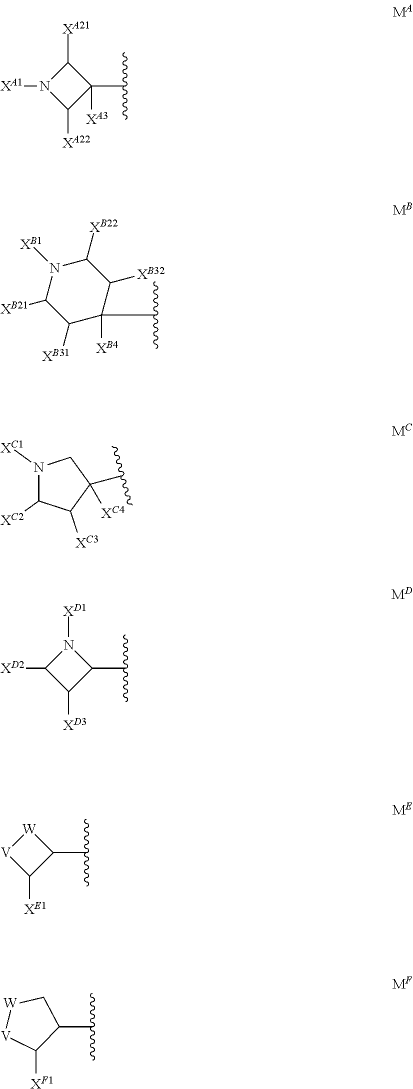

wherein

X^{A1} represents methyl-d, methyl-d2, (C₁-C₄)alkyl, ω-(C₂-C₃)haloalkyl, ω-hydroxy(C₂-C₄)alkyl, 2,3-dihydroxyprop-1-yl, 3-hydroxy-2-(hydroxymethyl)prop-1-yl, oxetan-3-yl, (oxetan-3-yl)methyl, thietan-3-yl, 1,1-dioxidothietan-3-yl, (C₃-C₆)cycloalkyl, 3-hydroxycyclobut-1-yl, 3-(ω-hydroxy(C₁-C₃)alkyl)cyclobut-1-yl, tetrahydropyran-4-yl, (C₃-C₆)cycloalkyl(C₁-C₃)alkyl or ω-phosphonooxy-(C₂-C₄)alkyl;

X^{A21} and X^{A22} each independently represent H, (C₁-C₄)alkyl, (C₁-C₃)haloalkyl or hydroxy(C₁-C₃)alkyl;

X^{A3} represents H, (C₁-C₃)alkyl or halogen;

X^{B1} represents (C₁-C₄)alkyl, ω-hydroxy(C₂-C₃)alkyl, (C₃-C₆)cycloalkyl, oxetan-3-yl or tetrahydropyran-4-yl;

X^{B21} and X^{B22} each independently represent H, (C₁-C₄)alkyl, (C₁-C₃)haloalkyl or hydroxy(C₁-C₃)alkyl;

X^{B31} and X^{B32} each independently represent H, halogen, hydroxy, (C₁-C₄)alkyl, (C₁-C₃)haloalkyl or hydroxy(C₁-C₃)alkyl;

X^{B4} represents H, halogen, hydroxy or (C₁-C₃)alkyl;

X^{C1} represents H, (C₁-C₄)alkyl, (C₃-C₆)cycloalkyl, ω-hydroxy(C₂-C₃)alkyl, oxetan-3-yl or tetrahydropyran-4-yl;

X^{C2} represents H, (C₁-C₄)alkyl, (C₁-C₃)haloalkyl or hydroxy(C₁-C₃)alkyl;

X^{C3} represents H, halogen (especially fluorine), hydroxy, (C₁-C₄)alkyl, (C₁-C₃)haloalkyl or hydroxy(C₁-C₃)alkyl;

X^{C4} represents H, (C₁-C₃)alkyl, halogen or hydroxy;

X^{D1} represents H, (C₁-C₄)alkyl, ω-(C₂-C₃)haloalkyl or ω-hydroxy(C₂-C₄)alkyl;

X^{D2} and X^{D3} each independently represent H, (C₁-C₄)alkyl, (C₁-C₃)haloalkyl or hydroxy(C₁-C₃)alkyl;

X^{E1} represents H, (C₁-C₄)alkyl, (C₁-C₃)haloalkyl, 1,2-dihydroxyethyl or hydroxy(C₁-C₃)alkyl;

X^{F1} represents H, (C₁-C₄)alkyl, (C₁-C₃)haloalkyl, 1,2-dihydroxyethyl or hydroxy(C₁-C₃)alkyl; and

one of V or W represents —O—, —CH(OH)— or —CH₂—, and the other represents —CH₂—;

R¹ represents H, PO₃H₂, SO₃H, phosphonooxymethyl or the group L represented below

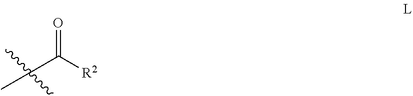

wherein R² represents (C₁-C₄)alkylamino(C₁-C₄)alkyl, [di(C₁-C₄)alkylamino](C₁-C₄)alkyl, phosphonooxy(C₁-C₄)alkyl, phosphonooxymethoxy, 2-(phosphonooxy-(C₁-C₄)alkyl)-phenyl, (2-(phosphonooxy)-phenyl)-(C₁-C₄)alkyl (especially 2-(2-(phosphonooxy)-phenyl)-ethyl) or [2-(phosphonooxy-(C₁-C₄)alkyl)-phenyl]-(C₁-C₄)alkyl;

and to salts (in particular pharmaceutically acceptable salts) of such compounds of formula I.

It is understood that groups —O—R¹ in the fragment —CO—NH—O—R¹ wherein R¹ is not H, or derivatives of a hydroxy group when present in a group M, such as phosphonooxy, (di(C₁-C₄)alkylamino)-(C₁-C₃)alkyl-carbonyloxy (e.g. dimethylaminoacetoxy), [(2-(phosphonooxy-(C₁-C₄)alkyl)-phenyl)-(C₁-C₄)alkyl]-carbonyloxy, [2-(phosphonooxy-(C₁-C₄)alkyl)-phenyl]-carbonyloxy, or [(2-phosphonooxy-phenyl)-(C₁-C₄)alkyl]-carbonyloxy (e.g. [2-(2-phosphonooxy-phenyl)-ethyl]-carbonyloxy) represent prodrugs of the corresponding —CO—NH—OH group, respectively, the corresponding hydroxy group. The term prodrug in the context of a —CO—NH—OH or hydroxy group, thus preferably refers to the above-mentioned groups.

In particular:
the prodrug group (di(C₁-C₄)alkylamino)-(C₁-C₃)alkyl-carbonyloxy (occurring when R² represents [di(C₁-C₄)alkylamino](C₁-C₄)alkyl)) notably refers to dimethylaminoacetoxy;
the prodrug group [2-(phosphonooxy-(C₁-C₄)alkyl)-phenyl]-carbonyloxy (occurring when R² represents 2-(phosphonooxy-(C₁-C₄)alkyl)-phenyl) notably refers to one of the groups represented below

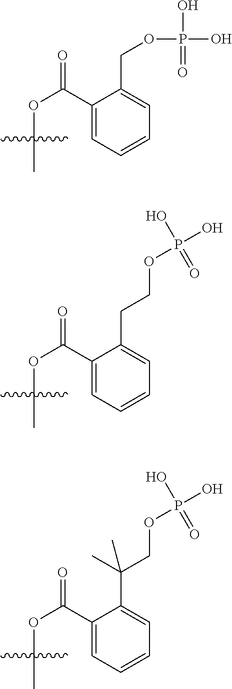 [SMILES: *OC(=O)c1ccccc1COP(=O)(O)O ; *OC(=O)c1ccccc1CCOP(=O)(O)O ; *OC(=O)c1ccccc1C(C)(C)COP(=O)(O)O]

the prodrug group [(2-phosphonooxy-phenyl)-(C₁-C₄)alkyl]-carbonyloxy (occurring when R² represents (2-(phosphonooxy)-phenyl)-(C₁-C₄)alkyl) notably refers to one of the groups represented below

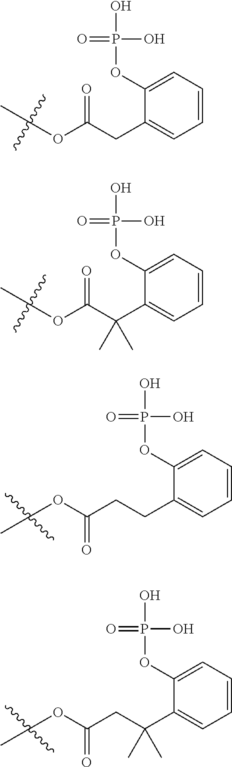

The following paragraphs provide definitions of the various chemical moieties for the compounds according to the invention and are intended to apply uniformly throughout the specification and claims, unless an otherwise expressly set out definition provides a broader or narrower definition:
The term “halogen” refers to fluorine, chlorine, bromine or iodine, and preferably to fluorine or chlorine, and most preferably to fluorine.
The term “alkyl”, used alone or in combination, refers to a straight or branched chain alkyl group containing from one to four carbon atoms. The term “(Cₓ-C_{y})alkyl” (x and y each being an integer) refers to a straight or branched chain alkyl group containing x to y carbon atoms. For example, a (C₁-C₄)alkyl group contains from one to three carbon atoms. Examples of (C₁-C₄)alkyl groups include, but are not limited to, methyl, ethyl, n-propyl, iso-propyl, n-butyl and tert-butyl.
The term “haloalkyl”, used alone or in combination, refers to an alkyl group as defined before wherein one or more hydrogen atoms (and possibly all) has or have been replaced by halogen atoms as defined before. The term “(Cₓ-C_{y})haloalkyl” (x and y each being an integer) refers to a haloalkyl group containing x to y carbon atoms. For example, a (C₁-C₃)haloalkyl group contains from one to three carbon atoms. Examples of (C₁-C₃)haloalkyl groups are fluoromethyl, difluoromethyl, trifluoromethyl, 2-fluoro-ethyl, 2-chloro-ethyl, 2-bromo-ethyl, 3-fluoro-propyl, 3-chloro-propyl, 3-bromo-propyl, 4-fluoro-butyl, 4-chloro-butyl and 4-bromo-butyl.
The term “ω-halo(Cₓ-C_{y})alkyl” (x and y each being an integer), used alone or in combination, refers herein to a haloalkyl group as defined before which contains x to y carbon atoms and wherein only one hydrogen atom has been replaced by a halogen atom and the hydrogen atom that has been replaced by a halogen group always originates from one methyl part of the alkyl group (that is, the ω-halo(Cₓ-C_{y})alkyl group is always such that it contains a —CH₂X group wherein X is halogen). For example, a ω-halo(C₂-C₄)alkyl group is a haloalkyl group as defined before which contains from two to four carbon atoms and wherein only one hydrogen atom has been replaced by a halogen atom and the hydrogen atom that has been replaced by a halogen group always originates from one methyl part of the (C₂-C₄)alkyl group. Examples of ω-halo(C₂-C₄)alkyl groups include, but are not limited to, 2-fluoro-ethyl, 2-chloro-ethyl, 2-bromo-ethyl, 3-fluoro-propyl, 3-chloro-propyl, 3-bromo-propyl, 4-fluoro-butyl, 4-chloro-butyl and 4-bromo-butyl.
The term “hydroxyalkyl”, used alone or in combination, refers to an alkyl group as defined before wherein one hydrogen atom has been replaced by a hydroxy group. The term “hydroxy(Cₓ-C_{y})alkyl” (x and y each being an integer) refers to a hydroxyalkyl group as defined which contains x to y carbon atoms. For example, a hydroxy(C₁-C₃)alkyl group is a hydroxyalkyl group as defined before which contains from one to three carbon atoms.

Examples of hydroxy(C₁-C₃)alkyl groups include, but are not limited to, hydroxymethyl, 2-hydroxy-ethyl, 2-hydroxy-propyl and 3-hydroxy-propyl.
The term “ω-hydroxy(Cₓ-C_{y})alkyl” (x and y each being an integer), used alone or in combination, refers herein to a hydroxyalkyl group as defined before which contains x to y carbon atoms and wherein the hydrogen atom that has been replaced by a hydroxy group always originates from one methyl part of the alkyl group (that is, the ω-hydroxy(Cₓ-C_{y})alkyl group is always such that it contains a —CH₂OH group). For example, a ω-hydroxy(C₂-C₄)alkyl group is a hydroxyalkyl group as defined before which contains from two to four carbon atoms and wherein the hydrogen atom that has been replaced by a hydroxy group always originates from one methyl part of the (C₂-C₄)alkyl group. Examples of ω-hydroxy(C₂-C₄)alkyl groups include, but are not limited to, 2-hydroxy-ethyl, 3-hydroxy-propyl and 4-hydroxy-butyl.
The term “phosphonooxyalkyl”, used alone or in combination, refers to an alkyl group as defined before wherein one hydrogen atom has been replaced by a phosphonooxy group. The term “phosphonooxy(Cₓ-C_{y})alkyl” (x and y each being an integer) refers to a phosphonooxyalkyl group as defined which contains x to y carbon atoms. For example, a phosphonooxy(C₂-C₄)alkyl group is a phosphonooxyalkyl group as defined before which contains from two to four carbon atoms.
The term “ω-phosphonooxy(Cₓ-C_{y})alkyl” (x and y each being an integer) refers herein to an phosphonooxy group as defined before which contains x to y carbon atoms and wherein the hydrogen atom that has been replaced by a phosphonooxy group always originates from one methyl part of the alkyl group (that is, the ω-phosphonooxy(Cₓ-C_{y})alkyl group is always such that it contains a —CH₂—OP(O)(OH)₂ group). For example, a ω-phosphonooxy(C₂-C₄)alkyl group is a phosphonooxyalkyl group as defined before which contains from two to four carbon atoms and wherein the hydrogen atom that has been replaced by a phosphonooxy group always originates from one methyl part of the (C₂-C₄)alkyl group. Examples of ω-hydroxy(C₂-C₄)alkyl groups include, but are not limited to, 2-phosphonooxy-ethyl, 3-phosphonooxy-propyl and 4-phosphonooxy-butyl.
The term “cycloalkyl”, used alone or in combination, refers to a saturated cyclic hydrocarbon moiety containing 3 to 6 carbon atoms. The term “(Cₓ-C_{y})cycloalkyl” (x and y each being an integer) refers to a cycloalkyl group as defined before containing x to y carbon atoms. For example, a (C₃-C₆)cycloalkyl group contains from three to six carbon atoms. Representative examples of (C₃-C₆)cycloalkyl groups include, but are not limited to, cyclopropyl and cyclopentyl.
The term “quinolone-resistant”, when used in this text, refers to a bacterial strain against which ciprofloxacin has a Minimal Inhibitory Concentration of at least 16 mg/L (said Minimal Inhibitory Concentration being measured with the standard method described in “Methods for Dilution Antimicrobial Susceptibility Tests for Bacteria that Grow Aerobically”, Approved standard, 7^{th} ed., Clinical and Laboratory Standards Institute (CLSI) Document M7-A7, Wayne, Pa., USA (2006)).
The term “carbapenem-resistant”, when used in this text, refers to a bacterial strain against which imipenem has a Minimal Inhibitory Concentration of at least 16 mg/L (said Minimal Inhibitory Concentration being measured with the standard method described in “Methods for Dilution Antimicrobial Susceptibility Tests for Bacteria that Grow Aerobically”, Approved standard, 7th ed., Clinical and Laboratory Standards Institute (CLSI) Document M7-A7, Wayne, Pa., USA (2006)).
The term “multi-drug resistant”, when used in this text, refers to a bacterial strain against which at least three antibiotic compounds selected from three distinct antibiotic categories have Minimal Inhibitory Concentrations (MICs) over their respective clinical breakpoints, whereby said three distinct antibiotic categories are chosen among penicillins, combinations of penicillins with beta-lactamase inhibitors, cephalosporins, carbapenems, monobactams, fluoro-quinolones, aminoglycosides, phosphonic acids, tetracyclins and polymixins. Clinical breakpoints are defined according to the latest available list published by Clinical and Laboratory Standards Institute (Wayne, Pa., USA). Accordingly, clinical breakpoints are the levels of MIC at which, at a given time, a bacterium is deemed either susceptible or resistant to treatment by the corresponding antibiotic or antibiotic combination.

Any reference hereinbefore or hereinafter to a compound of formula I is to be understood as referring also to salts, especially pharmaceutically acceptable salts, of a compound of formula I, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example ‘Handbook of Pharmaceutical Salts. Properties, Selection and Use.’, P. Heinrich Stahl, Camille G. Wermuth (Eds.), Wiley-VCH (2008) and ‘Pharmaceutical Salts and Co-crystals’, Johan Wouters and Luc Quere (Eds.), RSC Publishing (2012).

In this text, a bond interrupted by a wavy line shows a point of attachment of the radical drawn to the rest of the molecule. For example, the radical drawn below

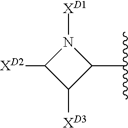

wherein each of X^{D1}, X^{D2} and X^{D3} represents H is the 2-azetidin-1-yl group.

Besides, the term “room temperature” as used herein refers to a temperature of 25° C.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., and preferably to an interval extending from Y minus 5° C. to Y plus 5° C.

2) The invention in particular relates to compounds of formula I according to embodiment 1) which are also compounds of formula I_{CE}

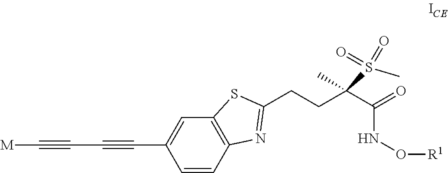

wherein

M is the group (4-hydroxypiperidin-1-yl)carbonyloxymethyl, (3-hydroxyazetidin-1-yl)carbonyloxymethyl or 1-(methylamino)cyclopropyl, or M represents one of the groups M^{A}, M^{B}, M^{C}, M^{D} and M^{E} represented below

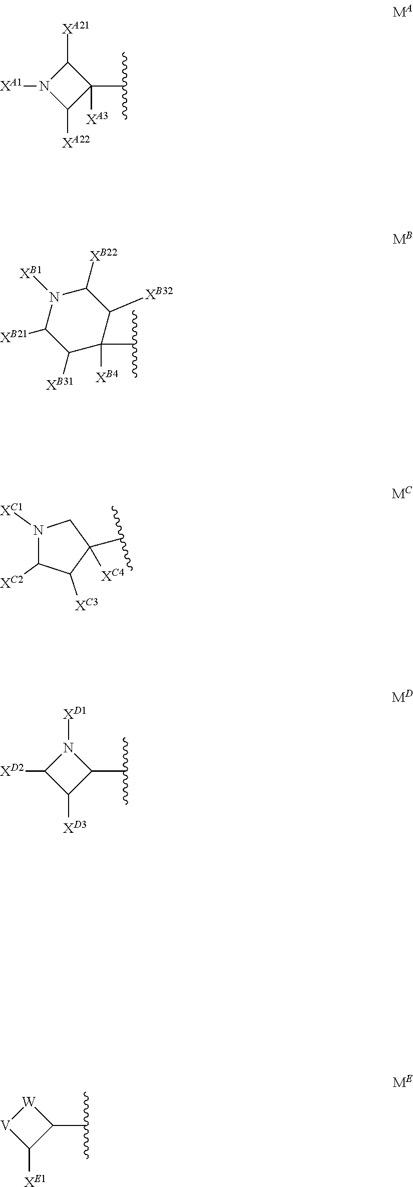

wherein

X^{A1} represents ((C₁-C₄)alkyl, ω-(C₂-C₃)haloalkyl, ω-hydroxy(C₂-C₄)alkyl, oxetan-3-yl, (oxetan-3-yl)methyl, (C₃-C₆)cycloalkyl, 3-hydroxycyclobut-1-yl, or ω-phosphonooxy-(C₂-C₄)alkyl;

one of X^{A21} and X^{A22} represents H and the other represents H, (C₁-C₄)alkyl or hydroxy(C₁-C₃)alkyl;

X^{A3} represents H;

X^{B1} represents (C₁-C₄)alkyl;

each of X^{B21} and X^{B22} represents H;

each of X^{B31} and X^{B32} represents H;

X^{B4} represents halogen;

X^{C1} represents (C₁-C₄)alkyl;

X^{C2} represents H or hydroxy(C₁-C₃)alkyl;

X^{C3} represents H;

X^{C4} represents H or halogen;

X^{D1} represents (C₁-C₄)alkyl;

X^{D2} represents H and X^{D3} represents H or hydroxy(C₁-C₃)alkyl;

X^{E1} represents hydroxy(C₁-C₃)alkyl; and

each of V and W represents —CH₂—;

R¹ represents H or the group L represented below

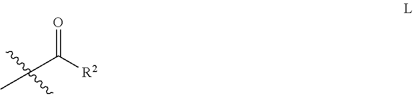

wherein R² represents (2-(phosphonooxy)-phenyl)-(C₁-C₄)alkyl (especially 2-(2-(phosphonooxy)-phenyl)-ethyl);

and to salts (in particular pharmaceutically acceptable salts) of compounds of formula I_{CE}.

3) One particular sub-embodiment of embodiment 1) or 2) relates to the compounds of formula I as defined in embodiment 1) or 2) wherein R¹ represents H.

4) Another sub-embodiment of embodiment 1) or 2) relates to the compounds of formula I as defined in embodiment 1) or 2) wherein R¹ does not represent H.

5) According to one main embodiment of this invention, the compounds of formula I as defined in embodiments 1) to 4) will be such that M is the group (4-hydroxypiperidin-1-yl)carbonyloxymethyl, (3-hydroxyazetidin-1-yl)carbonyloxymethyl or 1-(methylamino)cyclopropyl.

6) Preferably, the compounds of formula I as defined in embodiment 5) will be such that M is the group 1-(methylamino)cyclopropyl.

7) According to another embodiment of this invention, the compounds of formula I as defined in embodiments 1) to 4) will be such that M is the group M^{A}.

8) Preferably, the compounds of formula I as defined in embodiment 7) will be such that:
X^{A1} represents (C₁-C₄)alkyl, ω-(C₂-C₃)haloalkyl, ω-hydroxy(C₂-C₄)alkyl, oxetan-3-yl, (oxetan-3-yl)methyl, (C₃-C₆)cycloalkyl, 3-hydroxycyclobut-1-yl, or ω-phosphonooxy-(C₂-C₄)alkyl;
one of X^{A21} and X^{A22} represents H and the other represents H or (C₁-C₄)alkyl or hydroxy(C₁-C₃)alkyl; and
X^{A3} represents H.

9) More preferably, the compounds of formula I as defined in embodiment 7) will be such that:
X^{A1} represents ω-hydroxy(C₂-C₄)alkyl, oxetan-3-yl, (oxetan-3-yl)methyl, (C₃-C₆)cycloalkyl or 3-hydroxycyclobut-1-yl;
one of X^{A21} and X^{A22} represents H and the other represents H or methyl; and
X^{A3} represents H.

10) Even more preferably, the compounds of formula I as defined in embodiment 7) will be such that:
X^{A1} represents ω-hydroxy(C₂-C₄)alkyl, oxetan-3-yl, (oxetan-3-yl)methyl or 3-hydroxycyclobut-1-yl;
one of X^{A21} and X^{A22} represents H and the other represents H or methyl; and
X^{A3} represents H.

11) According to another main embodiment of this invention, the compounds of formula I as defined in embodiments 1) to 4) will be such that M is the group M^{B}.

12) Preferably, the compounds of formula I as defined in embodiment 11) will be such that:
X^{B1} represents (C₁-C₄)alkyl;
each of X^{B21} and X^{B22} represents H;
each of X^{B31} and X^{B32} represents H; and
X^{B4} represents halogen.

13) More preferably, the compounds of formula I as defined in embodiment 11) will be such that:
X^{B1} represents methyl;
each of X^{B21} and X^{B22} represents H;
each of X^{B31} and X^{B32} represents H; and
X^{B4} represents halogen.

14) Even more preferably, the compounds of formula I as defined in embodiment 11) will be such that:
X^{B1} represents methyl;
each of X^{B21} and X^{B22} represents H;
each of X^{B31} and X^{B32} represents H; and
X^{B4} represents fluorine.

15) According to yet another main embodiment of this invention, the compounds of formula I as defined in embodiments 1) to 4) will be such that M is the group M^{C}.

16) Preferably, the compounds of formula I as defined in embodiment 15) will be such that:
X^{C1} represents (C₁-C₄)alkyl;
X^{C2} represents H or hydroxy(C₁-C₃)alkyl;
X^{C3} represents H; and
X^{C4} represents H or halogen.

17) More preferably, the compounds of formula I as defined in embodiment 15) will be such that:
X^{C1} represents methyl;
X^{C2} represents H;
X^{C3} represents H; and
X^{C4} represents halogen.

18) Even more preferably, the compounds of formula I as defined in embodiment 15) will be such that:
X^{C1} represents methyl;
X^{C2} represents H;
X^{C3} represents H; and
X^{C4} represents fluorine.

19) According to yet another main embodiment of this invention, the compounds of formula I as defined in embodiments 1) to 4) will be such that M is the group M^{D}.

20) Preferably, the compounds of formula I as defined in embodiment 19) will be such that:
X^{D1} represents (C₁-C₄)alkyl;
X^{D2} represents H; and
X^{D3} represents H or hydroxy(C₁-C₃)alkyl.

21) More preferably, the compounds of formula I as defined in embodiment 19) will be such that:
X^{D1} represents methyl;
X^{D2} represents H; and
X^{D3} represents hydroxy(C₁-C₃)alkyl.

22) Even more preferably, the compounds of formula I as defined in embodiment 19) will be such that:
X^{D1} represents methyl;
X^{D2} represents H; and
X^{D3} represents hydroxymethyl or 2-hydroxy-ethyl (especially hydroxymethyl).

23) According to yet another main embodiment of this invention, the compounds of formula I as defined in embodiments 1) to 4) will be such that M is the group M^{E}.

24) Preferably, the compounds of formula I as defined in embodiment 23) will be such that:
X^{E1} represents hydroxy(C₁-C₃)alkyl; and
each of V and W represents —CH₂—.

25) More preferably, the compounds of formula I as defined in embodiment 23) will be such that:
X^{E1} represents hydroxymethyl or 2-hydroxy-ethyl (especially hydroxymethyl); and
each of V and W represents —CH₂—.

26) According to yet another main embodiment of this invention, the compounds of formula I as defined in embodiment 1), 3) or 4) will be such that M is the group M^{F}.

27) In particular, the compounds of formula I as defined in embodiment 26) will be such that each of V and W represents —CH₂—.

28) One further embodiment of this invention relates to the compounds of formula I as defined in embodiment 1) or 2) wherein:
M is the group (4-hydroxypiperidin-1-yl)carbonyloxymethyl, (3-hydroxyazetidin-1-yl)carbonyloxymethyl or 1-(methylamino)cyclopropyl, or M represents one of the groups M^{A}, M^{B}, M^{C}, M^{D} and M^{E} wherein
   X^{A1} represents ((C₁-C₄)alkyl, ω-(C₂-C₃)haloalkyl, ω-hydroxy(C₂-C₄)alkyl, oxetan-3-yl, (oxetan-3-yl)methyl, (C₃-C₆)cycloalkyl, 3-hydroxycyclobut-1-yl; one of X^{A21} and X^{A22} represents H and the other represents H, (C₁-C₄)alkyl or hydroxy(C₁-C₃)alkyl; and X^{A3} represents H;
   X^{B1} represents (C₁-C₄)alkyl; each of X^{B21} and X^{B22} represents H; each of X^{B3}1 and X^{B32} represents H; and X^{B4} represents halogen;
   X^{C1} represents (C₁-C₄)alkyl; X^{C2} represents H or hydroxy(C₁-C₃)alkyl; X^{C3} represents H; and X^{C4} represents H or halogen;
   X^{D1} represents (C₁-C₄)alkyl; X^{D2} represents H; and X^{D3} represents H or hydroxy(C₁-C₃)alkyl;
   X^{E1} represents hydroxy(C₁-C₃)alkyl; and each of V and W represents —CH₂—; and
R¹ represents H.

29) Preferably, the compounds of formula I according to embodiment 28) will be such that:
M is the group (4-hydroxypiperidin-1-yl)carbonyloxymethyl, (3-hydroxyazetidin-1-yl)carbonyloxymethyl or 1-(methylamino)cyclopropyl; or
M represents the group M^{A} wherein X^{A1} represents ω-hydroxy(C₂-C₄)alkyl, oxetan-3-yl, (oxetan-3-yl)methyl, (C₃-C₆)cycloalkyl, 3-hydroxycyclobut-1-yl; one of X^{A21} and X^{A22} represents H and the other represents H or (C₁-C₄)alkyl; and X^{A3} represents H; or
M represents the group M^{C} wherein X^{C1} represents (C₁-C₄)alkyl; X^{C2} represents H; X^{C3} represents H; and X^{C4} represents halogen;
M represents the group M^{D} wherein X^{D1} represents (C₁-C₄)alkyl; X^{D2} represents H; and X^{D3} represents hydroxy(C₁-C₃)alkyl;
M represents the group M^{E} wherein X^{E1} represents hydroxy(C₁-C₃)alkyl; and each of V and W represents —CH₂—.

30) More preferably, the compounds of formula I according to embodiment 28) will be such that:
M is the group 1-(methylamino)cyclopropyl; or
M represents the group M^{A} wherein X^{A1} represents ω-hydroxy(C₂-C₄)alkyl, oxetan-3-yl, (oxetan-3-yl)methyl, 3-hydroxycyclobut-1-yl; one of X^{A21} and X^{A22} represents H and the other represents H or (C₁-C₄)alkyl; and X^{A3} represents H; or
M represents the group M^{C} wherein X^{C1} represents (C₁-C₄)alkyl; X^{C2} represents H; X^{C3} represents H; and X^{C4} represents halogen;
M represents the group M^{D} wherein X^{D1} represents (C₁-C₄)alkyl; X^{D2} represents H; and X^{D3} represents hydroxy(C₁-C₃)alkyl;
M represents the group M^{E} wherein X^{E1} represents hydroxy(C₁-C₃)alkyl; and each of V and W represents —CH₂—.

31) Yet one further embodiment of this invention relates to the compounds of formula I as defined in embodiment 1) or 2) wherein:
M represents the group M^{A} wherein X^{A1} represents ω-phosphonooxy-(C₂-C₄)alkyl; one of X^{A21} and X^{A22} represents H and the other represents H, (C₁-C₄)alkyl or hydroxy(C₁-C₃)alkyl; and X^{A3} represents H; and
R¹ represents H;
OR
M is the group 4-hydroxypiperidin-1-yl)carbonyloxymethyl, (3-hydroxyazetidin-1-yl)carbonyloxymethyl or 1-(methylamino)cyclopropyl, or M represents one of the groups M^{A}, M^{B}, M^{C}, M^{D} and M^{E} wherein
   X^{A1} represents ((C₁-C₄)alkyl, ω-(C₂-C₃)haloalkyl, ω-hydroxy(C₂-C₄)alkyl, oxetan-3-yl, (oxetan-3-yl)methyl, (C₃-C₆)cycloalkyl, 3-hydroxycyclobut-1-yl; one of X^{A21} and X^{A22} represents H and the other represents H, (C₁-C₄)alkyl or hydroxy(C₁-C₃)alkyl; X^{A3} represents H;
   X^{B1} represents (C₁-C₄)alkyl; each of X^{B21} and X^{B22} represents H; each of X^{B31} and X^{B32} represents H; X^{B4} represents halogen;
   X^{C1} represents (C₁-C₄)alkyl; X^{C2} represents H or hydroxy(C₁-C₃)alkyl; X^{C3} represents H; X^{C4} represents H or halogen;
   X^{D1} represents (C₁-C₄)alkyl; X^{D2} represents H and X^{D3} represents H or hydroxy(C₁-C₃)alkyl;
   X^{E1} represents hydroxy(C₁-C₃)alkyl; and
   each of V and W represents —CH₂—; and
R¹ represents the group L wherein R² represents (2-(phosphonooxy)-phenyl)-(C₁-C₄)alkyl (especially 2-(2-(phosphonooxy)-phenyl)-ethyl).

32) Preferably, the compounds of formula I according to embodiment 31) will be such that:
M represents the group M^{A} wherein X^{A1} represents ω-phosphonooxy-(C₂-C₄)alkyl; one of X^{A21} and X^{A22} represents H and the other represents H or methyl; and X^{A3} represents H; and R¹ represents H; or
OR
M is the group M^{A} wherein X^{A1} represents ω-hydroxy(C₂-C₄)alkyl, oxetan-3-yl, (oxetan-3-yl)methyl or 3-hydroxycyclobut-1-yl; one of X^{A21} and X^{A22} represents H and the other represents H or methyl; and X^{A3} represents H; and R¹ represents the group L wherein R² represents (2-(phosphonooxy)-phenyl)-(C₁-C₄)alkyl (especially 2-(2-(phosphonooxy)-phenyl)-ethyl).

33) More preferably, the compounds of formula I according to embodiment 31) will be such that:
M represents the group M^{A} wherein X^{A1} represents ω-phosphonooxy-(C₂-C₄)alkyl, each of X^{A21} and X^{A22} represents H and X^{A3} represents H; and R¹ represents H; or
OR
M is the group M^{A} wherein X^{A1} represents co-hydroxy(C₂-C₄)alkyl, each of X^{A21} and X^{A22} represents H and X^{A3} represents H; and R¹ represents the group L wherein R² represents 2-(2-(phosphonooxy)-phenyl)-ethyl.

34) Even more preferably, the compounds of formula I according to embodiment 31) will be such that:
M represents the group M^{A} wherein X^{A1} represents 2-phosphonooxy-ethyl, each of X^{A21} and X^{A22} represents H and X^{A3} represents H; and R¹ represents H; or
OR
M is the group M^{A} wherein X^{A1} represents 3-hydroxy-propyl, each of X^{A21} and X^{A22} represents H and X^{A3} represents H; and R¹ represents the group L wherein R² represents 2-(2-(phosphonooxy)-phenyl)-ethyl.

35) Another embodiment of this invention relates to compounds of formula I as defined in one of embodiments 1) to 34) as well as to isotopically labelled, especially ²H (deuterium) labelled compounds of formula I as defined in one of embodiments 1) to 34), which compounds are identical to the compounds of formula I as defined in one of embodiments 1) to 34) except that, when X^{A1} does not represent methyl-d or methyl-d2, one or more atoms has or have each been replaced by an atom having the same atomic number but an atomic mass different from the atomic mass usually found in nature. Isotopically labelled, especially ²H (deuterium) labelled compounds of formula I and salts (in particular pharmaceutically acceptable salts) thereof are thus within the scope of the present invention. Substitution of hydrogen with the heavier isotope ²H (deuterium) may lead to greater metabolic stability, resulting e.g. in an increased in-vivo half-life, reduced dosage requirements, or an improved safety profile. In one variant of the invention, the compounds of formula I are not isotopically labelled, or they are labelled only with one or more deuterium atoms. Isotopically labelled compounds of formula I may be prepared in analogy to the methods described hereinafter, but using the appropriate isotopic variation of suitable reagents or starting materials.

36) Another embodiment of the invention relates to a compound of formula I according to embodiment 1) or 2) selected from the group consisting of:
(2R)—N-hydroxy-2-methyl-2-(methylsulfonyl)-4-(6-((1-(oxetan-3-yl)azetidin-3-yl)buta-1,3-diyn-1-yl)benzo[d]thiazol-2-yl)butanamide;
(2R)-5-(2-(4-(hydroxyamino)-3-methyl-3-(methylsulfonyl)-4-oxobutyl)benzo[d]thiazol-6-yl)penta-2,4-diyn-1-yl 4-hydroxypiperidine-1-carboxylate;
(2R)-5-(2-(4-(hydroxyamino)-3-methyl-3-(methylsulfonyl)-4-oxobutyl)benzo[d]thiazol-6-yl)penta-2,4-diyn-1-yl 3-hydroxyazetidine-1-carboxylate;
(2R)—N-hydroxy-2-methyl-4-(6-((1-methylazetidin-3-yl)buta-1,3-diyn-1-yl)benzo[d]thiazol-2-yl)-2-(methyl sulfonyl)butanamide;
(2R)—N-hydroxy-4-(6-((1-(2-hydroxyethyl)azetidin-3-yl)buta-1,3-diyn-1-yl)benzo[d]thiazol-2-yl)-2-methyl-2-(methyl sulfonyl)butanamide;
(R)-2-(3-((2-(4-(hydroxyamino)-3-methyl-3-(methylsulfonyl)-4-oxobutyl)benzo[d]thiazol-6-yl)buta-1,3-diyn-1-yl)azetidin-1-yl)ethyl dihydrogen phosphate;
(2R)—N-hydroxy-4-(6-(((1R,2R)-2-(hydroxymethyl)cyclobutyl)buta-1,3-diyn-1-yl)benzo[d]thiazol-2-yl)-2-methyl-2-(methyl sulfonyl)butanamide;
(2R)—N-hydroxy-2-methyl-4-(6-(((2S)-1-methylazetidin-2-yl)buta-1,3-diyn-1-yl)benzo[d]thiazol-2-yl)-2-(methyl sulfonyl)butanamide;
(2R)—N-hydroxy-2-methyl-2-(methylsulfonyl)-4-(6-((1-(oxetan-3-ylmethyl)azetidin-3-yl)buta-1,3-diyn-1-yl)benzo[d]thiazol-2-yl)butanamide;
(2R)—N-hydroxy-4-(6-((1-(3-hydroxypropyl)azetidin-3-yl)buta-1,3-diyn-1-yl)benzo[d]thiazol-2-yl)-2-methyl-2-(methyl sulfonyl)butanamide;
(2R)-4-(6-((1-cyclopropylazetidin-3-yl)buta-1,3-diyn-1-yl)benzo[d]thiazol-2-yl)-N-hydroxy-2-methyl-2-(methyl sulfonyl)butanamide;
(2R)—N-hydroxy-4-(6-((1-(3-hydroxycyclobutyl)azetidin-3-yl)buta-1,3-diyn-1-yl)benzo[d]thiazol-2-yl)-2-methyl-2-(methyl sulfonyl)butanamide;
(2R)-4-(6-((1-(2-fluoroethyl)azetidin-3-yl)buta-1,3-diyn-1-yl)benzo[d]thiazol-2-yl)-N-hydroxy-2-methyl-2-(methyl sulfonyl)butanamide;
(2R)-4-(6-(((3-fluoro-1-methylpyrrolidin-3-yl)buta-1,3-diyn-1-yl)benzo[d]thiazol-2-yl)-N-hydroxy-2-methyl-2-(methyl sulfonyl)butanamide;
(2R)-4-(6-((4-fluoro-1-methylpiperidin-4-yl)buta-1,3-diyn-1-yl)benzo[d]thiazol-2-yl)-N-hydroxy-2-methyl-2-(methyl sulfonyl)butanamide;
(2R)-4-(6-(((2R,3S)-1,2-dimethylazetidin-3-yl)buta-1,3-diyn-1-yl)benzo[d]thiazol-2-yl)-N-hydroxy-2-methyl-2-(methyl sulfonyl)butanamide;
(2R)—N-hydroxy-4-(6-(((2R,3R)-2-(hydroxymethyl)-1-methylazetidin-3-yl)buta-1,3-diyn-1-yl)benzo[d]thiazol-2-yl)-2-methyl-2-(methyl sulfonyl)butanamide;
(2R)—N-hydroxy-2-methyl-4-(6-((1-(methylamino)cyclopropyl)buta-1,3-diyn-1-yl)benzo[d]thiazol-2-yl)-2-(methyl sulfonyl)butanamide;
(2R)—N-hydroxy-4-(6-(((2R,3R)-2-(hydroxymethyl)-1-methylazetidin-3-yl)buta-1,3-diyn-1-yl)benzo[d]thiazol-2-yl)-2-methyl-2-(methyl sulfonyl)butanamide;
(2R)—N-hydroxy-4-(6-(((3R,5R)-5-(hydroxymethyl)-1-methylpyrrolidin-3-yl)buta-1,3-diyn-1-yl)benzo[d]thiazol-2-yl)-2-methyl-2-(methyl sulfonyl)butanamide; and
(2R)-2-(3-((4-(5-((1-(3-hydroxypropyl)azetidin-3-yl)buta-1,3-diyn-1-yl)benzo[d]thiazol-2-yl)-2-methyl-2-(methylsulfonyl)butanamido)oxy)-3-oxopropyl)phenyl dihydrogen phosphate;

and to salts (in particular the pharmaceutically acceptable salts) of such compounds.

37) The invention further relates to the groups of compounds of formula I selected from the group consisting of the compounds listed in embodiment 36), which groups of compounds furthermore correspond to one of embodiments 1) to 34), as well as to the salts (in particular the pharmaceutically acceptable salts) of such compounds. The invention moreover relates to any individual compound of formula I selected from the group consisting of the compounds listed in embodiment 36), and to the salts (in particular the pharmaceutically acceptable salts) of such individual compound.

The compounds of formula I according to this invention, i.e. according to one of embodiments 1) to 37) above, exhibit antibacterial activity, especially against Gram-negative organisms and are therefore suitable to treat bacterial infections in mammals, especially humans. Said compounds may also be used for veterinary applications, such as treating infections in livestock and companion animals. They may further constitute substances for preserving inorganic and organic materials in particular all types of organic materials for example polymers, lubricants, paints, fibres, leather, paper and wood.

They may therefore be used for the treatment or prevention of infectious disorders caused by fermentative or non-fermentative gram negative bacteria, especially those caused by susceptible and multi-drug resistant Gram-negative bacteria. Examples of such Gram-negative bacteria include Acinetobacter spp. such as Acinetobacter baumannii or Acinetobacter haemolyticus, Actinobacillus actinomycetemcomitans, Achromobacter spp. such as Achromobacter xylosoxidans or Achromobacter faecalis, Aeromonas spp. such as Aeromonas hydrophila, Bacteroides spp. such as Bacteroides fragilis, Bacteroides theataioatamicron, Bacteroides distasonis, Bacteroides ovatus or Bacteroides vulgatus, Bartonella hensenae, Bordetella spp. such as Bordetella pertussis, Borrelia spp. such as Borrelia Burgdorferi, Brucella spp. such as Brucella melitensis, Burkholderia spp. such as Burkholderia cepacia, Burkholderia pseudomallei or Burkholderia mallei, Campylobacter spp. such as Campylobacter jejuni, Campylobacter fetus or Campylobacter coli, Cedecea, Chlamydia spp. such as Chlamydia pneumoniae, Chlamydia trachomatis, Citrobacter spp. such as Citrobacter diversus (koseri) or Citrobacter freundii, Coxiella burnetii, Edwardsiella spp. such as Edwarsiella tarda, Ehrlichia chafeensis, Eikenella corrodens, Enterobacter spp. such as Enterobacter cloacae, Enterobacter aerogenes, Enterobacter agglomerans, Escherichia coli, Francisella tularensis, Fusobacterium spp., Haemophilus spp. such as Haemophilus influenzae (beta-lactamase positive and negative) or Haemophilus ducreyi, Helicobacter pylori, Kingella kingae, Klebsiella spp. such as Klebsiella oxytoca, Klebsiella pneumoniae (including those encoding extended-spectrum beta-lactamases (hereinafter “ESBLs”), carbapenemases (KPCs), cefotaximase-Munich (CTX-M), metallo-beta-lactamases, and AmpC-type beta-lactamases that confer resistance to currently available cephalosporins, cephamycins, carbapenems, beta-lactams, and beta-lactam/beta-lactamase inhibitor combinations), Klebsiella rhinoscleromatis or Klebsiella ozaenae, Legionella pneumophila, Mannheimia haemolyticus, Moraxella catarrhalis (beta-lactamase positive and negative), Morganella morganii, Neisseria spp. such as Neisseria gonorrhoeae or Neisseria meningitidis, Pasteurella spp. such as Pasteurella multocida, Plesiomonas shigelloides, Porphyromonas spp. such as Porphyromonas asaccharolytica, Prevotella spp. such as Prevotella corporis, Prevotella intermedia or Prevotella endodontalis, Proteus spp. such as Proteus mirabilis, Proteus vulgaris, Proteus penneri or Proteus myxofaciens, Porphyromonas asaccharolytica, Plesiomonas shigelloides, Providencia spp. such as Providencia stuartii, Providencia rettgeri or Providencia alcalifaciens, Pseudomonas spp. such as Pseudomonas aeruginosa (including ceftazidime-, cefpirome- and cefepime-resistant P. aeruginosa, carbapenem-resistant P. aeruginosa or quinolone-resistant P. aeruginosa) or Pseudomonas fluorescens, Ricketsia prowazekii, Salmonella spp. such as Salmonella typhi or Salmonella paratyphi, Serratia marcescens, Shigella spp. such as Shigella flexneri, Shigella boydii, Shigella sonnei or Shigella dysenteriae, Streptobacillus moniliformis, Stenotrophomonas maltophilia, Treponema spp., Vibrio spp. such as Vibrio cholerae, Vibrio parahaemolyticus, Vibrio vulnificus, Vibrio alginolyticus, Yersinia spp. such as Yersinia enterocolitica, Yersinia pestis or Yersinia pseudotuberculosis.

The compounds of formula I according to this invention are thus useful for treating a variety of infections caused by fermentative or non-fermentative Gram-negative bacteria, especially infections such as: nosocomial pneumonia (related to infection by Legionella pneumophila, Haemophilus influenzae, or Chlamydia pneumonia); urinary tract infections; systemic infections (bacteraemia and sepsis); skin and soft tissue infections (including burn patients); surgical infections; intraabdominal infections; lung infections (including those in patients with cystic fibrosis); Helicobacter pylori (and relief of associated gastric complications such as peptic ulcer disease, gastric carcinogenesis, etc.); endocarditis; diabetic foot infections; osteomyelitis; otitis media, sinusitus, bronchitis, tonsillitis, and mastoiditis related to infection by Haemophilus influenzae or Moraxella catarrhalis; pharynigitis, rheumatic fever, and glomerulonephritis related to infection by Actinobacillus haemolyticum; sexually transmitted diseases related to infection by Chlamydia trachormatis, Haemophilus ducreyi, Treponema pallidum, Ureaplasma urealyticum, or Neisseria gonorrheae; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gonorrhoeae or H. influenzae; gastroenteritis related to infection by Campylobacter jejuni; persistent cough related to infection by Bordetella pertussis and gas gangrene related to infection by Bacteroides spp. Other bacterial infections and disorders related to such infections that may be treated or prevented in accord with the method of the present invention are referred to in J. P. Sanford et al., “The Sanford Guide to Antimicrobial Therapy”, 26th Edition, (Antimicrobial Therapy, Inc., 1996).

The preceding lists of infections and pathogens are to be interpreted merely as examples and in no way as limiting.

The compounds of formula I according to this invention, or the pharmaceutically acceptable salts thereof, may therefore be used for the preparation of a medicament, and are suitable, for the prevention or treatment of a bacterial infection, in particular for the prevention or treatment of a bacterial infection caused by Gram-negative bacteria, especially by multi-drug resistant Gram-negative bacteria.

The compounds of formula I according to this invention, or the pharmaceutically acceptable salts thereof, may thus especially be used for the preparation of a medicament, and are suitable, for the prevention or treatment of a bacterial infection caused by Gram-negative bacteria selected from the group consisting of Burkholderia spp. (e.g. Burkholderia cepacia), Citrobacter spp., Enterobacter aerogenes, Enterobacter cloacae, Escherichia coli, Klebsiella oxytoca, Klebsiella pneumoniae, Serratia marcescens, Stenotrophomonas maltophilia and Pseudomonas aeruginosa (notably for the prevention or treatment of a bacterial infection caused by Escherichia coli bacteria, Klebsiella pneumoniae bacteria or Pseudomonas aeruginosa bacteria, and in particular for the prevention or treatment of a bacterial infection mediated by quinolone-resistant, carbapenem-resistant or multi-drug resistant Klebsiella pneumoniae bacteria).

The compounds of formula I according to this invention, or the pharmaceutically acceptable salts thereof, may more especially be used for the preparation of a medicament, and are suitable, for the prevention or treatment of a bacterial infection caused by Gram-negative bacteria selected from the group consisting of Citrobacter spp., Enterobacter aerogenes, Enterobacter cloacae, Escherichia coli, Klebsiella oxytoca, Klebsiella pneumoniae, Serratia marcescens, Stenotrophomonas maltophilia and Pseudomonas aeruginosa bacteria (notably of a bacterial infection caused by Gram-negative bacteria selected from the group consisting of Klebsiella pneumoniae and Pseudomonas aeruginosa bacteria, and in particular of a bacterial infection caused by Pseudomonas aeruginosa bacteria).

The compounds of formula I according to this invention, or the pharmaceutically acceptable salts thereof, may thus especially be used for the preparation of a medicament, and are suitable, for the prevention or treatment of a bacterial infection selected from urinary tract infections, systemic infections (such as bacteraemia and sepsis), skin and soft tissue infections (including burn patients), surgical infections; intraabdominal infections and lung infections (including those in patients with cystic fibrosis).

The compounds of formula I according to this invention, or the pharmaceutically acceptable salts thereof, may more especially be used for the preparation of a medicament, and are suitable, for the prevention or treatment of a bacterial infection selected from urinary tract infections, intraabdominal infections and lung infections (including those in patients with cystic fibrosis), and in particular for the prevention or treatment of a bacterial infection selected from urinary tract infections and intraabdominal infections.

Besides, the compounds of formula I according to this invention display intrinsic antibacterial properties and have the ability to improve permeability of the outer membrane of Gram-negative bacteria to other antibacterial agents. Their use in combination with another antibacterial agent might offer some further advantages such as lowered side-effects of drugs due to lower doses used or shorter time of treatment, more rapid cure of infection shortening hospital stays, increasing spectrum of pathogens controlled, and decreasing incidence of development of resistance to antibiotics. The antibacterial agent for use in combination with a compound of formula I according to this invention will be selected from the group consisting of a penicillin antibiotic (such as ampicillin, piperacillin, penicillin G, amoxicillin, or ticarcillin), a cephalosporin antibiotic (such as ceftriaxone, cefatazidime, cefepime, cefotaxime) a carbapenem antibiotic (such as imipenem, or meropenem), a monobactam antibiotic (such as aztreonam or carumonam), a fluoroquinolone antibiotic (such as ciprofloxacin, moxifloxacin or levofloxacin), a macrolide antibiotic (such as erythromycin or azithromycin), an aminoglycoside antibiotic (such as amikacin, gentamycin or tobramycin), a glycopeptide antibiotic (such as vancomycin or teicoplanin), a tetracycline antibiotic (such as tetracycline, oxytetracycline, doxycycline, minocycline or tigecycline), and linezolid, clindamycin, telavancin, daptomycin, novobiocin, rifampicin and polymyxin. Preferably, the antibacterial agent for use in combination with a compound of formula I according to this invention will be selected from the group consisting of vancomycin, tigecycline and rifampicin.

The compounds of formula I according to this invention, or the pharmaceutically acceptable salt thereof, may moreover be used for the preparation of a medicament, and are suitable, for the prevention or treatment (and especially the treatment) of infections caused by biothreat Gram negative bacterial pathogens as listed by the US Center for Disease Control (the list of such biothreat bacterial pathogens can be found at the web page http://www.selectagents.gov/Select%20Agents%20and%20Toxins%20List.html), and in particular by Gram negative pathogens selected from the group consisting of Yersinia pestis, Francisella tularensis (tularemia), Burkholderia pseudomallei and Burkholderia mallei.

One aspect of this invention therefore relates to the use of a compound of formula I according to one of embodiments 1) to 37), or of a pharmaceutically acceptable salt thereof, for the manufacture of a medicament for the prevention or treatment of a bacterial infection (in particular one of the previously mentioned infections caused by Gram-negative bacteria, especially by multi-drug resistant Gram-negative bacteria). Another aspect of this invention relates to a compound of formula I according to one of embodiments 1) to 37), or a pharmaceutically acceptable salt thereof, for the prevention or treatment of a bacterial infection (in particular for the prevention or treatment of one of the previously mentioned infections caused by Gram-negative bacteria, especially by multi-drug resistant Gram-negative bacteria). Yet another aspect of this invention relates to a compound of formula I according to one of embodiments 1) to 37), or a pharmaceutically acceptable salt thereof, as a medicament. Yet a further aspect of this invention relates to a pharmaceutical composition containing, as active principle, a compound of formula I according to one of embodiments 1) to 37), or a pharmaceutically acceptable salt thereof, and at least one therapeutically inert excipient.

As well as in humans, bacterial infections can also be treated using compounds of formula I (or pharmaceutically acceptable salts thereof) in other species like pigs, ruminants, horses, dogs, cats and poultry.

The present invention also relates to pharmacologically acceptable salts and to compositions and formulations of compounds of formula I.

A pharmaceutical composition according to the present invention contains at least one compound of formula I (or a pharmaceutically acceptable salt thereof) as the active agent and optionally carriers and/or diluents and/or adjuvants, and may also contain additional known antibiotics.

The compounds of formula I and their pharmaceutically acceptable salts can be used as medicaments, e.g. in the form of pharmaceutical compositions for enteral or parenteral administration.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula I or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Another aspect of the invention concerns a method for the prevention or the treatment of a Gram-negative bacterial infection in a patient, comprising the administration to said patient of a pharmaceutically active amount of a compound of formula I according to one of embodiments 1) to 34) or a pharmaceutically acceptable salt thereof. Accordingly, the invention provides a method for the prevention or the treatment of a bacterial infection caused by Gram-negative bacteria (notably for the prevention or treatment of a bacterial infection caused by Escherichia coli bacteria, Klebsiella pneumoniae bacteria or Pseudomonas aeruginosa bacteria, and in particular for the prevention or treatment of a bacterial infection caused by quinolone-resistant, carbapenem-resistant or multi-drug resistant Klebsiella pneumoniae bacteria) in a patient, comprising the administration to said patient of a pharmaceutically active amount of a compound of formula I according to one of embodiments 1) to 37) or a pharmaceutically acceptable salt thereof.

Moreover, the compounds of formula I according to this invention may also be used for cleaning purposes, e.g. to remove pathogenic microbes and bacteria from surgical instruments, catheters and artificial implants or to make a room or an area aseptic. For such purposes, the compounds of formula I could be contained in a solution or in a spray formulation.

This invention, thus, relates to the compounds of formula I as defined in embodiment 1), or further limited under consideration of their respective dependencies by the characteristics of any one of embodiments 2) to 37), and to pharmaceutically acceptable salts thereof. It relates furthermore to the use of such compounds as medicaments, especially for the prevention or treatment of a bacterial infection, in particular for the prevention or treatment of a bacterial infection caused by Gram-negative bacteria (notably for the prevention or treatment of a bacterial infection caused by Escherichia coli bacteria, Klebsiella pneumoniae bacteria or Pseudomonas aeruginosa bacteria, and in particular for the prevention or treatment of a bacterial infection caused by Klebsiella pneumoniae quinolone-resistant, carbapenem-resistant or multi-drug resistant bacteria). The following embodiments relating to compounds of formula I according to embodiment 1) are thus possible and intended and herewith specifically disclosed in individualized form:

1, 2+1, 3+1, 3+2+1, 4+1, 4+2+1, 5+1, 5+2+1, 5+3+1, 5+3+2+1, 5+4+1, 5+4+2+1, 6+5+1, 6+5+2+1, 6+5+3+1, 6+5+3+2+1, 6+5+4+1, 6+5+4+2+1, 7+1, 7+2+1, 7+3+1, 7+3+2+1, 7+4+1, 7+4+2+1, 8+7+1, 8+7+2+1, 8+7+3+1, 8+7+3+2+1, 8+7+4+1, 8+7+4+2+1, 9+7+1, 9+7+2+1, 9+7+3+1, 9+7+3+2+1, 9+7+4+1, 9+7+4+2+1, 10+7+1, 10+7+2+1, 10+7+3+1, 10+7+3+2+1, 10+7+4+1, 10+7+4+2+1, 11+1, 11+2+1, 11+3+1, 11+3+2+1, 11+4+1, 11+4+2+1, 12+11+1, 12+11+2+1, 12+11+3+1, 12+11+3+2+1, 12+11+4+1, 12+11+4+2+1, 13+11+1, 13+11+2+1, 13+11+3+1, 13+11+3+2+1, 13+11+4+1, 13+11+4+2+1, 14+11+1, 14+11+2+1, 14+11+3+1, 14+11+3+2+1, 14+11+4+1, 14+11+4+2+1, 15+1, 15+2+1, 15+3+1, 15+3+2+1, 15+4+1, 15+4+2+1, 16+15+1, 16+15+2+1, 16+15+3+1, 16+15+3+2+1, 16+15+4+1, 16+15+4+2+1, 17+15+1, 17+15+2+1, 17+15+3+1, 17+15+3+2+1, 17+15+4+1, 17+15+4+2+1, 18+15+1, 18+15+2+1, 18+15+3+1, 18+15+3+2+1, 18+15+4+1, 18+15+4+2+1, 19+1, 19+2+1, 19+3+1, 19+3+2+1, 19+4+1, 19+4+2+1, 20+19+1, 20+19+2+1, 20+19+3+1, 20+19+3+2+1, 20+19+4+1, 20+19+4+2+1, 21+19+1, 21+19+2+1, 21+19+3+1, 21+19+3+2+1, 21+19+4+1, 21+19+4+2+1, 22+19+1, 22+19+2+1, 22+19+3+1, 22+19+3+2+1, 22+19+4+1, 22+19+4+2+1, 23+1, 23+2+1, 23+3+1, 23+3+2+1, 23+4+1, 23+4+2+1, 24+23+1, 24+23+2+1, 24+23+3+1, 24+23+3+2+1, 24+23+4+1, 24+23+4+2+1, 25+23+1, 25+23+2+1, 25+23+3+1, 25+23+3+2+1, 25+23+4+1, 25+23+4+2+1, 26+1, 26+3+1, 26+3+2+1, 26+4+1, 26+4+2+1, 27+26+1, 27+26+3+1, 27+26+3+2+1, 27+26+4+1, 27+26+4+2+1, 28+1, 28+2+1, 29+28+1, 29+28+2+1, 30+28+1, 30+28+2+1, 31+1, 31+2+1, 32+31+1, 32+31+2+1, 33+31+1, 33+31+2+1,34+31+1,34+31+2+1,35+1, 35+2+1,35+3+1,35+3+2+1,35+4+1,35+4+2+1,35+5+1,35+5+2+1, 35+5+3+1, 35+5+3+2+1, 35+5+4+1, 35+5+4+2+1, 35+6+5+1, 35+6+5+2+1, 35+6+5+3+1, 35+6+5+3+2+1, 35+6+5+4+1, 35+6+5+4+2+1, 35+7+1, 35+7+2+1, 35+7+3+1, 35+7+3+2+1, 35+7+4+1, 35+7+4+2+1, 35+8+7+1, 35+8+7+2+1, 35+8+7+3+1, 35+8+7+3+2+1, 35+8+7+4+1, 35+8+7+4+2+1, 35+9+7+1, 35+9+7+2+1, 35+9+7+3+1, 35+9+7+3+2+1, 35+9+7+4+1, 35+9+7+4+2+1, 35+10+7+1, 35+10+7+2+1, 35+10+7+3+1, 35+10+7+3+2+1, 35+10+7+4+1, 35+10+7+4+2+1, 35+11+1, 35+11+2+1, 35+11+3+1, 35+11+3+2+1, 35+11+4+1, 35+11+4+2+1, 35+12+11+1, 35+12+11+2+1, 35+12+11+3+1, 35+12+11+3+2+1, 35+12+11+4+1, 35+12+11+4+2+1, 35+13+11+1, 35+13+11+2+1, 35+13+11+3+1, 35+13+11+3+2+1, 35+13+11+4+1, 35+13+11+4+2+1, 35+14+11+1, 35+14+11+2+1, 35+14+11+3+1, 35+14+11+3+2+1, 35+14+11+4+1, 35+14+11+4+2+1, 35+15+1, 35+15+2+1, 35+15+3+1, 35+15+3+2+1, 35+15+4+1, 35+15+4+2+1, 35+16+15+1, 35+16+15+2+1, 35+16+15+3+1, 35+16+15+3+2+1, 35+16+15+4+1, 35+16+15+4+2+1, 35+17+15+1, 35+17+15+2+1, 35+17+15+3+1, 35+17+15+3+2+1, 35+17+15+4+1, 35+17+15+4+2+1, 35+18+15+1, 35+18+15+2+1, 35+18+15+3+1, 35+18+15+3+2+1, 35+18+15+4+1, 35+18+15+4+2+1, 35+19+1, 35+19+2+1, 35+19+3+1, 35+19+3+2+1, 35+19+4+1, 35+19+4+2+1, 35+20+19+1, 35+20+19+2+1, 35+20+19+3+1, 35+20+19+3+2+1, 35+20+19+4+1, 35+20+19+4+2+1, 35+21+19+1, 35+21+19+2+1, 35+21+19+3+1, 35+21+19+3+2+1, 35+21+19+4+1, 35+21+19+4+2+1, 35+22+19+1, 35+22+19+2+1,35+22+19+3+1,35+22+19+3+2+1,35+22+19+4+1,35+22+19+4+2+1,35+23+1,35+23+2+1, 35+23+3+1, 35+23+3+2+1, 35+23+4+1, 35+23+4+2+1, 35+24+23+1, 35+24+23+2+1, 35+24+23+3+1, 35+24+23+3+2+1, 35+24+23+4+1, 35+24+23+4+2+1, 35+25+23+1, 35+25+23+2+1, 35+25+23+3+1, 35+25+23+3+2+1, 35+25+23+4+1, 35+25+23+4+2+1, 35+26+1, 35+26+3+1, 35+26+3+2+1, 35+26+4+1, 35+26+4+2+1,35+27+26+1,35+27+26+3+1,35+27+26+3+2+1,35+27+26+4+1,35+27+26+4+2+1,35+28+1, 35+28+2+1, 35+29+28+1, 35+29+28+2+1, 35+30+28+1, 35+30+28+2+1, 35+31+1, 35+31+2+1, 35+32+31+1, 35+32+31+2+1, 35+33+31+1, 35+33+31+2+1, 35+34+31+1, 35+34+31+2+1, 36+1, 36+2+1, 37+1, 37+2+1, 37+3+1,37+3+2+1,37+4+1,37+4+2+1,37+5+1,37+5+2+1,37+5+3+1,37+5+3+2+1,37+5+4+1,37+5+4+2+1, 37+6+5+1, 37+6+5+2+1, 37+6+5+3+1, 37+6+5+3+2+1, 37+6+5+4+1, 37+6+5+4+2+1, 37+7+1, 37+7+2+1, 37+7+3+1, 37+7+3+2+1, 37+7+4+1, 37+7+4+2+1, 37+8+7+1, 37+8+7+2+1, 37+8+7+3+1, 37+8+7+3+2+1, 37+8+7+4+1, 37+8+7+4+2+1, 37+9+7+1, 37+9+7+2+1, 37+9+7+3+1, 37+9+7+3+2+1, 37+9+7+4+1, 37+9+7+4+2+1,37+10+7+1,37+10+7+2+1,37+10+7+3+1,37+10+7+3+2+1,37+10+7+4+1,37+10+7+4+2+1, 37+11+1, 37+11+2+1, 37+11+3+1, 37+11+3+2+1, 37+11+4+1, 37+11+4+2+1, 37+12+11+1, 37+12+11+2+1, 37+12+11+3+1, 37+12+11+3+2+1, 37+12+11+4+1, 37+12+11+4+2+1, 37+13+11+1, 37+13+11+2+1, 37+13+11+3+1, 37+13+11+3+2+1, 37+13+11+4+1, 37+13+11+4+2+1, 37+14+11+1, 37+14+11+2+1, 37+14+11+3+1, 37+14+11+3+2+1, 37+14+11+4+1, 37+14+11+4+2+1, 37+15+1, 37+15+2+1, 37+15+3+1, 37+15+3+2+1,37+15+4+1,37+15+4+2+1,37+16+15+1,37+16+15+2+1,37+16+15+3+1,37+16+15+3+2+1, 37+16+15+4+1, 37+16+15+4+2+1, 37+17+15+1, 37+17+15+2+1, 37+17+15+3+1, 37+17+15+3+2+1, 37+17+15+4+1, 37+17+15+4+2+1, 37+18+15+1, 37+18+15+2+1, 37+18+15+3+1, 37+18+15+3+2+1, 37+18+15+4+1, 37+18+15+4+2+1, 37+19+1, 37+19+2+1, 37+19+3+1, 37+19+3+2+1, 37+19+4+1, 37+19+4+2+1, 37+20+19+1, 37+20+19+2+1, 37+20+19+3+1, 37+20+19+3+2+1, 37+20+19+4+1, 37+20+19+4+2+1, 37+21+19+1, 37+21+19+2+1, 37+21+19+3+1, 37+21+19+3+2+1, 37+21+19+4+1, 37+21+19+4+2+1, 37+22+19+1, 37+22+19+2+1, 37+22+19+3+1, 37+22+19+3+2+1, 37+22+19+4+1, 37+22+19+4+2+1, 37+23+1, 37+23+2+1, 37+23+3+1, 37+23+3+2+1, 37+23+4+1, 37+23+4+2+1, 37+24+23+1, 37+24+23+2+1, 37+24+23+3+1, 37+24+23+3+2+1, 37+24+23+4+1, 37+24+23+4+2+1, 37+25+23+1, 37+25+23+2+1, 37+25+23+3+1, 37+25+23+3+2+1, 37+25+23+4+1, 37+25+23+4+2+1, 37+26+1, 37+26+3+1, 37+26+3+2+1, 37+26+4+1, 37+26+4+2+1, 37+27+26+1, 37+27+26+3+1, 37+27+26+3+2+1, 37+27+26+4+1, 37+27+26+4+2+1, 37+28+1, 37+28+2+1, 37+29+28+1, 37+29+28+2+1, 37+30+28+1, 37+30+28+2+1, 37+31+1, 37+31+2+1, 37+32+31+1, 37+32+31+2+1, 37+33+31+1, 37+33+31+2+1, 37+34+31+1 and 37+34+31+2+1.

In the list above, the numbers refer to the embodiments according to their numbering provided hereinabove whereas “+” indicates the dependency from another embodiment. The different individualized embodiments are separated by commas. In other words, “4+2+1” for example refers to embodiment 4) depending on embodiment 2), depending on embodiment 1), i.e. embodiment “4+2+1” corresponds to embodiment 1) further limited by the features of embodiments 2) and 4). Likewise, “10+7+2+1” refers to embodiment 10) depending mutatis mutandis on embodiments 7) and 2) and further depending on embodiment 1), i.e. embodiment “10+7+2+1” corresponds to embodiment 1) further limited by the features of embodiments 2) and further limited by the features of embodiments 7) and 10).

The compounds of formula I can be manufactured in accordance with the present invention using the procedures described hereafter.

Preparation of the Compounds of Formula I

### Abbreviations

The following abbreviations are used throughout the specification and the examples:
Ac acetyl
AcOH acetic acid
aq. aqueous
Boc tert-butyloxycarbonyl
Bn benzyl
Bu n-butyl
CC column chromatography over silica gel
Cipro ciprofloxacin
Cy cyclohexyl
DAD diode array detection
dba dibenzylideneacetone
DCC dicyclohexylcarbodiimide
DCM dichloromethane
DEA diethylamine
DIBAH diisobutylaluminium hydride
DIPEA diisopropylethylamine
DME 1,2-dimethoxyethane
DMF N,N-dimethylformamide
DMSO dimethylsulfoxide
DSC disuccinimidyl carbonate
EA ethyl acetate
EDC N-(3-dimethylaminopropyl)-N′-ethylcarbodiimide hydrochloride
ELSD evaporative light scattering detector
ESI electron spray ionisation
Et ethyl
Et₂O diethyl ether
EtOH ethanol
h hour(s)
HATU O-(7-azabenzotriazol-1-yl)-N,N,N′,N′-tetramethyluronium hexafluorophosphate
Hept heptane
Hex hexane
HMPA hexamethylphosphoramide
HOBT hydroxybenzotriazole
HPLC high performance liquid chromatography
iPr iso-propyl
IT internal temperature
LC-MS liquid chromatrography—mass spectrometry
LiHMDS lithium hexamethyldisilazide
MCPBA meta-chloroperbenzoic acid
Me methyl
MeCN acetonitrile
MeOH methanol
min minute(s)
MOM methoxymethyl
MS mass spectroscopy
Ms methylsulfonyl (mesyl)
NBS N-bromosuccinimide
NMR Nuclear Magnetic Resonance
NMP N-methyl-2-pyrrolidone
org. organic
Pd/C palladium on carbon
PE petroleum ether
PEPPSI™-IPr [1,3-bis(2,6-diisopropylphenyl)imidazol-2-ylidene](3-chloropyridyl)palladium(II) dichloride
Ph phenyl
PPTS para-toluenesulfonic acid pyridinium salt
prep-HPLC preparative HPLC
Pyr pyridine
rt room temperature
sat. saturated
TBAF tetra-n-butylammonium fluoride
TBDPS tert-butyldiphenylsilyl
TBME tert-butylmethylether
tBu tert-butyl
TEA triethylamine
Tf trifluoromethylsulfonyl (triflyl)
TFA trifluoroacetic acid
THF tetrahydrofuran
THP tetrahydropyranyl
TLC thin layer chromatography
TMS trimethylsilyl
TMSE 2-(trimethylsilyl)ethyl
Tr triphenylmethyl (trityl)
t_{R} retention time
Ts para-toluenesulfonyl
wt % percent in weight

General Reaction Techniques:

General reaction technique 1 (hydroxamic acid protecting group removal):

The protecting groups R of the hydroxamic acid ester derivatives (CONHOR) are removed as follows:
When R is THP, (2-methylpropoxy)ethyl, methoxymethyl, tBu, COOtBu or COtBu: by acidic treatment with e.g. TFA or HCl in an org. solvent such as DCM, dioxane, Et₂O or MeOH between 0° C. and rt or by treatment with PPTS in EtOH between rt and 80° C.;
When R is trityl: by treatment with diluted acid such as citric acid or HCl in an org. solvent such as MeOH or DCM;
When R is benzyl: by hydrogenolysis using general reaction technique 5;
When R is TMSE: by using fluoride anion sources such as BF₃.etherate complex in MeCN at 0° C., TBAF in THF between 0° C. and +40° C. or HF in MeCN or water between 0° C. and +40° C., or using acidic conditions such as AcOH in THF/MeOH or HCl in MeOH;
When R is allyl: by treatment with Pd(PPh₃)₄ in a solvent such as MeOH in the presence of K₂CO₃ or a scavenger such as dimedone, morpholine or tributyltin hydride;
When R is COMe: by treatment with diluted NaOH or Na₂CO₃ in a solvent such as MeOH.

Further general methods to remove hydroxamic acid protecting groups have been described in T. W. Greene & P. G. M. Wuts, Protecting Groups in Organic Synthesis, 3^{rd} Ed (1999), 23-147 (Publisher: John Wiley and Sons, Inc., New York, N.Y.).

General Reaction Technique 2 (Amide Coupling)

The carboxylic acid is reacted with the hydroxylamine derivative in the presence of an activating agent such as DCC, EDC, HOBT, n-propylphosphonic cyclic anhydride, HATU or DSC, in a dry aprotic solvent such as DCM, MeCN or DMF between −20° C. and 60° C. (see G. Benz in Comprehensive Organic Synthesis, B. M. Trost, I. Fleming, Eds; Pergamon Press: New York (1991), vol. 6, p. 381). Alternatively, the carboxylic acid can be activated by conversion into its corresponding acid chloride, by reaction with oxalyl chloride or thionyl chloride neat or in a solvent like DCM between −20° and 60° C. Further activating agents can be found in R. C. Larock, Comprehensive Organic Transformations. A guide to Functional Group Preparations, 2^{nd} Edition (1999), section nitriles, carboxylic acids and derivatives, p. 1941-1949 (Wiley-VC; New York, Chichester, Weinheim, Brisbane, Singapore, Toronto).

General Reaction Technique 3 (Alkyne-Haloalkyle Cross Coupling)

An alkyne-haloalkyne cross coupling reaction can be performed using a catalytic amount of copper derivative in the presence of aqueous hydroxylamine and a base such as piperidine or pyrrolidine (see Chodkiewicz and Cadiot, C. R. Hebd. Seances Acad. Sci. (1955), 241, 1055-1057), or in the presence of a ligand such as PPh₃ and a base such as K₂CO₃ in EtOH at reflux (see Wang et al., Synthesis (2011), 10, 1541-1546).

General Reaction Technique 4 (Stille Coupling):

The aromatic bromide is reacted with an ethynyl stanne derivative under Stille coupling conditions (as described in Echavarren and Stille, J. Am. Chem. Soc. (1987), 109, 5478-5486). Typical reaction conditions involve a palladium salt such as tetrakis(triphenylphosphine) palladium or dichloro bis(triphenylphophine) palladium, LiCl and a radical scavenger such as 2,6 dimethyl-4-methyl phenol in a solvent such as DMF or dioxane at a temperature ranging between 0° C. and 100° C., more preferably at a temperature ranging between 20° C. and 80° C.

General Reaction Technique 5 (Hydrogenolysis of a Benzyl Protecting Group):

The benzyl protected hydroxamic acid, dissolved in a solvent such as MeOH, EA or THF, is cleaved under hydrogen atmosphere in presence of a noble metal catalyst such as Pd/C or PtO₂, or Raney Ni. At the end of the reaction the catalyst is filtered off and the filtrate is evaporated under reduced pressure. Alternatively, the reduction can be performed by catalytic transfer hydrogenation using Pd/C and ammonium formate as hydrogen source.

General Reaction Technique 6 (Transformation of an Ester into an Acid):

When the ester side chain is a linear alkyl, the hydrolysis is usually performed by treatment with an alkali hydroxide such as LiOH, KOH or NaOH in a water-dioxane or water-THF mixture between 0° C. and 80° C. When the ester side chain is tBu, the release of the corresponding acid can also be performed in neat TFA or diluted TFA or HCl in an org. solvent such as ether or THF. When the ester side chain is the allyl group, the reaction is performed in the presence of tetrakis(triphenylphosphine)palladium(0) in the presence of an allyl cation scavenger such as morpholine, dimedone or tributyltin hydride between 0° C. and 50° C. in a solvent such as THF. When the ester side chain is benzyl, the reaction is performed under hydrogen in the presence of a noble metal catalyst such as Pd/C in a solvent such as MeOH, THF or EA. Further strategies to introduce other acid protecting groups and general methods to remove them have been described in T. W. Greene & P. G. M. Wuts, Protecting Groups in Organic Synthesis, 3^{rd} Ed. (1999), 369-441 (Publisher: John Wiley and Sons, Inc., New York, N.Y.).

General Reaction Technique 7 (Alcohol Activation)

The alcohol is reacted with MsCl, TfCl or TsCl in the presence of a base such as TEA in a dry aprotic solvent such as Pyr, THF or DCM between −30° C. and +50° C. In the case of the triflate or mesylate, Tf₂O or Ms₂O can also be used.

General Preparation Methods:

Preparation of the Compounds of Formula I:

The compounds of formula I can be manufactured by the methods given below, by the methods given in the examples or by analogous methods. Optimum reaction conditions may vary with the particular reactants or solvents used, but such conditions can be determined by a person skilled in the art by routine optimisation procedures.

The sections hereafter describe general methods for preparing compounds of formula I. If not indicated otherwise, the generic groups R¹, M, M^{A}, M^{B}, M^{C}, M^{D}, M^{E} and M^{F} are as defined for formula I. General synthetic methods used repeatedly throughout the text below are referenced to and described in the above section entitled “General reaction techniques”. In some instances certain generic groups might be incompatible with the assembly illustrated in the procedures and schemes below and so will require the use of protecting groups. The use of protecting groups is well known in the art (see for example T. W. Greene and P. G. M. Wuts, Protective Groups in Organic Synthesis, 3^{rd} Ed. (1999), Wiley-Interscience).

The compounds of formula I wherein R¹ is H can be obtained by deprotecting a compound of formula II

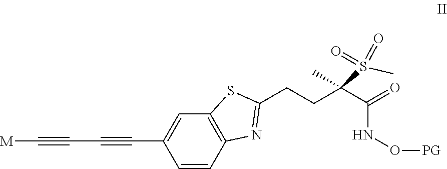

wherein M has the same meaning as in formula I and PG represents THP, TMSE, benzyl, trityl, (2-methylpropoxy)ethyl, methoxymethyl, allyl, tBu, acetyl, COOtBu or COtBu using general reaction technique 1. The reaction can also be performed with racemic material and the (R) enantiomer can be obtained by chiral HPLC separation.

The compounds of formula I wherein R¹ is not H can be obtained by:
a) reacting a compound of formula I wherein R¹ is H and M is as defined in formula I with a compound of formula III

(PG^{A}O)₂P—N(iPr)₂ III
wherein PG^{A} represents tert-butyl, the reaction being performed in the presence of a base such as tetrazole in a solvent such as acetonitrile at a temperature in the vicinity of 0° C., an oxidation reaction being subsequently performed adding an oxidizing agent such as hydrogen peroxide in water or MCPBA and subsequent cleavage of PG^{A} being performed using general reaction technique 1 (this reaction sequence can also be performed with racemic compound of formula I wherein R¹ is H and the (R)-enantiomer can then be obtained by chiral HPLC separation of the reaction products), whereby functional groups (e.g. amino or hydroxy) present on M that would be incompatible with the abovementioned reaction conditions can be protected (as carbamates or THP/silyl/tert-butyl ethers respectively) before performing said reaction and deprotected after performing said reaction, yielding compounds of formula I wherein R¹ is PO₃H₂; or
b) reacting a compound of formula I wherein R¹ is H and M is as defined in formula I with a compound of formula IV

HO(O)CR² IV
wherein R² is as defined in formula I, the reaction being performed using general reaction technique 2 (this reaction sequence can also be performed with racemic compound of formula I wherein R¹ is H and the (R)-enantiomer can then be obtained by chiral HPLC separation of the reaction product), whereby functional groups (e.g. amino or hydroxy) present on M that would be incompatible with the abovementioned reaction conditions can be protected (as carbamates or THP/silyl/tert-butyl ethers respectively) before performing said reaction and deprotected after performing said reaction, yielding compounds of formula I wherein R¹ is C(O)R²; or
c) reacting a compound of formula I wherein R¹ is H and M is as defined in formula I with a compound of formula V

X^{a}—(CH₂)—O—P(O)(OPG^{A})₂ V
wherein X^{a} represents iodine, bromine or chlorine and PG^{A} represents tert-butyl, the reaction being performed in the presence of a mineral base such as NaH or K₂CO₃ or in the presence of an organic base such as TEA or DIPEA in a solvent such as THF at a temperature ranging between −50° C. and rt and subsequent cleavage of PG^{A} being performed using general reaction technique 1 (this reaction sequence can also be performed with racemic compound of formula I wherein R¹ is H and the (R)-enantiomer can then be obtained by chiral HPLC separation of the reaction products), whereby functional groups (e.g. amino or hydroxy) present on M that would be incompatible with the abovementioned reaction conditions can be protected (as carbamates or THP/silyl/tert-butyl ethers respectively) before performing said reaction and deprotected after performing said reaction, yielding compounds of formula I wherein R¹ is CH₂—O—PO₃H₂; or
d) reacting a compound of formula I wherein R¹ is H and M is as defined in formula I with Pyr. SO₃ complex or Me₂NCHO. SO₃ complex in a solvent such as DMF or Pyr (this reaction sequence can also be performed with racemic compound of formula I wherein R¹ is H and the (R)-enantiomer can then be obtained by chiral HPLC separation of the reaction product), whereby functional groups (e.g. amino or hydroxy) present on M that would be incompatible with the abovementioned reaction conditions can be protected (as carbamates or THP/silyl/tert-butyl ethers respectively) before performing said reaction and deprotected after performing said reaction, yielding compounds of formula I wherein R¹ is SO₃H.

If desired, the compounds of formula I thus obtained may be converted into their salts, and notably into their pharmaceutically acceptable salts using standard methods.

Besides, whenever the compounds of formula I are obtained in the form of mixtures of enantiomers, the enantiomers can be separated using methods known to one skilled in the art, e.g. by formation and separation of diastereomeric salts or by HPLC over a chiral stationary phase such as a Regis Whelk-O1(R,R) (10 μm) column, a Daicel ChiralCel OD-H (5-10 μm) column, or a Daicel ChiralPak IA (10 μm) or AD-H (5 μm) column. Typical conditions of chiral HPLC are an isocratic mixture of eluent A (EtOH, in the presence or absence of an amine such as TEA or diethylamine) and eluent B (Hex), at a flow rate of 0.8 to 150 mL/min.

Preparation of the Compounds of Formula II:

The compounds of formula II can be obtained by:
a) reacting a compound of formula VI

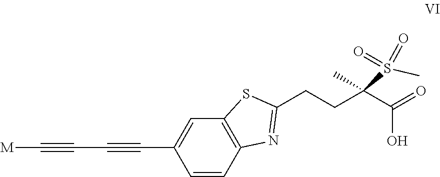

wherein M has the same meaning as in formula I with a compound of formula VII

H₂N-OPG IX
wherein PG has the same meaning as in formula II using general reaction technique 2 (this reaction can also be performed with racemic compound of formula VI and the (R)-enantiomer can then be obtained by chiral HPLC separation of the reaction products), whereby functional groups (e.g. amino or hydroxy) present on R^{1A} that would be incompatible with the coupling conditions mentioned in general reaction technique 2 can be protected (as carbamates or THP/silyl ethers respectively) before performing said reaction and deprotected after performing said reaction; or
b) reacting a compound of formula VIII

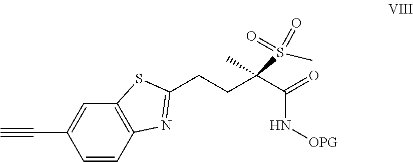

wherein PG has the same meaning as in formula II, with a compound of formula IX

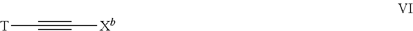

wherein T represents M and X^{b} represents iodine or bromine, using general reaction technique 3 (this reaction can also be performed with racemic compound of formula VIII and the (R)-enantiomer can then be obtained by chiral HPLC separation of the reaction products).

Preparation of the Synthesis Intermediates of Formulae VI, VII, VIII and IX:

Compounds of Formula VI:

The compounds of formula VI can be prepared as summarised in Scheme 1 hereafter.

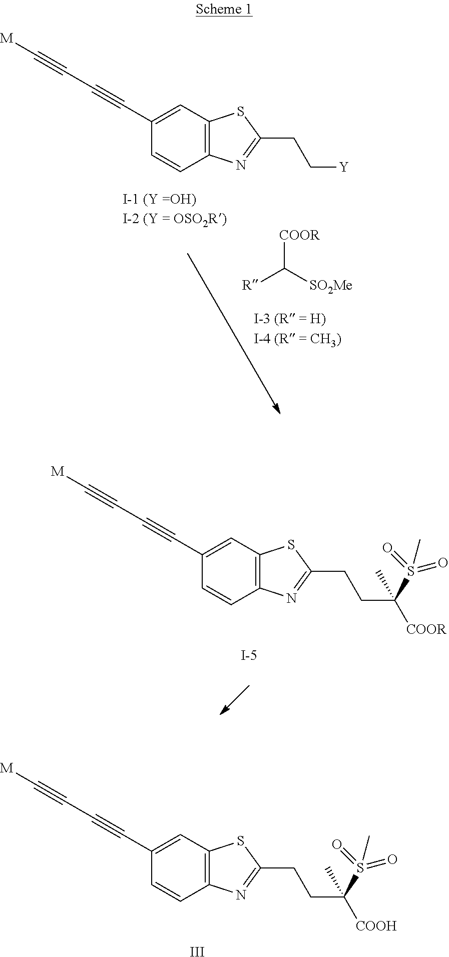

In Scheme 1, M has the same meaning as in formula I, R represents (C₁-C₅)alkyl, allyl or benzyl and R′ represents CH₃, CF₃ or tolyl. The reactions can also be performed with racemic material and the (R)-enantiomer can be obtained by chiral HPLC separation at any step when suitable.

The alcohols of formula I-1 can be transformed to the compounds of formula I-2 using general reaction technique 7. The compounds of formula I-2 can be reacted either with a 2-(methylsulfonyl)acetate derivative of formula I-3 in the presence of NaH, followed by alkylation with MeI in the presence of NaH, or directly with a 2-(methylsulfonyl)propanoate derivative of formula I-4 in the presence of NaH, affording the compounds of formula I-5. The compounds of formula I-5 can then be transformed into the carboxylic acid derivatives of formula VI using general reaction technique 6.

Compounds of Formula VII.

The compounds of formula VII are commercially available (PG=THP, tBu, COOtBu, Bn, TMSE, Tr, Ac, MOM or allyl) or can be prepared according to WO 2010/060785 (PG=(2-methylpropoxy)ethyl) or Marmer and Maerker, J. Org. Chem. (1972), 37, 3520-3523 (PG=COtBu).

Compounds of Formula VIII.

The compounds of formula VIII can be prepared as summarised in Scheme 2 hereafter.

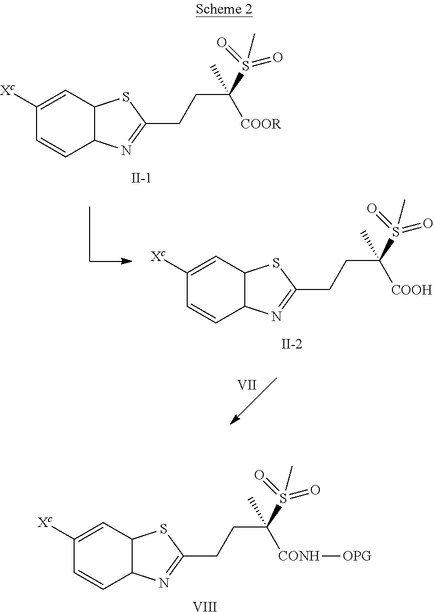

In Scheme 2, R represents (C₁-C₅)alkyl, allyl or benzyl, X^{c} represents bromine or ethynyl and PG has the same meaning as in formula II. The reactions can also be performed with racemic material and the (R)-enantiomer can be obtained by chiral HPLC separation at any step when suitable.

The derivatives of formula II-1 can be transformed into the carboxylic acid derivatives of formula II-2 using general reaction technique 6 and be further reacted with the compounds of formula VII using general reaction technique 2, thus affording the compounds of formula VIII wherein X^{c}=bromine or ethynyl. The derivatives of formula VIII wherein X^{c} represents bromine can be reacted with tributylethynyl stannane using general reaction technique 4 to afford the compound of formula VIII wherein X^{c} is ethynyl.

Compounds of Formula IX:

The compounds of formula IX wherein X^{b} represents iodine can be prepared from the corresponding compounds wherein X^{b} is H by treatment with iodine in the presence of an inorganic base such as KOH. The compounds of formula IX wherein X^{b} represents bromine can be prepared by reacting the corresponding compounds wherein X^{b} is H with NBS in presence of silver nitrate in a solvent such as acetone or acetonitrile.

Other Synthesis Intermediates and Starting Materials:

The compounds of formula II-1 wherein X^{c} represents bromine or ethynyl can be prepared as summarised in Scheme 3 hereafter.

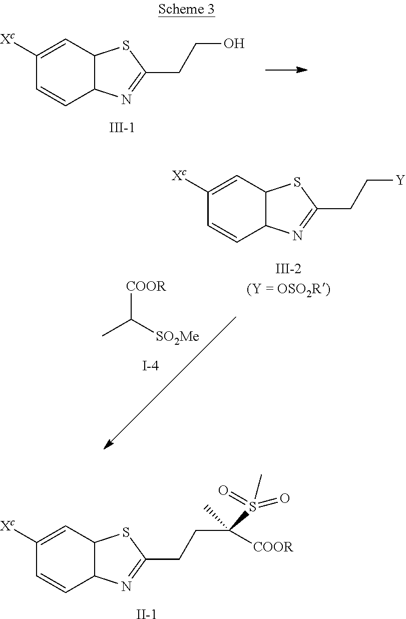

In Scheme 3, R represents (C₁-C₅)alkyl, allyl or benzyl, R′ represents CH₃, CF₃ or tolyl and X^{c} represents bromine or ethynyl. The reactions can also be performed with racemic material and the (R)-enantiomer can be obtained by chiral HPLC separation at any step when suitable.

The alcohols of formula III-1 can be transformed into the derivatives of formula III-2 using general reaction technique 7. The compounds of formula III-2 can then be reacted with the compounds of formula I-4 in the presence of NaH, affording the compounds of formula II-1 wherein X^{c} represents bromine.

The compounds of formula II-1 wherein X^{c} represents an ethynyl group can be prepared from the compounds of formula II-1 wherein X^{c} represents bromine applying general reaction technique 4.

The compound of formula III-1 wherein X^{c} represents bromine is commercially available or can be prepared by standard methods known to one skilled in the art.

The compounds of formula I-1 wherein M has the same meaning as in formula I can be prepared from compounds of formula III-1 wherein X^{c} represents bromine using general reaction technique 4, and then general reaction technique 3 using the appropriate compounds of formula IX as previously described.

Particular embodiments of the invention are described in the following Examples, which serve to illustrate the invention in more detail without limiting its scope in any way.

### EXAMPLES

All temperatures are stated in ° C. Unless otherwise indicated, the reactions take place at rt under inert atmosphere (nitrogen stream). The combined org. layers resulting from the workup of an aq. layer are, unless otherwise indicated, washed with a minimal volume of brine, dried over MgSO₄, filtered and evaporated to dryness to provide a so-called evaporation residue.

Analytical TLC characterisations were performed with 0.2 mm plates: Merck, Silica gel 60 F₂₅₄. Elution is performed with EA, Hept, DCM, MeOH or mixtures thereof. Detection was done with UV or with a solution of KMnO₄ (3 g), K₂CO₃ (20 g), 5% NaOH (3 mL) and H₂O (300 mL) with subsequent heating.

CCs were performed using Brunschwig 60 A silica gel (0.032-0.63 mm) or using an ISCO CombiFlash system and prepacked SiO₂ cartridges, elution being carried out with either Hept-EA or DCM-MeOH mixtures with an appropriate gradient. When the compounds contained an acid function, 1% of AcOH was added to the eluent(s). When the compounds contained a basic function, 25% aq. NH₄OH was added to the eluents.

The compounds were characterized by ¹H NMR (300 MHz, Varian Oxford; 400 MHz, Bruker Avance 400 or 500 MHz, Bruker Avance 500 Cryoprobe). Chemical shifts 6 are given in ppm relative to the solvent used; multiplicities: s=singlet, d=doublet, t=triplet, q=quartet, p=pentet, hex=hexet, hep=Heptet, m=multiplet, br.=broad; coupling constants J are given in Hz. Alternatively compounds were characterized by LC-MS (Sciex API 2000 with Agilent 1100 Binary Pump with DAD and ELSD or an Agilent quadrupole MS 6140 with Agilent 1200 Binary Pump, DAD and ELSD); by TLC (TLC plates from Merck, Silica gel 60 F254); or by melting point.

The analytical LC-MS data have been obtained using the following respective conditions:
Column: Zorbax SB-Aq, 30.5 μm, 4.6×50 mm;
Injection volume: 1 μL;
Column oven temperature: 40° C.;
Detection: UV 210 nm, ELSD and MS;
MS ionization mode: ESI+;
Eluents: A: H₂O+0.04% TFA; and B: MeCN;
Flow rate: 40.5 mL/min;
Gradient: 5% B to 95% B (0.0 min-1.0 min), 95% B (1.0 min-1.45 min).

The number of decimals given for the corresponding [M+H⁺] peak(s) of each tested compound depends upon the accuracy of the LC-MS device actually used.

The prep-HPLC purifications were performed on a Gilson HPLC system, equipped with a Gilson 215 autosampler, Gilson 333/334 pumps, Dionex MSQ Plus detector system, and a Dionex UVD340U (or Dionex DAD-3000) UV detector, using the following respective conditions:
Method 1:
   Column: Waters XBridge C18, 10 μm, 30×75 mm;
   Flow rate: 75 mL/min;
   Eluents: A: H₂O+0.1% HCOOH; B: MeCN+0.1% HCOOH;
   Gradient: 70% A to 5% A (0.0 min-3.5 min), 5% A (3.5 min-6.0 min).
Method 2:
   Column: Waters XBridge C18, 10 μm, 30×75 mm;
   Flow rate: 75 mL/min;
   Eluents: A: H₂O+0.5% aq. NH₄OH 25% solution; B: MeCN;
   Gradient: 90% A to 5% A (0.0 min-4.0 min), 5% A (4.0 min-6.0 min).

Besides, semi-preparative chiral HPLCs were performed using the conditions hereafter.

Semi-Preparative Chiral HPLC Method A:

The semi-preparative chiral HPLC is performed on a Daicel ChiralPak ID column (30×250 mm, 5 μM) using the eluent mixture, flow rate and detection conditions indicated between brackets in the corresponding experimental protocol. The retention times are obtained by elution of analytical samples on a Daicel ChiralPak ID column (4.6×250 mm, 5 μM) using the same eluent mixture with the flow rate indicated between brackets in the corresponding experimental protocol.

Procedures.

Procedure A:

CuCl (0.0117 g; 0.118 mmol) and NH₂OH.HCl (0.0833 g, 1.2 mmol) are dissolved in BuNH₂ (30% in water, 0.75 mL). The terminal alkyne (0.250 g; 0.59 mmol) and BuNH₂ (0.288 mL, 2.32 mmol) is added. The reaction mixture is ice-chilled and halo-alkyne (0.157 g; 0.768 mmol) in dioxane (0.1 mL) is added at 0° C. The reaction proceeds 1 h at that temperature. The reaction mixture is then allowed to warm up to rt over 1 h. Water (5 mL) and EA (30 mL) are added and two phases are separated. The aq. layer is extracted with EA (10 mL). The evaporation residue is then purified by CC or by prep-HPLC using a suitable method to afford the bis-alkyne product.

Procedure B:

To a solution of the THP-protected hydroxamic acid derivative (0.15 mmol) in MeOH (1.2 mL) and water (0.4 mL) is added 2M aq. HCl (0.6 mL; 1.2 mmol). The reaction mixture is stirred at rt until completion. The reaction mixture, after neutralization with sat. aq. NaHCO₃ is extracted with DCM-MeOH (9-1, 3×20 mL). The evaporation residue is then purified by CC (DCM-MeOH) or by prep-HPLC using a suitable method.

Procedure C:

To the THP-protected hydroxamic acid derivative (0.02 mmol) in EtOH (3 mL) is added PPTS (0.025 g; 0.03 mmol). The mixture is stirred at 80° C. for 2 h, cooled to rt and directly purified by CC (DCM-MeOH) or by prep-HPLC using a suitable method.

Procedure D:

A solution of the THP-protected hydroxamic acid derivative (0.090 g; 0.12 mmol) in 4M HCl in dioxane (1 mL) is stirred 10 min at rt. The mixture is directly purified by prep-HPLC using a suitable method.

### PREPARATIONS

### Preparation A: (2R)-4-(6-bromobenzo[d]thiazol-2-yl)-2-methyl-2-(methylsulfonyl)-N-(((2RS)-tetrahydro-2H-pyran-2-yl)oxy)butanamide

### A.i. 2-(6-bromobenzo[d]thiazol-2-yl)ethyl methanesulfonate

To an ice-chilled solution of 2-(6-bromobenzo[d]thiazol-2-yl)ethanol (10.2 g; 39.5 mmol, prepared as described in US 2004/224953) in DCM (80 mL) was added dropwise TEA (11.7 mL, 84.2 mmol) and MsCl (5.64 mL, 72.5 mmol). The mixture was stirred at 0° C. for 10 min. The mixture was diluted with a sat. NaHCO₃ solution (100 mL), extracted with DCM (100 mL) and the org. layer was washed with brine (100 mL), dried over MgSO₄ and concentrated to dryness to afford the title product as a yellow solid (12 g; 90% yield).

¹H NMR (d6-DMSO) δ: 8.39 (d, J=1.8 Hz, 1H); 7.91 (d, J=8.7 Hz, 1H); 7.66 (dd, J=1.8, 8.7 Hz, 1H); 4.66 (t, J=6.1 Hz, 3H); 3.57 (t, J=6.1 Hz, 3H).

MS (ESI, m/z): 335.9 [M+H⁺] for C₁₀H₁₀NO₃BrS₂; t_{R}=0.82 min.

### A.ii. Rac-ethyl 4-(6-bromobenzo[d]thiazol-2-yl)-2-methyl-2-(methylsulfonyl)butanoate

To a solution of ethyl 2-(methylsulfonyl)propanoate (4.3 g; 23.7 mmol, commercial) in DMF (26 mL) was added portionwise NaH (0.9 g; 22.5 mmol). The mixture was stirred at 0° C. for 15 min and was allowed to reach 10° C. Then, a solution of intermediate A.i (7.58 g; 22.5 mmol) in DMF (26 mL) was added dropwise. The mixture was stirred at 10° C. for 30 min. EA (100 mL) was added and the mixture was poured into 10% aq. NaHSO₄ (100 mL). The org. layer was then washed with water (100 mL), brine (100 mL), dried over MgSO₄ and concentrated to dryness. The residue was purified by CC (Hept-EA) to afford the title compound as a pale yellow solid (5.46 g; 58% yield).

¹H NMR (d6-DMSO) δ: 8.38 (d, J=2.0 Hz, 1H); 7.89 (d, J=8.7 Hz, 1H); 7.65 (dd, J=2.0, 8.6 Hz, 1H); 4.18 (q, J=7.1 Hz, 2H); 3.28-3.33 (overlapped m, 1H); 3.15 (s, 3H); 3.07-3.11 (m, 1H); 2.66-2.75 (m, 1H); 2.31-2.40 (m, 1H); 1.60 (s, 3H); 1.21 (t, J=7.1 Hz, 3H).

MS (ESI, m/z): 422.0 [M+H⁺] for C₁₅H₈NO₄BrS₂₂; t_{R}=0.89 min.

### A.iii. (2R)-ethyl 4-(6-bromobenzo[d]thiazol-2-yl)-2-methyl-2-(methylsulfonyl)butanoate

Intermediate A.ii (8.42 g) was separated by semi-preparative chiral HPLC Method A (MeOH-DEA-DCM 74.92-0.08-25; flow rate: 16 mL/min; UV detection at 227 nM); the respective retention times (flow rate: 0.8 mL/min) were 5.45 and 6.17 min. The title (R)-enantiomer was identified as the second-eluting enantiomer and was obtained as a yellow solid (4 g).

¹H NMR (d6-DMSO) δ: 8.38 (d, J=2.0 Hz, 1H); 7.89 (d, J=8.7 Hz, 1H); 7.65 (dd, J=2.0, 8.6 Hz, 1H); 4.18 (q, J=7.1 Hz, 2H); 3.28-3.33 (overlapped m, 1H); 3.15 (s, 3H); 3.07-3.11 (m, 1H); 2.66-2.75 (m, 1H); 2.31-2.40 (m, 1H); 1.60 (s, 3H); 1.21 (t, J=7.1 Hz, 3H).

MS (ESI, m/z): 419.8 [M+H⁺] for C₁₅H₈NO₄BrS₂; t_{R}=0.90 min.

### A.iv. (2R)-4-(6-bromobenzo[d]thiazol-2-yl)-2-methyl-2-(methylsulfonyl)butanoic acid lithium salt

To a solution of intermediate A.iii (16.4 g; 38.9 mmol) in MeOH (80 mL) and THF (80 mL) was added a solution of LiOH.H₂O (3.46 g; 82.5 mmol) in water (40 mL). The mixture was stirred at 50° C. for 1 h. The mixture was concentrated to dryness and dried to a constant weight to afford the title product as a yellow foam (16.9 g; >95% yield).

¹H NMR (d6-DMSO) δ: 8.34 (d, J=2 Hz, 1H); 7.87 (d, J=8.7 Hz, 1H); 7.62 (dd, J=2.0, 8.7 Hz, 1H); 3.13-3.20 (m, 2H); 3.08 (s, 3H); 2.50-2.58 (m, 1H); 2.06-2.18 (m, 1H); 1.40 (s, 3H).

MS (ESI, m/z): 391.9 [M+H⁺] for C₁₃H₁₅NO₄BrS₂; t_{R}=0.76 min.

### A.v. (2R)-4-(6-bromobenzo[d]thiazol-2-yl)-2-methyl-2-(methylsulfonyl)-N—(((RS)-tetrahydro-2H-pyran-2-yl)oxy)butanamide

To a solution of intermediate A.iv (12 g; 30.1 mmol) in DMF (120 mL) was added O-(tetrahydro-2H-pyran-2-yl)hydroxylamine (10.7 g; 91.6 mmol), EDC (17.6 g; 91.8 mmol), HOBT.H₂O (12.4 g; 91.8 mmol) and TEA (13 mL, 93.3 mmol). It was stirred at 40° C. for 24 h. The evaporation residue was purified by CC (Hept-EA) to afford the title compound as a yellow foam (9.83 g; 66% yield).

¹H NMR (d6-DMSO) δ: 11.39 (s, 1H); 8.37 (d, J=1.7 Hz, 1H); 7.89 (d, J=8.7 Hz, 1H); 7.65 (dd, J=2.0, 8.6 Hz, 1H); 4.96 (d, J=2.0 Hz, 1H); 4.11-3.98 (m, 2H); 3.54-3.45 (m, 1H); 3.07 (s, 1.5H); 3.05 (s, 1.5H); 3.04-2.91 (overlapped m, 1H); 2.84-2.68 (m, 1H); 2.33-2.19 (m, 1H); 1.65-1.47 (m, 9H).

MS (ESI, m/z): 491.4 [M+H⁺] for C₁₈H₂₃N₂O₅BrS₂; t_{R}=0.84 min.

### A.vi. (2R)-4-(6-ethynylbenzo[d]thiazol-2-yl)-2-methyl-2-(methylsulfonyl)-N—(((RS)-tetrahydro-2H-pyran-2-yl)oxy) butanamide

To a mixture of intermediate A.v (2 g; 4.07 mmol), cesium fluoride (1.233 g; 8.14 mmol) and bis(tri-tert-butylphosphine)palladium (0.152 g; 0.297 mmol) in degassed dioxane (20 mL) was added ethynyltributylstannane (1.77 mL, 6.1 mmol). The mixture was stirred at 80° C. for 10 min. The mixture was diluted with DCM (100 mL) and aq. sat. NaHCO₃ (100 mL). The evaporation residue was purified by CC (Hept-EA) to afford the title compound as a yellow foam (1.33 g; 75% yield).

¹H NMR (d6-DMSO) δ: 11.45-11.41 (m, 1H); 8.28-8.26 (m, 1H); 7.94 (d, J=8.4 Hz, 1H); 7.57 (dd, J=1.5, 8.4 Hz, 1H); 5.00-4.94 (m, 1H); 4.28 (s, 1H); 4.15-4.06 (m, 1H); 3.55-3.47 (m, 1H); 3.31-3.22 (overlapped m, 1H); 3.08 (s, 1.5H); 3.06 (s, 1.5H); 3.05-2.96 (m, 1H), 2.84-2.72 (m, 1H); 2.33-2.21 (m, 1H); 1.77-1.47 (m, 9H).

MS (ESI, m/z): 437.2 [M+H⁺] for C₂₀H₂₄N₂O₅S₂; t_{R}=0.82 min.

### Preparation B: 3-(bromoethynyl)azetidine hydrochloride

### B.i. Tert-butyl 3-(bromoethynyl)azetidine-1-carboxylate

To a solution of tert-butyl 3-ethynylazetidine-1-carboxylate (2.8 g; 15.4 mmol; prepared as described in WO 2014/165075) and NBS (3.3 g; 18.5 mmol) in acetone (60 mL) was added AgNO₃ (0.262 g; 1.54 mmol). The mixture was stirred at rt for 1.5 h. After filtration over Celite, the evaporation residue was purified by CC (Hex-TBME) to give the title compound as a yellowish oil (3.48 g; 87% yield).

¹H NMR (CDCl₃) δ: 4.14 (m, 2H); 3.96 (dd, J=6.3, 8.4 Hz, 2H); 3.34 (m, 1H); 1.46 (s, 9H).

### B.ii. 3-(bromoethynyl)azetidine hydrochloride

A solution of intermediate B.i (2 g; 7.69 mmol) in 4M HCl in dioxane (20 mL; 80 mmol) was stirred at rt for 1 h. The reaction mixture was concentrated to dryness then co-evaporated with Et₂O (2×10 mL) to give the title compound as a light yellow solid (1.49 g; >95% yield).

¹H NMR (CDCl₃) δ: 9.44-9.10 (m, 2H); 4.15-4.06 (m, 2H); 3.96-3.87 (m, 2H); 3.74 (m, 1H).

MS (ESI, m/z): 162.0 [M+H⁺] for C₅H₆NBr; t_{R}=0.23 min.

### Preparation C: 3-(bromoethynyl)-1-(oxetan-3-yl)azetidine

To a solution of the compound of Preparation B (0.58 g; 0.534 mmol) in DCM (39 mL) were added oxetan-3-one (0.639 g; 8.86 mmol) and NaBH(OAc)₃ (3.757 g; 17.7 mmol). The reaction mixture was stirred at rt for 1 h. Sat. aq. NaHCO₃ (50 mL) and DCM (60 mL) were added. The aq. layer was extracted with DCM (2×50 mL). The evaporation residue was purified by CC (DCM-MeOH) to afford the title compound as a white solid (0.53 g; 83% yield).

¹H NMR (CDCl₃) δ: 4.51 (t, J=6.6 Hz, 2H); 4.29 (dd, J=5.3, 6.3 Hz, 2H); 3.64 (m, 1H); 3.48-3.45 (m, 2H); 3.25 (m, 1H); 3.07-3.03 (m, 2H).

MS (ESI, m/z): 217.9 [M+H⁺] for C₈H₁₀NOBr; t_{R}=0.26 min.

### Preparation D: 3-iodoprop-2-yn-1-yl 4-hydroxypiperidine-1-carboxylate

### D.i. Prop-2-yn-1-yl 4-hydroxypiperidine-1-carboxylate

To an ice-chilled solution of propargyl chloroformate (2.47 mL; 24.3 mmol) in DCM (50 mL) was added 4-hydroxypiperidine (2.5 g; 24.3 mmol), followed by TEA (6.76 mL; 48.6 mmol). The reaction mixture was stirred overnight at rt. The reaction mixture was washed with aq. NaHCO₃ (3×100 mL) and brine (100 mL). The evaporation residue was purified by CC (DCM-TBME) to afford the title product as a beige oil (3.17 g; 71% yield).

¹H NMR (d6-DMSO) δ: 4.75 (d, J=4.1 Hz, 1H); 4.65 (d, J=2.4 Hz, 2H); 3.70-3.62 (m, 3H); 3.50 (m, 1H); 3.08-3.06 (m, 2H); 1.74-1.67 (m, 2H); 1.33-1.25 (m, 2H).

### D. ii. 3-iodoprop-2-yn-1-yl 4-hydroxypiperidine-1-carboxylate

To a solution of intermediate D.i (2.02 g; 11 mmol) in MeOH (49.2 mL) and aq. 1M KOH (55.1 mL; 55.1 mmol) was added in one portion iodine (3.6 g; 14.3 mmol). The reaction mixture was stirred overnight at rt. The solvent was evaporated and the residue was diluted with H₂O (400 mL) and extracted with DCM (2×500 mL). The evaporation residue was purified by CC (Hept-EA-MeOH) to afford the title compound as a white solid (2.32 g; 68% yield).

¹H NMR (d6-DMSO) δ: 4.77 (s, 2H); 4.75 (d, J=4.1 Hz, 1H); 3.75-3.61 (m, 3H); 2.97-3.13 (m, 2H); 1.74-1.64 (m, 2H); 1.36-1.21 (m, 2H).

MS (ESI, m/z): 309.9 [M+H⁺] for C₉H₁₂NO₃I; t_{R}=0.63 min.

### Preparation E: 3-bromoprop-2-yn-1-yl 3-hydroxyazetidine-1-carboxylate

### E.i. 3-bromoprop-2-yn-1-yl (2,5-dioxopyrrolidin-1-yl) carbonate

To a solution of 3-bromoprop-2-yn-1-ol (1 g; 7.4 mmol) in MeCN (85 mL) was added TEA (2.1 mL, 14.8 mmol) and DSC (6.0 g; 22.2 mmol). The reaction mixture was stirred at rt for 30 min. The reaction mixture was diluted with EtOAc (100 mL), washed with 5% aq. citric acid (3×50 mL), water (50 mL) and brine (50 mL). The evaporation residue was purified by CC (Hept-EA gradient) to afford the title product as a beige solid (1.38 g; 67% yield).

¹H NMR (d6-DMSO) δ: 5.13 (s, 2H); 2.83 (s, 4H).

### E.ii. 3-bromoprop-2-yn-1-yl 3-hydroxyazetidine-1-carboxylate

To a solution of intermediate E.i (1.38 g; 5 mmol) in DCM (65 mL) were added 3-hydroxyazetidine hydrochloride (0.56 g, 5 mmol) and TEA (1.4 mL, 10 mmol). The reaction mixture was stirred for 45 min at rt. The mixture was diluted in DCM (200 mL), washed with sat. aq. NaHCO₃ (3×200 mL) and brine (200 mL). The evaporation residue afforded the title product as a light grey solid (0.87 g; 75% yield).

¹H NMR (d6-DMSO) δ: 5.73 (d, J=6.6 Hz, 1H); 4.68 (s, 2H); 4.43 (m, 1H); 4.10 (d, J=1.5 Hz, 2H); 3.68-3.66 (m, 2H).

### Preparation F: 3-(bromoethynyl)-1-(2-((tert-butyldimethylsilyl)oxy)ethyl)azetidine

To a solution of the compound of Preparation B (0.38 g; 1.91 mmol) in DCM (20 mL) were added (tert-butyldimethylsilyloxy)acetaldehyde (1 mL; 5.25 mmol) and NaBH(OAc)₃ (2.39 g; 11.3 mmol). The reaction mixture was stirred overnight. Sat. aq. NaHCO₃ (30 mL) and DCM (10 mL) were added. The two layers were separated and the aq. layer was extracted with DCM (2×30 mL). The evaporation residue was purified by CC (Hept-EA) to afford the title compound as an orange oil (0.2 g; 33% yield).

¹H NMR (d6-DMSO) δ: 3.67-3.63 (m, 4H); 3.27 (t, J=7.7 Hz, 1H); 3.17-3.12 (m, 2H); 2.58 (t, J=5.7 Hz, 2H); 0.91 (s, 9H); 0.07 (s, 6H).

MS (ESI, m/z): 395.98 [M+H⁺] for C₁₅H₂₇NO₄BrP; t_{R}=0.64 min.

### Preparation G: 2-(3-(bromoethynyl)azetidin-1-yl)ethyl di-tert-butyl phosphate

### G.i. 2-(3-(Bromoethynyl)azetidin-1-yl)ethan-1-ol hydrochloride

To a solution of the compound of Preparation F (0.250 g; 0.785 mmol) in dioxane (0.5 mL) was added 4M HCl in dioxane (0.982 mL; 3.93 mmol. After stirring for 10 min, the solvent was removed in vacuo and the residue was co-evaporated twice with toluene to afford the title compound as a colourless oil (0.19 g; >95% yield).

¹H NMR (CDCl₃) δ: 3.61 (t, J=7.3 Hz, 2H); 3.53-3.51 (m, 2H); 3.25 (m, 1H); 3.14-3.11 (m, 2H); 2.96 (br. s, 1H); 2.61-2.57 (m, 2H).

### G. ii. 2-(3-(bromoethynyl)azetidin-1-yl)ethyl di-tert-butyl phosphate

To a solution of intermediate G.i (0.190 g; 0.79 mmol)) in THF (2 mL), cooled to 0° C., was added portionwise NaH (60% in oil dispersion, 0.079 g; 1.97 mmol). The mixture was stirred at 0° C. for 5 min and at for 45 min. After cooling to 0° C., di-tert-butyl phosphorochloridate (commercial, 0.253 g; 1.11 mmol) was added dropwise. The reaction mixture was stirred at 0° C. for 5 min, and stirred at rt for 5 h. EA (20 mL) and water (20 mL) were added. The two layers were separated and the aq. layer was extracted with EA (20 mL). The evaporation residue was purified by CC (DCM-MeOH containing 1% NH₄OH) to afford the title compound as a colourless oil (0.154 g; 49% yield).

¹H NMR (d6-DMSO) δ: 3.76 (q, J=5.9 Hz, 1H); 3.48 (t, J=6.9 Hz, 1H); 3.21 (m, 1H); 3.02 (t, J=6.6 Hz, 1H); 2.57 (t, J=5.5 Hz, 1H); 1.41 (s, 9H).

### Preparation H: ((1R,2R)-2-(bromoethynyl)cyclobutyl)methanol

### H.i. ((1S*,2S*)-2-(hydroxymethyl)cyclobutyl)methyl benzoate

To an ice-chilled solution of ((1S*,2S*)-cyclobutane-1,2-diyl)dimethanol (prepared as described in Jakovac et al., J. Am. Chem. Soc. (1982), 104, 4659-4665; 3.40 g; 29 mmol) in THF (150 mL) at 0° C. was added NaH (60% dispersion in oil; 1.081 g; 27 mmol) and the solution was stirred at rt for 30 min. Benzoyl chloride (3.14 mL, 27 mmol) was added and the mixture was stirred at rt overnight. The reaction mixture was partitioned between a solution of NH₄Cl (100 mL) and Et₂O (50 mL). The two layers were separated and the aq. layer was extracted with Et₂O (100 mL). The evaporation residue was purified by CC (Hept-EA) to afford the title compound as a colourless oil (4.35 g; 67% yield).

¹H NMR (d6-DMSO) δ: 7.99-7.97 (m, 2H); 7.67 (m, 1H); 7.55 (m, 2H); 4.49 (t, J=5.3 Hz, 1H); 4.26 (d, J=6.4 Hz, 2H); 3.41 (t, J=5.7 Hz, 2H); 2.46 (m, 1H); 2.25 (m, 1H); 1.97-1.84 (m, 2H); 1.76-1.62 (m, 2H).

MS (ESI, m/z): 221.1 [M+H⁺] for C₁₃H₁₆O₃; t_{R}=0.76 min.

### H.ii. ((1S,2S)-2-(hydroxymethyl)cyclobutyl)methyl benzoate and ((1R,2R)-2-(hydroxymethyl)cyclobutyl)methyl benzoate

Intermediate H.i (4.35 g) was separated by semi-preparative chiral HPLC Method A (CO₂-MeOH 90-10; flow rate: 160 mL/min; UV detection at 210 nM); the respective retention times were 2.8 and 3.4 min. The (S,S)-enantiomer, identified as the first eluting compound, was obtained as a colourless oil (1.89 g). The (R,R)-enantiomer was also obtained as a colourless oil (1.89 g).

The absolute stereochemistry of (1S,2S)-enantiomer was assigned comparing the HPLC retention time obtained with an authentic sample obtained from (1S,2S)-cyclobutane-1,2-dicarboxylic acid (Gryko et al., Tetrahedron: Asymmetry (2004), 15, 1103-1113). Analytical chiral HPLC Method A (CO₂-MeOH 85-15; flow rate: 4 mL/min; UV detection at 210 nm): t_{R}=1.53 min. (1R,2R)-enantiomer elutes at 1.80 min.

### H.iii. ((1S,2S)-2-formylcyclobutyl)methyl benzoate

To an ice-chilled solution of intermediate H.ii (1.89 g; 8.58 mmol) in DCM (50 mL) was added DIPEA (5.9 mL; 34.5 mmol). A solution of Pyr.SO₃ complex (3.23 g; 9.13 mmol) in DMSO (13.9 mL, 196 mmol) was added slowly over 30 min. The reaction was further stirred at 0° C. for 1 h. The reaction mixture was partitioned between water (25 mL) and DCM (150 mL). The two layers were separated and the aq. layer was extracted twice with DCM (2×50 mL). The evaporation residue was purified by CC (Hept-EA) to afford the title compound as a colourless oil (1.62 g; 87% yield).

¹H NMR (d6-DMSO) δ: 9.68 (d, J=1.7 Hz, 1H); 7.99-7.96 (m, 2H); 7.67 (m, 1H); 7.56-7.53 (m, 2H); 4.36-4.28 (m, 2H); 3.18 (m, 1H); 2.94 (m, 1H); 2.15-1.84 (m, 4H).

### H.iv. ((1S,2S)-2-(2,2-dibromovinyl)cyclobutyl)methyl benzoate

To a solution of CBr₄ (5.024 g; 14.8 mmol) in DCM (28 mL), cooled to −20° C., was added PPh₃ (8.112 g; 29.7 mmol) in DCM (42 mL). After 30 min, the reaction mixture was cooled to −78° C. and TEA (2.07 mL; 14.8 mmol) was added followed by intermediate H.iii (1.62 g; 7.42 mmol) in DCM (28 mL). After 1 h stirring at −78° C., the reaction mixture was warmed to rt. The reaction mixture was concentrated to about half its volume and Et₂O (100 mL) was added. The suspension was filtered and the evaporation residue was purified by CC (Hept-EA) to afford the title compound as a colourless oil (2.41 g; 87% yield).

¹H NMR (d6-DMSO) δ: 7.98-7.97 (m, 2H); 7.67 (m, 1H); 7.56-7.53 (m, 2H); 6.80 (d, J=8.9 Hz, 1H); 4.31-4.21 (m, 2H); 2.99 (quint, J=8.4 Hz, 1H); 2.70 (m, 1H); 2.09 (m, 1H); 1.94-1.85 (m, 2H); 1.78 (m, 1H).

### H.v. ((1S,2S)-2-(bromoethynyl)cyclobutyl)methyl benzoate

To a solution of intermediate H.iv (2.41 g; 6.44 mmol) in THF (8.5 mL) was added TBAF (1M in THF; 25.6 mL; 25.6 mmol) and the mixture was stirred at rt for 24 h. The solvent was removed in vacuo and the evaporation residue was purified by CC (Hept-EA) to afford the title compound as a colourless oil (1.50 g; 80% yield).

¹H NMR (d6-DMSO) δ: 8.00-7.98 (m, 2H); 7.68 (m, 1H); 7.54 (t, J=7.8 Hz, 2H); 4.33-4.21 (m, 2H); 2.97 (q, J=8.6 Hz, 1H); 2.76 (m, 1H); 2.15 (m, 1H); 1.98-1.88 (m, 2H); 1.77 (m, 1H).

### H.vi. ((1R,2R)-2-(bromoethynyl)cyclobutyl)methanol

To a solution of intermediate H.v (1.5 g; 5.12 mmol) in MeOH (15 mL) was added K₂CO₃ (1.414 g; 10.2 mmol). After 30 min, the reaction mixture was partitioned between DCM (100 mL) and 10% aq. NaHSO₄ (20 mL). The two layers were separated and the aq. layer was extracted with DCM (100 mL). The evaporation residue was purified by CC (Hept-EA) to afford the title compound as a colourless oil (0.968 g; >95% yield).

¹H NMR (d6-DMSO) δ: 4.60 (t, J=5.4 Hz, 1H); 3.38-3.33 (m, 2H); 2.81 (q, J=8.6 Hz, 1H); 2.40 (m, 1H); 2.07 (m, 1H); 1.90-1.64 (m, 3H).

### Preparation I: (S)-2-(bromoethynyl)azetidine

### I.i. Tert-butyl (2S)-2-(bromoethynyl)azetidine-1-carboxylate

Starting from tert-butyl (2S)-2-(hydroxymethyl)azetidine-1-carboxylate (commercial; 5.52 g; 29.5 mmol) and proceeding successively in analogy to Preparation H, steps H.iii to H.v, the title compound was obtained, after purification by CC (Hept-EA), as a colourless oil (3.51 g).

¹H NMR (d6-DMSO) δ: 4.76 (m, 1H); 3.83-3.66 (m, 2H); 2.46 (overlapped m, 1H); 2.18 (m, 1H); 1.38 (s, 9H).

### I.ii. (2S)-2-(bromoethynyl)azetidine

To a solution of intermediate I.i (0.3 g; 1.15 mmol) in MeCN (1.5 mL) was added a mixture of water (3 mL) and sulfuric acid (0.329 mL). The reaction was stirred at 60° C. for 1 h. The solution was cooled to rt, and 15% aq. NaOH was added until pH=7 was reached. The mixture was concentrated to dryness. The residue was triturated in a DCM-MeOH mixture (9-1; 30 mL) for 40 min, then filtered. The filtrate was concentrated to dryness to afford the title compound as an orange oil (0.153 g; 83% yield).

¹H NMR (d6-DMSO) δ: 4.97 (t, J=8.4 Hz, 1H); 3.77 (q, J=8.9 Hz, 1H); 3.59 (td, J=5.1, 9.3 Hz, 1H); 2.62-2.55 (overlapped m, 2H).

### Preparation J: 3-(bromoethynyl)-1-(oxetan-3-ylmethyl)azetidine

Starting from the compound of Preparation B (0.505 g; 2.57 mmol) and oxetanone-3-carbaldehyde (0.264 g; 2.91 mmol) and proceeding in analogy to Preparation C, the title compound was obtained, without further purification, as a yellowish oil (0.608 g; >95% yield).

¹H NMR (d6-DMSO) δ: 4.56 (dd, J=5.9, 7.8 Hz, 2H); 4.22-4.19 (m, 2H); 3.42-3.39 (m, 2H); 3.18 (quint, J=7.4 Hz, 1H); 2.94-2.91 (m, 2H); 2.87 (m, 1H); 2.61 (d, J=7.5 Hz, 2H).

### Preparation K: 3-(bromoethynyl)-3-fluoropyrrolidine hydrochloride

### K.i. Tert-butyl 3-(bromoethynyl)-3-hydroxypyrrolidine-1-carboxylate

Starting from (3RS)-tert-butyl 3-ethynyl-3-hydroxypyrrolidine-1-carboxylate (4.49 g; 21.3 mmol) and proceeding in analogy to Preparation B, step B.i, the title compound was obtained as a colourless foam (5.28 g; 86% yield).

¹H NMR (d6-DMSO) δ: 5.94 (s, 1H); 3.44-3.22 (m, 4H); 2.08-1.96 (m, 2H); 1.39 (s, 9H).

MS (ESI, m/z): 289.9 [M+H⁺] for C₁₁H₁₆NO₃B; t_{R}=0.76 min.

### K.ii. Tert-butyl 3-(bromoethynyl)-3-fluoropyrrolidine-1-carboxylate

To a solution of intermediate K.i (2.630 g; 9.06 mmol) in DCM (75 mL), cooled to −78° C., was added (diethylamino)sulfur trifluoride (1.37 mL; 9.34 mmol) dropwise. The reaction proceeded at −78° C. for 30 min and at rt for 45 min. The reaction mixture was poured into cold sat. NaHCO₃ (80 mL). The two layers were separated. The aq. layer was extracted with DCM (2×50 mL). The evaporation residue was purified by CC (Hept-EA) to give the title compound as a yellow oil (1.9 g; 72% yield).

¹H NMR (d6-DMSO) δ: 3.71 (m, 1H); 3.62-3.45 (m, 2H); 3.28 (m, 1H); 2.42-2.23 (m, 2H); 1.40 (s, 9H).

MS (ESI, m/z): 332.89 [M+MeCN+H⁺] for C₁₁H₁₆NO₃B; t_{R}=0.89 min.

### K.iii. 3-(bromoethynyl)-3-fluoropyrrolidine hydrochloride

A solution of intermediate K.ii (1.9 g; 6.5 mmol) in 4MHCl in dioxane (16.5 mL) was stirred at rt for 1 h. The reaction mixture was concentrated to dryness and co-evaporated with Et₂O (20 mL) to give the title compound as a white solid (1.36 g; 92% yield).

¹H NMR (d6-DMSO) δ: 9.78 (s, 1H); 3.72 (m, 1H); 3.57-3.44 (m, 2H); 3.27 (m, 1H); 2.55 (m, 1H); 2.37 (m, 1H).

MS (ESI, m/z): 232.97 [M+MeCN+H⁺] for C₆H₇NBrF; t_{R}=0.22 min.

### Preparation L: 4-(bromoethynyl)-4-fluoro-1-methylpiperidine

### L.i. Tert-butyl 4-(bromoethynyl)-4-fluoropiperidine-1-carboxylate

Starting from tert-butyl 4-ethynyl-4-hydroxypiperidine-1-carboxylate (commercial; 3.04 g; 13.5 mmol) and proceeding successively in analogy to Preparation K, steps K.i and K.ii (75% yield), the title compound was obtained, after purification by CC (Hept-EA), as a yellowish oil (2.34 g).

¹H NMR (d6-DMSO) δ: 3.49-3.35 (m, 4H); 2.01-1.84 (m, 4H); 1.40 (s, 9H).

MS (ESI, m/z): 305.99 [M+H⁺] for C₁₂H₁₇NO₂BrF; t_{R}=0.92 min.

### L.ii. 4-(bromoethynyl)-4-fluoropiperidine trifluoroacetate

To a solution of intermediate L.i (2.34 g; 7.65 mmol) in DCM (40 mL) was added at 0° C. TFA (17.6 mL, 229 mmol). The reaction mixture was stirred at 0° C. for 10 min and at rt for 40 min. The reaction mixture was concentrated to dryness and the resulting oil was triturated in Et₂O.

The resulting solid was dried under HV to afford the title compound as a beige solid (2.42 g; 99% yield).

¹H NMR (d6-DMSO) δ: 8.66 (br. s, 2H); 3.30-3.02 (m, 4H); 2.29-2.04 (m, 4H).

MS (ESI, m/z): 205.99 [M+H⁺] for C₇H₁₀NBrF; t_{R}=0.42 min.

### L.iii. 4-(bromoethynyl)-4-fluoro-1-methylpiperidine

To a solution of intermediate L.ii (0.350 g; 1.09 mmol) in DCM (10 mL) was added 37% aq. formaldehyde (0.244 mL, 3.28 mmol). The reaction mixture was cooled at 0° C. and NaBH(OAc)₃ (1.391 g; 6.56 mmol) was added. The reaction mixture was stirred at 0° C. for 30 min and at rt for 1.25 h. Sat. NaHCO₃ (30 mL) and DCM (20 mL) were added. The aq. layer was extracted with DCM (2×20 mL). The combined org. layers were washed with NaHCO₃ (20 mL), dried over MgSO₄, filtered and concentrated to dryness to afford the title product as a white solid (0.210 g; 83% yield).

¹H NMR (d6-DMSO) δ: 2.42-2.30 (overlapped m, 4H); 2.17 (s, 3H); 1.96-1.89 (m, 4H).

### Preparation M: (2R,3S)-3-(bromoethynyl)-2-methylazetidine

### M.i. Tert-butyl tert-butyl (2S,3S)-1-benzyl-3-((benzyloxy)methyl)azetidine-2-carboxylate

A solution of tert-butyl (S)—N-benzyl-N-(3-(benzyloxy)-2-chloropropyl)glycinate (11 g; 27.2 mmol) in THF (110 mL) and HMPA (11 mL) was cooled to −78° C. and LiHMDS (1M in THF; 41 mL; 41 mmol) was added slowly over a period of 30 min. The mixture allowed to warm 0° C. over a period of 3 h. The reaction was quenched by addition of sat. NH₄Cl (150 mL) and the aq. phase was extracted three times with EA (100 mL). The evaporation residue was purified by CC (Hept-EA) to afford first the (2R,3S)-isomer (5.9 g; 59% yield) and then the (2S,3S)-isomer (2.1 g; 21% yield).

¹H NMR (CDCl₃) δ: 7.37-7.24 (m, 10H); 4.53-4.49 (m, 2H); 3.82 (dd, J=9.2, 6.5 Hz, 1H); 3.78-3.70 (m, 3H); 3.64 (d, J=12.7 Hz, 1H); 3.23 (d, J=6.1 Hz, 1H); 3.03 (t, J=7.4 Hz, 1H); 2.87 (ddt, J=16.8, 10.7, 5.5 Hz, 1H); 1.35 (s, 9H).

MS (ESI, m/z): 368.1 [M+H⁺] for C₂₃H₃₀NO₃; t_{R}=0.78 min.

### M.ii. ((2S,3S)-1-benzyl-3-((benzyloxy)methyl)azetidin-2-yl)methanol

A solution of intermediate M.i (2.0 g; 5.44 mmol) in THF (10 mL) was cooled to 0° C. and a solution of LiAlH₄ (2M in THF, 5.5 mL; 10.9 mmol) was slowly added. The mixture was stirred at 0° C. for 1 h and then warmed to rt. After 2 h the reaction was quenched by careful addition of 1M aq. NaOH (4 mL) and the resulting slurry was stirred for 1 h. The solid was filtered off and the filtrate was concentrated to dryness. The crude product (1.58 g; 93% yield) was used without further purification in the following step.

¹H NMR (CDCl₃) δ: 7.41-7.26 (m, 10H); 4.57-4.52 (m, 2H); 3.69 (d, J=12.6 Hz, 1H); 3.63 (d, J=12.6 Hz, 1H); 3.54 (dd, J=9.5, 5.4 Hz, 1H); 3.51-3.46 (m, 2H); 3.35 (d, J=3.7 Hz, 2H); 3.26 (dt, J=7.3, 3.6 Hz, 1H); 2.89 (br. s, 1H, OH); 2.83 (dd, J=8.5, 6.7 Hz, 1H); 2.75 (m, 1H).

MS (ESI, m/z): 298.2 [M+H⁺] for C₁₉H₂₄NO₂; t_{R}=0.65 min.

### M.iii. ((2S,3S)-1-benzyl-3-((benzyloxy)methyl)azetidin-2-yl)methyl methanesulfonate

A solution of intermediate M.ii (1.4 g; 4.71 mmol) in DCM (30 mL) was cooled to 0° C. and TEA (0.99 mL; 7.06 mmol) was added followed by MsCl (0.44 mL; 5.65 mmol). After 10 min, water (100 mL) was added to the reaction and the two phases were separated. The aq. phase was extracted with DCM (100 mL). The evaporation residue was purified by CC (Hept-EA) to afford the title compound as a colourless oil (1.32 g; 66% yield).

¹H NMR (CDCl₃) δ: 7.38-7.24 (m, 10H); 4.53-4.49 (m, 2H); 4.15-4.09 (m, 2H); 4.09-4.01 (m, 1H); 3.75 (d, J=12.7 Hz, 1H); 3.59 (d, J=12.7 Hz, 1H); 3.50-3.36 (m, 3H); 2.92 (s, 3H); 2.87-2.79 (m, 1H); 2.68-2.61 (m, 1H).

MS (ESI, m/z): 376.0 [M+H⁺] for C₂₀H₂₆NO₄S; t_{R}=0.69 min.

### M.iv. (2R,3S)-1-benzyl-3-((benzyloxy)methyl)-2-methylazetidine

To a solution of intermediate M.iii (1.32 g; 3.52 mmol) in THF (10 mL) at 0° C. was added a solution of LiAlH₄ (2M in THF, 3.5 mL; 7.03 mmol). After 1 h, the mixture was warmed to ambient temperature and stirred for 4 h. The reaction was quenched by careful addition of 1M aq. NaOH (3 mL). The resulting slurry was stirred for 1 h and then filtered. The filtrate was concentrated to dryness and the residue was purified by CC (Hept-EA) to give the title compound as a colourless oil (0.69 g; 70% yield).

¹H NMR (CDCl₃) δ: 7.40-7.24 (m, 10H); 4.54-4.50 (m, 2H); 3.68 (d, J=12.5 Hz, 1H); 3.55-3.49 (m, 4H); 3.06 (q, J=6.6 Hz, 1H); 2.71 (t, J=7.8 Hz, 1H); 2.47-2.39 (m, 1H); 1.11 (d, J=6.1 Hz, 3H).

MS (ESI, m/z): 282.1 [M+H⁺] for C₁₉H₂₄N₂O; t_{R}=0.68 min.

### M.v. Tert-butyl (2R,3S)-3-(hydroxymethyl)-2-methylazetidine-1-carboxylate

A flask was charged with a solution of intermediate M.iv (0.69 g; 2.45 mmol) in MeOH (50 mL) and 10 wt % Pd/C (0.2 g). The mixture was hydrogenated under atmospheric pressure of hydrogen (balloon). After 24 h, the mixture was filtered and the filtrate was concentrated to dryness. The residue was taken up in a THF-H₂O mixture (1-1, 40 mL) and Boc₂O (0.75 g; 3.43 mmol) was added followed by solid NaHCO₃ (0.29 g; 3.43 mmol) and 1M NaOH (10 mL). The solution was stirred for 48 h. The aq. phase was extracted three times with EA (100 mL). The evaporation residue was purified by CC (Hept-EA) to afford the title compound as a colourless oil (0.285 g; 58% yield).

¹H NMR (CDCl₃) δ: 4.05 (p, J=6.1 Hz, 1H); 3.92 (t, J=8.5 Hz, 1H); 3.77 (d, J=6.7 Hz, 2H); 3.60 (dd, J=5.9, 8.7 Hz, 1H); 2.30 (dp, J=6.2, 8.3 Hz, 1H); 1.46 (s, 9H); 1.42 (d, J=6.3 Hz, 3H).

MS (ESI, m/z): 202.2 [M+H⁺] for C₁₀H₁₉NO₃; t_{R}=0.63 min.

### M.vi. (2S,3R)-3-(bromoethynyl)-2-methylazetidine

Starting from the intermediate M.v (0.285 g; 1.42 mmol) and proceeding successively in analogy to Preparation H, steps H.iii to H.v and Preparation I, step I.ii, the title compound was obtained as a yellowish oil (0.08 g; 78% yield).

¹H NMR (d₆-DMSO) δ: 4.05 (m, 1H); 3.65-3.53 (m, 2H); 3.14 (m, 1H); 1.27 (d, J=6.4 Hz, 3H).

### Preparation N: ((2R,3R)-3-(bromoethynyl)azetidin-2-yl)methanol

### N.i. Tert-butyl (R)—N-allyl-N-(3-(benzyloxy)-2-hydroxypropyl)glycinate

A flask was charged with (R)-benzyl glycidyl ether (40.0 g; 244 mmol) and allylamine (183 mL; 2436 mmol). Water (1 mL) was added to the mixture and the reaction was warmed to 55° C. and stirred over night. After removal of the solvent, the crude product was obtained as a yellowish oil (54 g; 100% yield). The latter (54.0 g; 244 mmol) was taken up in THF (500 mL) and tert-butyl bromoacetate (54 mL; 366 mmol) and TEA (68 mL; 488 mmol) were added. The mixture was allowed to stir at rt for 1 h. The reaction mixture was partitioned between water (500 mL) and Et₂O (500 mL). The two phases were separated and the aq. phase was extracted twice with Et₂O (500 mL). The evaporation residue was purified by CC (Hept-EA) to give the product as a colourless oil (68 g; 83% yield).

¹H NMR (CDCl₃) δ: 7.29-7.38 (m, 5H); 5.78-5.89 (m, 1H); 5.14-5.23 (m, 2H); 4.57-4.61 (m, 2H); 3.84-3.91 (m, 1H); 3.73 (s, 1H); 3.51 (m, 2H); 3.33-3.40 (m, 1H); 3.22-3.29 (m, 3H); 2.79-2.84 (m, 1H); 2.56-2.65 (m, 1H); 1.46-1.51 (m, 9H).

MS (ESI, m/z): 336.1 [M+H⁺] for C₁₉H₃₀NO₄; t_{R}=0.71 min.

### N.ii. Tert-butyl (R)—N-allyl-N-(3-(benzyloxy)-2-chloropropyl)glycinate

To a solution of intermediate N.i (68.0 g; 203 mmol) in DCM (500 mL) was added thionyl chloride (30.3 mL; 416 mmol) and the mixture was heated to reflux for 1 h. The mixture was partitioned between DCM (100 mL) and sat. aq. NaHCO₃ (500 mL). The two phases were separated and the aq. phase was extracted with DCM (500 mL). The evaporation residue was taken up in DMF (500 mL) and the mixture was heated to 65° C. for 2 days. The mixture was diluted with water (500 mL) and Et₂O (500 mL) and the phases were separated. The aq. phase was extracted twice with Et₂O (500 mL). The evaporation residue was purified by CC (Hept-EA) to give the product as a colourless oil (60 g; 84% yield).

¹H NMR (CDCl₃) δ: 7.41-7.30 (m, 5H); 5.89-5.73 (m, 1H); 5.26-5.11 (m, 2H); 4.68-4.57 (m, 2H); 4.10 (m, 1H); 3.82-3.77 (m, 1H); 3.72 (m, 1H); 3.40-3.34 (m, 4H); 3.17-3.09 (m, 1H); 3.04-2.90 (m, 1H); 1.51-1.47 (m, 9H).

MS (ESI, m/z): 353.9 [M+H⁺] for C₁₉H₂₉NO₃C₁; t_{R}=0.84 min.

### N.iii. Tert-butyl (2S,3R)-1-allyl-3-((benzyloxy)methyl)azetidine-2-carboxylate and tert-butyl (2R,3R)-1-allyl-3-((benzyloxy)methyl)azetidine-2-carboxylate

A solution of intermediate N.ii (58.7 g; 166 mmol) in THF (600 mL)/HMPA (60 mL) was cooled to −78° C. and a solution of LiHMDS (1M in THF, 250 mL, 250 mmol) was added slowly. The mixture was allowed to warm to 0° C. over 3 h. The reaction was quenched by addition of sat. aq. NH₄Cl. The aq. phase was extracted twice with EA (500 mL). The evaporation residue was purified by CC (Hept-EA) to yield both diastereomers of the product as colourless oils ((2S,3R): 35.3 g; 67% yield; (2R,3R): 7.8 g; 15% yield).

(2S,3R)-Isomer:

¹H NMR (CDCl₃) δ: 7.39-7.29 (m, 5H); 5.92-5.77 (m, 1H); 5.23-5.15 (m, 1H); 5.14-5.06 (m, 1H); 4.55-4.51 (m, 2H); 3.85-3.79 (m, 1H); 3.76-3.69 (m, 1H); 3.67-3.62 (m, 1H); 3.29-3.25 (m, 1H); 3.18-3.12 (m, 2H); 2.97 (t, J=7.4 Hz, 1H); 2.89-2.82 (m, 1H); 1.46-1.41 (m, 9H).

MS (ESI, m/z): 318.1 [M+H⁺] for C₁₉H₂₈NO₃; t_{R}=0.72 min.

(2R,3R)-Isomer:

¹H NMR (CDCl₃) δ: 7.44-7.29 (m, 5H); 5.92-5.79 (m, 1H); 5.26-5.17 (m, 1H); 5.15-5.07 (m, 1H); 4.60-4.54 (m, 2H); 3.62-3.51 (m, 2H); 3.50-3.43 (m, 2H); 3.35-3.26 (m, 1H); 3.08 (m, 1H); 2.90-2.82 (m, 2H); 1.52-1.44 (m, 9H).

MS (ESI, m/z): 318.1 [M+H⁺] for C₁₉H₂₈NO₃; t_{R}=0.72 min.

### N.iv. ((2R,3R)-1-allyl-3-((benzyloxy)methyl)azetidin-2-yl)methanol

Starting from the (2R,3R)-configurated intermediate N.iii (7.8 g; 24.6 mmol) and proceeding in analogy to Preparation M, step M.ii, the title compound was obtained, without purification, as a colourless oil (6 g; >95% yield).

¹H NMR (CDCl₃) δ: 7.42-7.29 (m, 5H); 5.84-5.72 (m, 1H); 5.24-5.18 (m, 1H); 5.15-5.08 (m, 1H); 4.59-4.50 (m, 2H); 3.62-3.56 (m, 1H); 3.55-3.40 (m, 4H); 3.24-3.12 (m, 2H); 3.12-3.03 (m, 1H); 3.03-2.91 (m, 1H); 2.79-2.66 (m, 2H).

MS (ESI, m/z): 248.1 [M+H⁺] for C₁₅H₂₂NO₂; t_{R}=0.57 min.

### N.v. (2R,3R)-1-allyl-3-((benzyloxy)methyl)-2-(((tert-butyldiphenylsilyl)oxy)methyl)-azetidine

To a solution of intermediate N.iv (6 g; 24.3 mmol) in DCM (100 mL) was added TBDPSCl (7.57 mL, 29.1 mmol) and imidazole (2.47 g, 36.4 mmol). The mixture was stirred at rt overnight. The solvent was removed in vacuo and the residue was purified by CC (Hept-EA) to afford the title compound as a colourless oil (11.7 g; >95% yield).

¹H NMR (CDCl₃) δ: 7.73-7.67 (m, 5H); 7.49-7.29 (m, 10H); 5.85-5.70 (m, 1H); 5.21-5.12 (m, 1H); 5.10-5.01 (m, 1H); 4.58-4.44 (m, 2H); 3.86-3.77 (m, 1H); 3.74-3.67 (m, 1H); 3.60-3.44 (m, 3H); 3.39-3.27 (m, 1H); 3.19-3.11 (m, 1H); 3.06-2.94 (m, 1H); 2.78-2.65 (m, 1H); 2.61-2.47 (m, 1H); 1.12-1.03 (m, 9H).

MS (ESI, m/z): 486.2 [M+H⁺] for C₃₁H₃₉NO₂Si; t_{R}=0.94 min.

### N.vi. Tert-butyl (2R,3R)-3-((benzyloxy)methyl)-2-(((tert-butyldiphenylsilyl)oxy)methyl)azetidine-1-carboxylate

To a solution of intermediate N.v (11.7 g; 24.1 mmol) in a DCM-EtOH mixture (1-2; 200 mL) was added N-methyl barbituric acid (5.64 g; 36.1 mmol) and Pd(PPh₃)₄ (1.39 g; 1.2 mmol). The reaction mixture was stirred at rt for 30 min. The solvent was removed in vacuo and the residue was dissolved in DCM (200 mL) and Boc₂O (7.88 g; 36.1 mmol) was added and the mixture was stirred for 18 h. The solvent was removed in vacuo and the evaporation residue was directly subjected to CC (Hept-EA) to afford the title compound as a colourless oil (13.5 g; >95% yield).

¹H NMR (CDCl₃) δ: 7.73-7.65 (m, 4H); 7.48-7.31 (m, 11H); 4.55 (s, 2H); 4.06-3.96 (m, 2H); 3.81-3.72 (m, 1H); 3.69-3.59 (m, 3H); 2.97-2.86 (m, 1H); 2.78-2.72 (m, 1H); 1.40 (s, 9H); 1.08 (s, 9H).

MS (ESI, m/z): 546.1 [M+H⁺] for C₃₃H₄₄NO₄Si; t_{R}=1.16 min.

### N.vii. tert-butyl (2R,3R)-2-(((tert-butyldiphenylsilyl)oxy)methyl)-3-(hydroxymethyl)azetidine-1-carboxylate

To a solution of intermediate N.vi (14 g; 25.7 mmol) in MeOH (200 mL) was added 10 wt % Pd/C (2 g). The mixture was put under a hydrogen atmosphere. After 5 days, the suspension was filtered and the filtrate was concentrated. The evaporation residue was purified by CC (Hept-EA) to afford the title product as a colourless oil (4.45 g; 38% yield) along with reisolated starting material.

¹H NMR (CDCl₃) δ: 7.69 (m, 4H); 7.50-7.36 (m, 6H); 4.11-4.00 (m, 1H); 3.97-3.90 (m, 2H); 3.88-3.84 (m, 1H); 3.84-3.76 (m, 2H); 3.67-3.57 (m, 1H); 2.82-2.69 (m, 1H); 1.39 (s, 9H); 1.14-1.06 (m, 9H).

MS (ESI, m/z): 456.14 [M+H⁺] for C₂₆H₃₇NO₄Si; t_{R}=1.04 min.

### N.viii. ((2R,3R)-3-(bromoethynyl)azetidin-2-yl)methanol

Starting from intermediate N.vii (1.2 g; 2.63 mmol) and proceeding successively in analogy to Preparation H, steps H.iii to H.v and Preparation I, step I.ii, the title compound was obtained as a white solid (0.128 g).

¹H NMR (d6-DMSO) δ: 3.88 (m, 1H); 3.54-3.47 (m, 2H); 3.43 (d, J=4.8 Hz, 2H); 3.39-3.27 (m, 2H); 3.23 (m, 1H).

### Preparation O: 1-(bromoethynyl)-N-methyl cyclopropan-1-amine hydrochloride

### O.i. Tert-butyl (1-(((tert-butyldiphenylsilyl)oxy)methyl)cyclopropyl)carbamate

To a solution of tert-butyl (1-(hydroxymethyl)cyclopropyl)carbamate (3.5 g; 18.7 mmol) and imidazole (2.54 g; 37.4 mmol) in DCM (40 mL) was added TBDPSCl (4.11 mL; 18.7 mmol). The reaction mixture was stirred for 4 h. Water (50 mL) and DCM (20 mL) were added. The two layers were separated and the aq. phase was extracted twice with DCM (2×25 mL). The evaporation residue was purified by CC (EA-Hept) to afford the title compound as a colourless oil (8.85 g; >95% yield).

1H NMR (d6-DMSO) δ: 7.64-7.60 (m, 4H); 7.49-7.40 (m, 6H); 7.20 (s, 1H); 3.66 (s, 2H); 1.36 (br. s, 9H); 1.00 (s, 9H); 0.71-0.65 (m, 2H); 0.64-0.60 (m, 2H).

MS (ESI, m/z): 426.1 [M+H⁺] for C₂₅H₃₅NO₃Si; t_{R}=1.11 min.

### O.ii. Tert-butyl (1-(((tert-butyldiphenylsilyl)oxy)methyl)cyclopropyl) (methyl)carbamate

A suspension of NaH (60% in oil dispersion; 1.33 g; 33.2 mmol) in dry DMF (21 mL) was added dropwise to an ice-chilled solution of intermediate O.i (7.85 g; 18.4 mmol) in dry DMF (13 mL). The reaction mixture was stirred for 30 min then MeI (1.38 mL; 22.1 mmol) was added dropwise. After 3 h stirring at rt, water (200 mL) was added carefully and the resulting suspension was extracted with EA (2×100 mL). The evaporation residue was purified by CC (Hept-EA) to afford the title compound as a white solid (5.78 g; 71% yield).

MS (ESI, m/z): 440.1 [M+H⁺] for C₂₆H₃₇NO₃Si; t_{R}=1.15 min.

### O.iii. 1-(bromoethynyl)-N-methylcyclopropan-1-amine hydrochloride

Starting from the intermediate O.ii (6.57 g; 14.9 mmol), and proceeding successively in analogy to Preparation H, step H.v (97% yield), Preparation H, steps H.iii (91% yield), H.iv (91% yield) and H.v (98% yield), and Preparation K, step K.iii (98% yield), the title compound was obtained, after final trituration in Et₂O, as a white solid (2.4 g).

¹H NMR (d6-DMSO) δ: 9.73 (s, 2H); 2.65 (s, 3H); 1.46-1.42 (m, 2H); 1.29-1.24 (m, 2H).

MS (ESI, m/z): 173.99 [M+H⁺] for C₆H₈NBr; t_{R}=0.35 min.

### Preparation P: ((2R*,4S*)-4-(bromoethynyl)azetidin-2-yl)methanol

### P.i. Tert-butyl (2R,4S)-2,4-bis(hydroxymethyl)azetidine-1-carboxylate

Starting from ((2R*,4S*)-1-benzylazetidine-2,4-diyl)dimethanol (commercial; 5.5 g; 26.5 mmol), and proceeding in analogy to Preparation M, step M.v, the title compound was obtained, after CC (Hept-EA), as a colourless oil (4.47 g; 78% yield).

¹H NMR (CDCl₃) δ: 4.35-4.28 (m, 2H); 3.82-3.78 (m, 2H); 3.71-3.65 (m, 2H); 2.84 (br. s, 2H); 2.21 (m, 1H); 1.93 (m, 1H); 1.49 (s, 9H).

MS (ESI, m/z): 218.1 [M+H⁺] for C₁₀H₁₉NO₄; t_{R}=0.53 min.

### P.ii. Tert-butyl (2S*, 4R)-2-((benzoyloxy)methyl)-4-(hydroxymethyl)azetidine-1-carboxylate

To a solution of intermediate P.i (4.470 g; 20.6 mmol) in THF (500 mL) was added benzoyl chloride (2.39 mL; 20.6 mmol) and TEA (5.75 mL; 41.1 mmol). The mixture was allowed to stir over 3 days. The reaction mixture was partitioned between aq. NaHCO₃ (50 mL) and EA (100 mL). The evaporation residue was purified by CC (Hept-EA) to afford the title compound as a colourless oil (2.5 g, 38% yield).

¹H NMR (CDCl₃) δ: 8.11-8.08 (m, 2H); 7.61 (m, 1H); 7.51-7.47 (m, 2H); 4.58 (m, 1H); 4.49-4.41 (m, 2H); 4.37 (m, 1H); 3.79-3.71 (m, 2H); 2.35 (m, 1H); 2.02 (m, 1H); 1.47 (s, 9H); 1.30 (m, 1H).

MS (ESI, m/z): 322.01 [M+H⁺] for C₁₇H₂₃NO₅; t_{R}=0.82 min.

### P.iii. ((2R*,4S*)-4-(bromoethynyl)azetidin-2-yl)methanol

Starting from intermediate P.ii (2.5 g; 7.78 mmol) and proceeding successively in analogy to Preparation H, steps H.iii to H.vi, and Preparation I, step I.ii, the title compound was obtained as a yellowish foam (0.342 g).

¹H NMR (d6-DMSO) δ: 4.7 (t, J=8.4 Hz, 1H); 4.15-4.06 (m, 2H); 3.48-3.45 (m, 2H); 2.54 (m, 1H); 2.18-2.21 (m, 1H).

### Preparation Q: ((2R,4RS)-4-(bromoethynyl)-1-methylpyrrolidin-2-yl)methanol

### Q.i. Tert-butyl (2R,4R)-2-(((tert-butyldiphenylsilyl)oxy)methyl)-4-((methylsulfonyl)oxy)pyrrolidine-1-carboxylate

To a stirred solution of tert-butyl (2R,4R)-2-(((tert-butyldiphenylsilyl)oxy)methyl)-4-hydroxypyrrolidine-1-carboxylate (prepared as described in WO 2014/078609; 2 g; 4.39 mmol) and TEA (1.22 mL; 8.78 mmol) in DCM (22 mL) at 0° C. was added MsCl (0.35 mL; 4.52 mmol). The reaction mixture was allowed to reach rt over 30 min. Sat. aq. NaHCO₃ (15 mL) was added and the phases were separated. The aq. layer was extracted once with DCM (10 mL). The evaporation residue afforded the crude title compound as a yellow gum (2.37 g; >95% yield).

MS (ESI, m/z): 534.2.0 [M+H⁺] for C₂₇H₃₉NO₆SSi; t_{R}=1.08 min.

### Q.ii. Tert-butyl (2R,4RS)-2-(((tert-butyldiphenylsilyl)oxy)methyl)-4-iodopyrrolidine-1-carboxylate

To a solution of intermediate Q.i (2.37 g, 4.39 mmol) in 2-butanone (17 mL) was added NaI (2 g; 13.4 mmol). The reaction mixture was stirred at 80° C. for 26 h. The reaction mixture was cooled to rt and diluted with water (30 mL) and EA (20 mL). The aq. layer was extracted once with EA (20 mL). The evaporation residue was purified by CC (Hept-EA) to afford the title product as a colourless oil (2.04 g, 81% yield).

MS (ESI, m/z): 566.1 [M+H⁺] for C₂₆H₃₆NO₃IS; t_{R}=1.16 min.

### Q.iii. Tert-butyl (2R,4RS)-2-(((tert-butyldiphenylsilyl)oxy)methyl)-4-((trimethylsilyl)ethynyl)pyrrolidine-1-carboxylate

EtMgBr (1M in THF; 2.65 mL; 2.65 mmol) was added dropwise to a solution of TMS-acetylene (0.38 mL; 2.65 mmol) dissolved in THF (2.7 mL). The mixture was stirred 15 min at rt then 1 h at 50° C. In a separated flask, FeBr₂ (0.06 g, 0.27 mmol) and intermediate Q.ii (1 g; 1.77 mmol) were dissolved in THF (4.5 mL) and NMP (2 mL). The previous warmed Grignard reagent solution was added dropwise over 8 min. The resulting dark mixture was stirred at rt for 3 h. EA (20 mL) and water (15 mL). The two layers were separated. The evaporation residue was purified by CC (Hept-EA) to afford the title compound as an orange gum (0.79 g, 84% yield).

MS (ESI, m/z): 536.2 [M+H⁺] for C₃₁H₄₅NO₃Si₂; t_{R}=1.20 min.

### Q.iv. Tert-butyl (2R,4RS)-2-(((tert-butyldiphenylsilyl)oxy)methyl)-4-ethynylpyrrolidine-1-carboxylate

A solution of intermediate Q.iii (0.71 g; 1.32 mmol) in MeOH (4.5 mL) was treated by K₂CO₃ (0.24 g, 1.72 mmol). The mixture was stirred at rt for 1 h. The reaction was diluted in DCM (50 mL) and water (15 mL). The two layers were separated then the aq. layer was extracted with a DCM-MeOH mixture (9-1; 20 mL). The evaporation residue afforded the crude title product as a yellow oil (0.56 g; 91% yield).

MS (ESI, m/z): 464.2 [M+H⁺] for C₂₈H₃₇NO₃Si; t_{R}=1.13 min.

### Q.v. ((2R,4RS)-4-(bromoethynyl)-1-methylpyrrolidin-2-yl)methanol

Starting from intermediate Q.iv (0.5 g; 1.08 mmol), and proceeding successively as described in Preparation B, steps B.i (81% yield) and B.ii and Preparation L, step L.iii (75% yield over 2 steps), the title compound was obtained, after purification by CC (DCM-MeOH) as a yellowish oil (0.125 g).

¹H NMR (d6-DMSO) δ: 4.44 (m, 1H); 3.36 (m, 1H); 3.22 (m, 1H); 3.12 (dd, J=6.9, 8.3 Hz, 1H); 2.85 (m, 1H); 2.36 (m, 1H); 2.26 (s, 3H); 2.16 (dd, J=8.6, 10.0 Hz, 1H); 1.91 (m, 1H); 1.83 (m, 1H).

MS (ESI, m/z): 218.0 [M+H⁺] for C₈H₁₂NOBr; t_{R}=0.31 min.

### Preparation R: 3-(bromoethynyl)-1-(3-(trityloxy)propyl)azetidine

Starting from the compound of Preparation B (0.640 g; 3.26 mmol) and 3-(trityloxy)propanal (1.57 g; 3.29 mmol) and proceeding as described in Preparation C, the title compound was obtained, after purification by CC (Hept-EA+1% aq. NH₄OH), as a white solid oil (1.0 g; 67% yield).

¹H NMR (d6-DMSO) δ: 7.24-7.39 (m, 15H); 3.37 (t, J=6.8 Hz, 2H); 3.15 (quint, J=7.4 Hz, 1H); 2.97 (t, J=6.4 Hz, 2H); 2.85 (m, 2H); 2.40 (t, J=6.9 Hz, 2H); 1.51 (quint, J=6.7 Hz, 2H).

MS (ESI, m/z): 460.0 [M+H⁺] for C₂₇H₂₆NOBr; t_{R}=0.84 min.

### Preparation S: 3-(2-((di-tert-butoxyphosphoryl)oxy)phenyl)propanoic acid

### S.i. Methyl 3-(2-((di-tert-butoxyphosphoryl)oxy)phenyl)propanoate

To a solution of methyl 3-(2-hydroxyphenyl)propionate (5 g; 30 mmol) in THF (102 mL), cooled at 0° C., was added tetrazole (0.45M in MeCN, 92 mL; 0.042 mol) and di-tert-butyl diisopropylphosphoramidite (12 mL; 36 mmol). The reaction mixture was heated at 40° C. for 24 h. After cooling to 0° C., 30% aq. H₂O₂(22 mL) was added dropwise at 0° C., keeping IT below 10° C. The solution was stirred for 1.5 h at 0° C. Water (200 mL) was added. The aq. layer was extracted with EA (3×100 mL) and the org. layers were washed with 10% aq. NaHSO₃ (100 mL). The evaporation residue was purified by CC (Hept-EA) to afford the title compound as a colourless oil (6.2 g; 60% yield).

¹H NMR (d6-DMSO) δ: 7.35-7.20 (m, 3H); 7.11 (m, 1H); 3.60 (s, 3H); 2.94-2.85 (m, 2H); 2.66-2.56 (m, 2H); 1.45 (s, 18H).

MS (ESI, m/z): 373.0 [M+H⁺] for C₁₈H₂₉O₆P; t_{R}=0.91 min.

### S.ii. 3-(2-((di-tert-butoxyphosphoryl)oxy)phenyl)propanoic acid

To a solution of intermediate S.i (4.3 g; 0.011 mol) in THF-MeOH-water (2-2-1; 100 mL) was added LiOH.H₂O (1.94 g; 46 mmol). The reaction mixture was stirred at rt for 1.5 h. The volatiles were removed in vacuo and the residue was diluted with water (20 mL) and washed with TBME (2×100 mL). This org. layer was discarded. The aq. layer was acidified with 10% aq. citric acid (100 mL) and extracted with EA (3×100 mL). The evaporation residue afforded the title compound as a white solid (3.2 g; 79% yield).

¹H NMR (d6-DMSO) δ: 12.14 (s, 1H); 7.30-7.26 (m, 2H); 7.24 (m, 1H); 7.11 (t, J=7.2 Hz, 1H); 2.88-2.82 (m, 2H); 2.55-2.51 (overlapped m, 2H); 1.45 (s, 18H).

MS (ESI, m/z): 359.0 [M+H⁺] for C₁₇H₂₇O₆P; t_{R}=0.81 min.

### EXAMPLES OF COMPOUNDS ACCORDING TO THE INVENTION

### Example 1: (2R)—N-hydroxy-2-methyl-2-(methyl sulfonyl)-4-(6-((1-(oxetan-3-yl)azetidin-3-yl)buta-1,3-diyn-1-yl)benzo[d]thiazol-2-yl)butanamide

Starting from the compound of Preparation A (0.1 g; 0.23 mmol) and the compound of Preparation C (0.065 g; 0.30 mmol) and proceeding successively in analogy to Procedure A (68% yield) and Procedure B (75% yield), the title compound was obtained, after purification by prep-HPLC (Method 1), as a beige solid (0.057 g).

¹H NMR (d6-DMSO) δ: 11.03 (br. s, 1H); 9.23 (br. s, 1H); 8.35 (m, 1H); 7.97 (d, J=8.5 Hz, 1H); 7.64 (dd, J=1.7, 8.4 Hz, 1H); 4.54 (t, J=6.6 Hz, 2H); 4.33 (dd, J=5.3, 6.4 Hz, 2H); 3.69 (s, 1H); 3.55 (d, J=6.3 Hz, 2H); 3.49 (d, J=7.7 Hz, 1H); 3.20 (overlapped m, 1H); 3.15-3.18 (m, 2H); 3.08 (s, 3H); 2.96 (m, 1H); 2.77 (td, J=4.5, 12.6 Hz, 1H); 2.25 (m, 1H); 1.56 (s, 3H).

MS (ESI, m/z): 488.02 [M+H⁺] for C₂₃H₂₅N₃O₅S₂; t_{R}=0.55 min.

### Example 2: (2R)-5-(2-(4-(hydroxyamino)-3-methyl-3-(methylsulfonyl)-4-oxobutyl)benzo[d]thiazol-6-yl)penta-2,4-diyn-1-yl 4-hydroxypiperidine-1-carboxylate

Starting from the compound of Preparation A (0.1 g; 0.23 mmol) and the compound of Preparation D (0.092 g; 0.30 mmol) and proceeding successively in analogy to Procedure A (68% yield) and Procedure C (56% yield), the title compound was obtained, after purification by prep-HPLC (Method 1), as a beige solid (0.047 g).

¹H NMR (d6-DMSO) δ: 11.18-10.81 (br. s, 1H); 9.44-9.13 (br. s, 1H); 8.40 (d, J=1.4 Hz, 1H); 7.98 (d, J=8.5 Hz, 1H); 7.68 (dd, J=1.4, 8.5 Hz, 1H); 4.89 (s, 2H); 4.78 (d, J=4.0 Hz, 1H); 3.75-3.62 (m, 3H); 3.35-3.23 (overlapped m, 1H); 3.16-3.03 (m, 2H); 3.07 (s, 3H); 3.01-2.93 (m, 1H); 2.83-2.73 (m, 1H); 2.31-2.21 (m, 1H); 1.79-1.66 (m, 2H); 1.56 (s, 3H); 1.36-1.25 (m, 2H).

MS (ESI, m/z): 534.0 [M+H⁺] for C₂₄H₂₇N₃O₇S₂; t_{R}=0.71 min.

### Example 3: (2R)-5-(2-(4-(hydroxyamino)-3-methyl-3-(methylsulfonyl)-4-oxobutyl)benzo[d]thiazol-6-yl)penta-2,4-diyn-1-yl 3-hydroxyazetidine-1-carboxylate

Starting from the compound of Preparation A (0.1 g; 0.23 mmol) and the compound of Preparation E (0.066 g; 0.28 mmol) and proceeding successively in analogy to Procedure A (88% yield) and Procedure C (64% yield), the title compound was obtained, after purification by prep-HPLC (Method 1), as a beige solid (0.067 g).

¹H NMR (d6-DMSO) δ: 11.01 (br. s, 1H); 9.25 (br. s, 1H); 8.39 (d, J=1.4 Hz, 1H); 7.98 (d, J=8.5 Hz, 1H); 7.67 (dd, J=1.4, 8.5 Hz, 1H); 5.74 (d, J=6.6 Hz, 1H); 4.86 (s, 2H); 4.45 (m, 1H); 4.20-4.06 (m, 2H); 3.76-3.64 (m, 2H); 3.27 (m, 1H); 3.07 (s, 3H); 2.96 (m, 1H); 2.76 (m, 1H); 2.25 (m, 1H); 1.55 (s, 3H).

MS (ESI, m/z): 505.9 [M+H⁺] for C₂₂H₂₃N₃O₇S₂; t_{R}=0.68 min.

### Example 4: (2R)—N-hydroxy-2-methyl-4-(6-((1-methylazetidin-3-yl)buta-1,3-diyn-1-yl)benzo[d]thiazol-2-yl)-2-(methylsulfonyl)butanamide

### 4.i. (2R)-4-(6-(azetidin-3-ylbuta-1,3-diyn-1-yl)benzo[d]thiazol-2-yl)-2-methyl-2-(methylsulfonyl)-N-((tetrahydro-2H-pyran-2-yl)oxy)butanamide

Starting from the compound of Preparation A (0.15 g; 0.34 mmol) and the compound of Preparation B (0.094 g; 0.48 mmol) and proceeding in analogy to Procedure A, the title compound was obtained, after purification by CC (DCM-MeOH), as a beige solid (0.126 g; 71% yield).

MS (ESI, m/z): 557.0 [M+MeCN+H⁺] for C₂₅H₂₉N₃O₅S₂; t_{R}=0.64 min.

### 4.ii. (2R)-2-methyl-4-(6-((1-methylazetidin-3-yl)buta-1,3-diyn-1-yl)benzo[d]thiazol-2-yl)-2-(methylsulfonyl)-N-((tetrahydro-2H-pyran-2-yl)oxy) butanamide

To a solution of intermediate 4.i (0.126 g; 0.244 mmol) in DCM (3.19 mL) were added 37% aq. formaldehyde (0.0573 mL; 0.734 mmol) and NaBH(OAc)₃ (0.320 g; 1.47 mmol) The reaction mixture was stirred at rt for 45 min. Sat. aq. NaHCO₃ (10 mL) and DCM (10 mL) were added. The aq. layer was extracted with a DCM-MeOH mixture (9-1; 3×10 mL). The evaporation residue was purified by CC (DCM-MeOH) to afford the title compound as a yellow foam (0.0873 g; 83% yield).

MS (ESI, m/z): 530.1 [M+H⁺] for C₂₆H₂₇N₃O₅S₂; t_{R}=0.65 min.

### 4.iii. (R)—N-hydroxy-2-methyl-4-(6-((1-methylazetidin-3-yl) buta-1,3-diyn-1-yl)benzo[d]thiazol-2-yl)-2-(methylsulfonyl)butanamide

Starting from intermediate 4.ii (0.08 g; 0.16 mmol) and proceeding in analogy to Procedure B, the title compound was obtained, after purification by prep-HPLC (Method 1), as a beige solid (0.038 g; 52% yield).

¹H NMR (d6-DMSO) δ: 11.31-10.26 (br. s, 1H); 8.94-9.58 (br. s, 1H); 8.36 (d, J=1.3 Hz, 1H); 7.97 (d, J=8.5 Hz, 1H); 7.64 (m, 1H); 3.76 (t, J=7.6 Hz, 2H); 3.62-3.51 (m, 1H); 3.39 (t, J=6.9 Hz, 2H); 3.28 (m, 1H); 3.08 (s, 3H); 2.97 (m, 1H); 2.77 (td, J=4.4, 12.6 Hz, 1H); 2.41 (s, 3H); 2.28-2.22 (m, 1H); 1.56 (s, 3H).

MS (ESI, m/z): 487.0 [M+MeCN+H⁺] for C₂₁H₂₃N₃O₄S₂; t_{R}=0.55 min.

### Example 5: (2R)—N-hydroxy-4-(6-((1-(2-hydroxyethyl)azetidin-3-yl)buta-1,3-diyn-1-yl)benzo[d]thiazol-2-yl)-2-methyl-2-(methylsulfonyl)butanamide

### 5.i. (2R)-4-(6-((1-(2-((tert-butyldimethylsilyl)oxy)ethyl)azetidin-3-yl)buta-1,3-diyn-1-yl)benzo[d]thiazol-2-yl)-2-methyl-2-(methylsulfonyl)-N-((tetrahydro-2H-pyran-2-yl)oxy)butanamide

Starting from the compound of Preparation A (0.15 g; 0.34 mmol) and the compound of Preparation F (0.153 g; 0.48 mmol) and proceeding in analogy to Procedure A, the title compound was obtained, after purification by CC (DCM-MeOH), as a beige solid (0.126 g; 71% yield).

MS (ESI, m/z): 674.2 [M+H⁺] for C₃₃H₄₇N₃O₆S₂Si; t_{R}=0.85 min.

### 5.ii. (2R)-4-(6-((1-(2-hydroxyethyl)azetidin-3-yl)buta-1,3-diyn-1-yl)benzo[d]thiazol-2-yl)-2-methyl-2-(methylsulfonyl)-N-((tetrahydro-2H-pyran-2-yl)oxy) butanamide

TBAF (1M in THF; 0.943 mL; 0.943 mmol) was added to a solution of intermediate 5.i (0.158 g; 0.234 mmol) in THF (1 mL). The mixture was stirred at rt for 2 h. The solvent was evaporated and the residue was partitioned between water (150 mL) and EA (200 mL). The two layers were separated. The aq. layer was extracted with EA (2×150 mL). The evaporation residue was purified by CC (DCM-MeOH) to afford the title product as a beige foam (0.076 g; 58% yield).

MS (ESI, m/z): 560.1 [M+H⁺] for C₃₃H₄₇N₃O₆S₂Si; t_{R}=0.54 min.

### 5.iii. (2R)—N-hydroxy-4-(6-((1-(2-hydroxyethyl)azetidin-3-yl)buta-, 3-diyn-1-yl)benzo[d]thiazol-2-yl)-2-methyl-2-(methylsulfonyl) butanamide

Starting from intermediate 5.ii (0.075 g; 0.13 mmol) and proceeding in analogy to Procedure B (64% yield), the title compound was obtained, after purification by prep-HPLC (Method 2), as a beige solid (0.041 g).

¹H NMR (d6-DMSO) δ: 11.03 (m, 1H); 9.26 (d, J=0.8 Hz, 1H); 8.35 (d, J=1.4 Hz, 1H); 7.96 (d, J=8.5 Hz, 1H); 7.64 (dd, J=1.6, 8.4 Hz, 1H); 4.43 (m, 1H); 3.54 (t, J=7.1 Hz, 2H); 3.42 (m, 1H); 3.35 (overlapped m, 2H); 3.27 (m, 1H); 3.10-3.08 (overlapped m, 2H); 3.07 (s, 3H); 2.97 (m, 1H); 2.77 (td, J=4.4, 12.6 Hz, 1H); 2.46 (m, 2H); 2.25 (td, J=5.0, 12.5 Hz, 1H); 1.56 (s, 3H).

MS (ESI, m/z): 476.0 [M+H⁺] for C₂₂H₂₅N₃O₅S₂; t_{R}=0.54 min.

### Example 6: (R)-2-(3-((2-(4-(hydroxyamino)-3-methyl-3-(methylsulfonyl)-4-oxobutyl)benzo[d]thiazol-6-yl)buta-1,3-diyn-1-yl)azetidin-1-yl)ethyl dihydrogen phosphate

Starting from the compound of Preparation A (0.08 g; 0.18 mmol) and the compound of Preparation G (0.154 g; 0.39 mmol) and proceeding successively in analogy to Procedure A (65% yield) and Procedure D (47% yield), the title compound was obtained, after purification by prep-HPLC (Method 1), as a beige solid (0.031 g).

¹H NMR (d6-DMSO) δ: 11.03 (m, 1H); 8.36 (d, J=1.4 Hz, 1H); 7.96 (d, J=8.5 Hz, 1H); 7.65 (dd, J=1.6, 8.4 Hz, 1H); 4.50-3.75 (br. s, 2H); 3.89-3.82 (m, 2H); 3.76-3.70 (m, 2H); 3.62 (m, 1H); 3.54-3.44 (m, 2H); 3.27 (m, 1H); 3.07 (s, 3H); 2.96 (m, 1H); 2.91-2.86 (m, 2H); 2.77 (td, J=4.4, 12.6 Hz, 1H); 2.25 (td, J=5.0, 12.5 Hz, 1H); 1.56 (s, 3H).

MS (ESI, m/z): 556.1.0 [M+H⁺] for C₂₂H₂₆N₃O₈PS₂; t_{R}=0.50 min.

### Example 7: (2R)—N-hydroxy-4-(6-(((1R,2R)-2-(hydroxymethyl)cyclobutyl)buta-1,3-diyn-1-yl)benzo[d]thiazol-2-yl)-2-methyl-2-(methylsulfonyl)butanamide

Starting from the compound of Preparation A (0.075 g; 0.17 mmol) and the compound of Preparation H (0.045 g; 0.24 mmol) and proceeding successively in analogy to Procedure A (>95% yield) and Procedure C (56% yield), the title compound was obtained, after purification by prep-HPLC (Method 1), as a white solid (0.045 g).

30 ¹H NMR (d6-DMSO) δ: 11.00 (br. s, 1H); 9.25 (m, 1H); 8.33 (d, J=1.4 Hz, 1H); 7.96 (d, J=8.5 Hz, 1H); 7.62 (dd, J=1.6, 8.5 Hz, 1H); 4.68 (t, J=5.4 Hz, 1H); 3.43-3.26 (m, 2H); 3.27 (m, 1H); 3.08 (s, 3H); 3.01 (m, 1H); 2.96 (m, 1H); 2.77 (m, 1H); 2.50 (overlapped m, 1H); 2.25 (m, 1H); 2.16 (m, 1H); 1.96 (m, 1H); 1.83 (m, 1H); 1.76 (m, 1H); 1.56 (s, 3H).

MS (ESI, m/z): 461.0 [M+H⁺] for C₂₂H₂₄N₂O₅S₂; t_{R}=0.75 min.

### Example 8: (2R)—N-hydroxy-2-methyl-4-(6-(((2S)-1-methylazetidin-2-yl)buta-1,3-diyn-1-yl)benzo[d]thiazol-2-yl)-2-(methylsulfonyl)butanamide

Starting from the compound of Preparation A (0.12 g; 0.27 mmol) and the compound of Preparation I (0.158 g; 0.98 mmol) and proceeding successively in analogy to Procedure A (54% yield), Example 4, step 4.ii (62% yield) and Procedure B (78% yield), the title compound was obtained, after purification by prep-HPLC (Method 2), as a yellowish solid (0.045 g).

¹H NMR (d6-DMSO) δ: 11.0 (br. s, 1H); 9.25 (br. s, 1H); 8.37 (d, J=1.5 Hz, 1H); 7.97 (d, J=8.5 Hz, 1H); 7.66 (dd, J=1.6, 8.4 Hz, 1H); 3.87 (t, J=7.6 Hz, 1H); 3.31-3.20 (m, 2H); 3.08 (s, 3H); 2.96 (m, 1H); 2.88 (m, 1H); 2.77 (td, J=4.5, 12.5 Hz, 1H); 2.31-2.22 (m, 5H); 2.12 (m, 1H); 1.56 (s, 3H).

MS (ESI, m/z): 445.97 [M+H⁺] for C₂₁H₂₃N₃O₄S₂; t_{R}=0.55 min.

### Example 9: (2R)—N-hydroxy-2-methyl-2-(methyl sulfonyl)-4-(6-((1-(oxetan-3-ylmethyl)azetidin-3-yl)buta-1,3-diyn-1-yl)benzo[d]thiazol-2-yl)butanamide

Starting from the compound of Preparation A (0.080 g; 0.18 mmol) and the compound of Preparation I (0.059 g; 0.25 mmol) and proceeding successively in analogy to Procedure A (84% yield) and Procedure C (58% yield), the title compound was obtained, after purification by prep-HPLC (Method 2), as a white solid (0.045 g).

¹H NMR (d6-DMSO) δ: 11.0 (m, 1H); 9.25 (m, 1H); 8.34 (m, 1H); 7.96 (d, J=8.5 Hz, 1H); 7.64 (m, 1H); 4.59 (dd, J=5.9, 7.8 Hz, 2H); 4.23 (t, J=6.0 Hz, 2H); 3.51-3.46 (m, 2H); 3.40 (m, 1H); 3.27 (m, 1H); 3.08 (s, 3H); 3.05-3.03 (m, 2H); 3.01-2.87 (m, 2H); 2.76 (m, 1H); 2.68-2.64 (m, 2H); 2.24 (m, 1H); 1.56 (s, 3H).

MS (ESI, m/z): 501.9 [M+H⁺] for C₂₄H₂₇N₃O₅S₂; t_{R}=0.55 min.

### Example 10: (2R)—N-hydroxy-4-(6-((1-(3-hydroxypropyl)azetidin-3-yl)buta-1,3-diyn-1-yl)benzo[d]thiazol-2-yl)-2-methyl-2-(methylsulfonyl)butanamide

Starting from intermediate 4.i (0.08 g; 0.15 mmol) and 3-trityloxypropionaldehyde (commercial; 0.13 g; 0.41 mmol) and proceeding successively in analogy to Example 4, step 4.ii (61% yield) and Procedure B (58% yield), the title compound was obtained, after purification by prep-HPLC (Method 2), as a white solid (0.027 g).

¹H NMR (d6-DMSO) δ: 11.0 (br. s, 1H); 9.24 (br. s, 1H); 8.35 (d, J=1.3 Hz, 1H); 7.95 (m, 1H); 7.63 (m, 1H); 4.43-4.36 (m, 1H); 3.50-3.46 (m, 2H); 3.43-3.36 (m, 3H); 3.27 (m, 1H); 3.07 (s, 3H); 3.01-2.93 (m, 3H); 2.76 (m, 1H); 2.42-2.36 (m, 2H); 2.25 (m, 1H); 1.56 (s, 3H); 1.43-1.36 (m, 2H).

MS (ESI, m/z): 490.0 [M+H⁺] for C₂₃H₂₇N₃O₅S; t_{R}=0.54 min.

### Example 11: (2R)-4-(6-((1-cyclopropylazetidin-3-yl)buta-1,3-diyn-1-yl)benzo[d]thiazol-2-yl)-N-hydroxy-2-methyl-2-(methylsulfonyl)butanamide

### 11.i. (2R)-4-(6-((1-cyclopropylazetidin-3-yl) buta-1,3-diyn-1-yl)benzo[d]thiazol-2-yl)-2-methyl-2-(methylsulfonyl)-N-((tetrahydro-2H-pyran-2-yl)oxy)butanamide

To a mixture of intermediate 4.i (0.08 g; 0.155 mmol) and MS 3\acute{Å} (0.2 g) in EtOH (2 mL) were added (1-ethoxycyclopropoxy)trimethylsilane (0.189 mL, 0.931 mmol), NaBH₃CN (0.111 g; 1.76 mmol) and AcOH (0.009 mL; 0.155 mmol). The reaction mixture was stirred at 75° C. for 1 h. After cooling, the solids were removed by filtration and the filtrate was concentrated to dryness. The evaporation residue was purified by CC (DCM-MeOH) to afford the title compound as a white foam (0.025 g; 29% yield).

MS (ESI, m/z): 597.1 [M+MeCN+H⁺] for C₂₈H₃₃N₃O₅S₂; t_{R}=0.69 min.

### 11.ii. (2R)-4-(6-((1-cyclopropylazetidin-3-yl)buta-1,3-diyn-1-yl)benzo[d]thiazol-2-yl)-N-hydroxy-2-methyl-2-(methylsulfonyl) butanamide

Starting from intermediate 11.i (0.025 g; 0.045 mmol) and proceeding in analogy to Procedure B (52% yield), the title compound was obtained, after purification by prep-HPLC (Method 2), as a white solid (0.011 g).

¹H NMR (d6-DMSO) δ: 11.05-11.01 (br. s, 1H); 9.25 (br. s, 1H); 8.35 (d, J=1.2 Hz, 1H); 7.96 (d, J=8.4 Hz, 1H); 7.64 (dd, J=1.6, 8.4 Hz, 1H); 3.53 (t, J=7.4 Hz, 2H); 3.39-3.36 (m, 1H); 3.24 (m, 1H); 3.19-3.16 (m, 2H); 3.07 (s, 3H); 2.96 (m, 1H); 2.77 (m, 1H); 2.28 (m, 1H); 1.86 (m, 1H); 1.56 (s, 3H); 0.35-0.31 (m, 2H); 0.23-0.19 (m, 2H).

MS (ESI, m/z): 513.0 [M+MeCN+H⁺] for C₂₃H₂₅N₃O₄S₂; t_{R}=0.58 min.

### Example 12: (2R)—N-hydroxy-4-(6-((1-(3-hydroxycyclobutyl)azetidin-3-yl)buta-1,3-diyn-1-yl)benzo[d]thiazol-2-yl)-2-methyl-2-(methylsulfonyl)butanamide

Starting from intermediate 4.i (0.08 g; 0.15 mmol) and 3-oxocyclobutyl acetate (0.061 g; 0.465 mmol) and proceeding successively in analogy to Example 4, step 4.ii (82% yield), Preparation H, step H.vi (88% yield) and Procedure B (60% yield), the title compound was obtained, after purification by prep-HPLC (Method 2), as a white solid (0.027 g).

¹H NMR (d6-DMSO) δ: 11.0 (br s, 1H); 9.24 (m, 1H); 8.35 (m, 1H); 7.95 (m, 1H); 7.64 (m, 1H); 4.95 (m, 1H); 4.17 (m, 0.5H); 3.75 (m, 0.5H); 3.50-3.30 (m, 3H); 3.27 (m, 1H); 3.07 (s, 3H); 3.02 (m, 1H); 3.00-2.86 (m, 3H); 2.77 (m, 1H), 2.28-2.16 (m, 2H); 1.93 (m, 1H); 1.76 (m, 1H); 1.60 (m, 1H); 1.56 (overlapped s, 3H).

MS (ESI, m/z): 502.0 [M+H⁺] for C₂₄H₂₇N₃O₅S; t_{R}=0.55 min.

### Example 13: (2R)-4-(6-((1-(2-fluoroethyl)azetidin-3-yl)buta-1,3-diyn-1-yl)benzo[d]thiazol-2-yl)-N-hydroxy-2-methyl-2-(methylsulfonyl)butanamide

To a solution of intermediate 4.i (0.08 g; 0.155 mmol) in DMF (2 mL) and MeOH (2 mL) were added 1-fluoro-2-iodoethane (0.0156 mL, 0.188 mmol) and TEA (0.0264 mL, 0.19 mmol). The reaction mixture was stirred at 80° C. for 2 h. The solvents were removed in vacuo to afford a crude mixture (0.047 g). The latter was treated as described in Procedure B, to afford, after purification by prep-HPLC (Method 2), the title compound as a white solid (0.017 g; 21% yield).

¹H NMR (d6-DMSO) δ: 11.0 (br. s, 1H); 9.25 (br. s, 1H); 8.35 (s, 1H); 7.96 (d, J=8.4 Hz, 1H); 7.65-7.63 (m, 1H); 4.45 (t, J=5.0 Hz, 1H); 4.33 (m, 1H); 3.60-3.53 (m, 2H); 3.45 (m, 1H); 3.16-3.10 (m, 2H); 3.07 (s, 3H); 3.03-2.92 (m, 2H); 2.80-2.60 (m, 3H); 2.29-2.20 (m, 1H); 1.55 (s, 3H).

MS (ESI, m/z): 478.0 [M+H⁺] for C₂₂H₂₄N₃O₄FS₂; t_{R}=0.56 min.

### Example 14: (2R)-4-(6-(((3RS)-3-fluoro-1-methylpyrrolidin-3-yl)buta-1,3-diyn-1-yl)benzo[d]thiazol-2-yl)-N-hydroxy-2-methyl-2-(methylsulfonyl)butanamide

Starting from the compound of Preparation A (0.10 g; 0.23 mmol) and the compound of Preparation K (0.1 g; 0.45 mmol) and proceeding successively in analogy to Procedure A (70% yield), Example 4, step 4.ii (98% yield) and Procedure B (66% yield), the title compound was obtained, after purification by prep-HPLC (Method 2), as a beige solid (0.048 g).

¹H NMR (d6-DMSO) δ: 11.03 (m, 1H); 9.26 (m, 1H); 8.42 (d, J=1.4 Hz, 1H); 8.00 (d, J=8.4 Hz, 1H); 7.70 (dd, J=1.7, 8.5 Hz, 1H); 3.28 (m, 1H); 3.07 (s, 3H); 3.07 (overlapped m, 1H); 2.97 (m, 1H); 2.85-2.74 (m, 3H); 2.50-2.28 (m, 3H); 2.28 (s, 3H); 2.24 (m, 1H); 1.56 (s, 3H).

MS (ESI, m/z): 478.0 [M+H⁺] for C₂₂H₂₄N₃O₄FS₂; t_{R}=0.58 min.

### Example 15: (2R)-4-(6-((4-fluoro-1-methylpiperidin-4-yl)buta-1,3-diyn-1-yl)benzo[d]thiazol-2-yl)-N-hydroxy-2-methyl-2-(methylsulfonyl)butanamide

Starting from the compound of Preparation A (0.07 g; 0.16 mmol) and the compound of Preparation L (0.049 g; 0.22 mmol) and proceeding successively in analogy to Procedure A (97% yield) and Procedure B (62% yield), the title compound was obtained, after purification by prep-HPLC (Method 2), as a beige solid (0.048 g).

¹H NMR (d6-DMSO) δ: 10.95 (br. s, 1H); 9.20 (m, 1H); 8.43 (m, 1H); 7.99 (m, 1H); 7.71 (dd, J=1.4, 8.4 Hz, 1H); 3.28 (m, 1H); 3.07 (s, 3H); 2.97 (m, 1H); 2.78 (m, 1H); 2.46-2.35 (m, 4H); 2.26 (m, 1H); 2.21 (s, 3H); 2.10-1.99 (m, 4H); 1.56 (s, 3H).

MS (ESI, m/z): 533.1 [M+MeCN+H⁺] for C₂₃H₂₆N₃O₄FS₂; t_{R}=0.60 min.

### Example 16: (2R)-4-(6-(((2R,3S)-1,2-dimethylazetidin-3-yl)buta-1,3-diyn-1-yl)benzo[d]thiazol-2-yl)-N-hydroxy-2-methyl-2-(methylsulfonyl)butanamide

Starting from the compound of Preparation A (0.10 g; 0.23 mmol) and the compound of Preparation M (0.056 g; 0.32 mmol) and proceeding successively in analogy to Procedure A (74% yield), Example 4, step 4.ii (82% yield) and Procedure B (61% yield), the title compound was obtained, after purification by prep-HPLC (Method 2), as a beige solid (0.048 g).

¹H NMR (d6-DMSO) δ: 11.03 (br. s, 1H); 9.29-9.23 (br. s, 1H); 8.34 (d, J=1.5 Hz, 1H); 7.96 (m, 1H); 7.63 (m, 1H); 3.56 (m, 1H); 3.27 (m, 1H); 3.07 (s, 3H); 3.01-2.92 (m, 3H); 2.77 (m, 1H); 2.68 (m, 1H); 2.21-2.30 (m, 1H); 2.20 (s, 3H); 1.56 (s, 3H); 1.17 (d, J=5.5 Hz, 3H).

MS (ESI, m/z): 501.1 [M+MeCN+H⁺] for C₂₂H₂₅N₃O₄S₂; t_{R}=0.57 min.

### Example 17: (2R)—N-hydroxy-4-(6-(((2R,3R)-2-(hydroxymethyl)-1-methylazetidin-3-yl)buta-1,3-diyn-1-yl)benzo[d]thiazol-2-yl)-2-methyl-2-(methylsulfonyl)butanamide

Starting from the compound of Preparation A (0.120 g; 0.27 mmol) and the compound of Preparation N (0.103 g; 0.54 mmol) and proceeding successively in analogy to Procedure A (71% yield), Example 4, step 4.ii (80% yield) and Procedure B (68% yield), the title compound was obtained, after purification by prep-HPLC (Method 2), as a beige solid (0.050 g).

¹H NMR (d6-DMSO) δ: 8.34 (d, J=1.1 Hz, 1H); 7.96 (d, J=8.5 Hz, 1H); 7.63 (dd, J=1.4, 8.5 Hz, 1H); 4.70 (t, J=5.6 Hz, 1H); 3.56 (m, 1H); 3.43 (t, J=5.1 Hz, 2H); 3.27 (m, 1H); 3.12 (m, 1H); 3.08 (s, 3H); 3.01-2.93 (m, 2H); 2.80-2.70 (m, 2H); 2.29-2.22 (m, 4H); 1.55 (s, 3H).

MS (ESI, m/z): 476.0 [M+H⁺] for C₂₂H₂₅N₃O₅S₂; t_{R}=0.54 min.

### Example 18: (2R)—N-hydroxy-2-methyl-4-(6-((1-(methylamino)cyclopropyl)buta-1,3-diyn-1-yl)benzo[d]thiazol-2-yl)-2-(methylsulfonyl)butanamide

Starting from the compound of Preparation A (0.100 g; 0.23 mmol) and the compound of Preparation O (0.063 g; 0.3 mmol) and proceeding successively in analogy to Procedure A (80% yield) and Procedure B (57% yield), the title compound was obtained, after filtration, as a beige solid (0.050 g).

¹H NMR (d6-DMSO) δ: 11.03 (s, 1H); 9.26 (s, 1H); 8.36 (d, J=1.4 Hz, 1H); 7.97 (d, J=8.5 Hz, 1H); 7.65 (dd, J=1.6, 8.4 Hz, 1H); 7.20 (m, 1H); 3.27 (m, 1H); 3.08 (s, 3H); 2.97 (m, 1H); 2.77 (td, J=4.5, 12.6 Hz, 1H); 2.49 (s, 3H); 2.25 (m, 1H); 1.56 (m, 3H); 1.15 (s, 4H).

MS (ESI, m/z): 487.0 [M+MeCN+H⁺] for C₂₁H₂₃N₃O₄S₂; t_{R}=0.56 min.

### Example 19: (2R)—N-hydroxy-4-(6-(((2R,3R)-2-(hydroxymethyl)-1-methylazetidin-3-yl)buta-1,3-diyn-1-yl)benzo[d]thiazol-2-yl)-2-methyl-2-(methylsulfonyl)butanamide

Starting from the compound of Preparation A (0.120 g; 0.27 mmol) and the compound of Preparation P (0.150 g; 0.79 mmol) and proceeding successively in analogy to Procedure A (37% yield), Example 4, step 4.ii (66% yield) and Procedure B (68% yield), the title compound was obtained, after purification by prep-HPLC (Method 2), as a yellowish foam (0.017 g).

¹H NMR (d6-DMSO) δ: 11.5 (br. s, 1H); 9.26 (m, 1H); 8.36 (d, J=1.1 Hz, 1H); 7.98 (m, 1H); 7.66 (m, 1H); 4.56 (m, 1H); 3.62 (t, J=8.2 Hz, 1H); 3.37-3.30 (overlapped m, 3H); 3.33-3.20 (overlapped m, 1H); 3.08 (m, 3H); 3.02-2.89 (m, 2H); 2.76 (m, 1H); 2.31 (overlapped s, 3H); 2.27 (m, 1H); 1.88 (m, 1H); 1.59-1.50 (s, 3H) MS (ESI, m/z): 476.0 [M+H⁺] for C₂₂H₂₅N₃O₅S₂; t_{R}=0.53 min.

### Example 20: (2R)—N-hydroxy-4-(6-(((3R,5R)-5-(hydroxymethyl)-1-methylpyrrolidin-3-yl)buta-1,3-diyn-1-yl)benzo[d]thiazol-2-yl)-2-methyl-2-(methylsulfonyl)butanamide

Starting from the compound of Preparation A (0.100 g; 0.23 mmol) and the compound of Preparation Q (0.095 g; 0.43 mmol) and proceeding successively in analogy to Procedure A (61% yield) and Procedure B (75% yield), the title compound was obtained, after purification by prep-HPLC (Method 2), as a yellowish foam (0.051 g).

¹H NMR (d6-DMSO) δ: 11.00 (br. s, 1H); 9.25 (br. s, 1H); 8.34 (d, J=1.6 Hz, 1H); 7.96 (d, J=8.5 Hz, 1H); 7.63 (dd, J=1.6, 8.4 Hz, 1H); 4.49 (t, J=5.5 Hz, 1H); 3.40 (m, 1H); 3.33-3.18 (m, 3H); 3.08 (s, 3H); 2.96 (m, 1H); 2.77 (td, J=4.6, 12.7 Hz, 1H); 2.41 (m, 1H); 2.30 (s, 3H); 2.29-2.21 (m, 3H); 2.03-1.89 (m, 2H); 1.56 (s, 3H).

MS (ESI, m/z): 490.0 [M+H⁺] for C₂₃H₂₇N₃O₅S₂; t_{R}=0.55 min.

### Example 21: (2R)-2-(3-((4-(5-((1-(3-hydroxypropyl)azetidin-3-yl)buta-1,3-diyn-1-yl)benzo[d]thiazol-2-yl)-2-methyl-2-(methylsulfonyl)butanamido)oxy)-3-oxopropyl)phenyl dihydrogen phosphate

### 21.i. (R)—N-hydroxy-2-methyl-2-(methylsulfonyl)-4-(6-((1-(3-(trityloxy)propyl)azetidin-3-yl)buta-1,3-diyn-1-yl)benzo[d]thiazol-2-yl) butanamide

Starting from the compound of Preparation A (0.250 g; 0.57 mmol) and the compound of Preparation R (0.095 g; 0.43 mmol) and proceeding successively in analogy to Procedure A (80% yield) and Procedure C (26% yield), the title compound was obtained, after purification by prep-HPLC (Method 2), as a yellowish foam (0.051 g). The compound of Example 10 (0.075 g) was also isolated.

MS (ESI, m/z): 732.1 [M+H⁺] for C₄₂H₄₁N₃O₅S₂; t_{R}=0.82 min.

### 21.ii. (R)-di-tert-butyl (2-(3-((2-methyl-2-(methylsulfonyl)-4-(5-((1-(3-(trityloxy)propyl)azetidin-3-yl)buta-1,3-diyn-1 yl)benzo[d]thiazol-2-yl)butanamido)oxy)-3-oxopropyl)phenyl)phosphate

To a solution of the compound of Preparation S (0.0523 g; 0.146 mmol) in DMF (2 mL) were added HOBT (0.0281 g; 0.208 mmol), TEA (0.0406 mL; 0.292 mmol), EDC (0.0376 g; 0.194 mmol) and intermediate 21.i (0.083 g; 0.113 mmol). After stirring at rt overnight, the reaction mixture was diluted with EA (25 mL) and aq. NaHCO₃ (25 mL). The evaporation residue was purified by CC (DCM-MeOH+0.5% aq. NH₄OH) to afford the title compound as a yellow foam (0.085 g; 65% yield, 85% purity).

MS (ESI, m/z): [M+H⁺] for C₄₂H₄₁N₃O₅S₂; t_{R}=0.82 min.

### 21.iii. (2R)-2-(3-((4-(5-((1-(3-hydroxypropyl)azetidin-3-yl)buta-, 3-diyn-1-yl)benzo[d]thiazol-2-yl)-2-methyl-2-(methylsulfonyl) butanamido)oxy)-3-oxopropyl)phenyl dihydrogen phosphate

Starting from intermediate 21.ii (0.059 g; 0.055 mmol) and proceeding in analogy to Procedure B, the title compound was obtained, after purification by prep-HPLC (Method 1), as a white solid (0.055 g; 57% yield).

¹H NMR (d6-DMSO) δ: 8.25 (s, 1H); 7.95 (m, 1H); 7.57 (m, 1H); 7.41 (d, J=8.2 Hz, 1H); 7.20 (m, 1H); 7.12 (m, 1H); 6.94 (t, J=7.3 Hz, 1H); 4.09 (m, 2H); 3.92-3.75 (m, 3H); 3.43 (t, J=6.0 Hz, 2H); 3.33-3.26 (m, 3H); 3.18 (m, 1H); 3.14 (s, 3H); 3.06-2.91 (overlapped m, 4H); 2.82-2.62 (overlapped m, 4H); 2.26 (m, 1H); 1.65 (s, 3H); 1.54-1.60 (m, 2H).

MS (ESI, m/z): 717.1 [M+H⁺] for C₃₂H₃₆N₃O₁₀PS₂; t_{R}=0.59 min.

### Pharmacological Properties of the Invention Compounds

### In Vitro Assays

### Bacterial Growth Minimal Inhibitory Concentrations:

### Experimental Methods:

Minimal Inhibitory Concentrations (MICs; mg/L) were determined in cation-adjusted Mueller-Hinton Broth by a microdilution method following the description given in “Methods for Dilution Antimicrobial Susceptibility Tests for Bacteria that Grow Aerobically”, Approved standard, 7^{th} ed., Clinical and Laboratory Standards Institute (CLSI) Document M7-A7, Wayne, Pa., USA (2006).

### Results:

All Example compounds were tested against several Gram-positive and Gram-negative bacteria. Typical antibacterial test results are given in Table 1 hereafter (MICs in mg/L). K. pneumoniae A-651 is a multiply-resistant (in particular quinolone-resistant) strain, while E. coli ATCC25922 and P. aeruginosa ATCC27853 are quinolone-sensitive strains.

TABLE 1

| | MIC for | MIC for | MIC for |
|---|---|---|---|
| Example | E. coli | P. aeruginosa | K. Pneumoniae |
| No. | ATCC25922 | ATCC27853 | A-651 |
| 1 | 0.25 | 1 | 0.25 |
| 2 | 0.25 | 1 | 0.5 |
| 3 | 0.5 | 4 | 1 |
| 4 | 0.25 | 1 | 0.5 |
| 5 | 1 | 1 | 0.5 |
| 7 | 0.125 | 1 | 0.25 |
| 8 | 0.25 | 2 | 1 |
| 9 | 0.125 | 1 | 0.25 |
| 10 | 0.25 | 1 | 0.5 |
| 11 | 0.063 | 2 | 0.125 |
| 12 | 0.125 | 1 | 0.25 |
| 13 | 0.125 | 1 | 0.25 |
| 14 | 0.063 | 1 | 0.25 |
| 15 | 0.25 | 1 | 0.5 |
| 16 | 0.125 | 0.5 | 0.5 |
| 17 | 0.5 | 1 | 1 |
| 18 | 0.125 | 1 | 0.25 |
| 19 | 0.5 | 1 | 2 |
| 20 | 0.5 | 1 | 1 |
| Cipro | ≤0.063 | 0.25 | >32 |

The compounds of Examples 6 and 21 were tested against against wild-type E. coli A-1261 in the absence of alkaline phosphatase or esterase, in the presence of an alkaline phosphatase and in the presence of an esterase. The corresponding antibacterial test results are given in Table 2 hereafter (MICs in mg/L).

TABLE 2

| | Active | MIC for E. coli A-1261 | | |
|---|---|---|---|---|
| | Metabolite | In the absence of | In the presence of an | |
| Example | Example | alkaline phosphatase | alkaline phosphatase | In the presence of an |
| No. | No. | or esterase | (2 i.U./mL) | esterase (10 i.U./mL) |
| 6 | 5 | >16 | 0.5 | >16 |
| 21 | 10 | 0.5 | 0.5 | 8 |

1. A compound of formula I 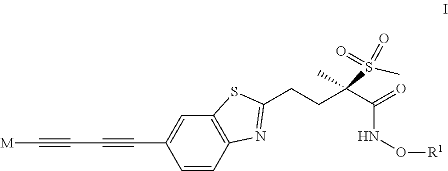
wherein M is the group (4-hydroxypiperidin-1-yl)carbonyloxymethyl, (3-hydroxyazetidin-1-yl)carbonyloxymethyl or 1-(methylamino)cyclopropyl, or M represents one of the groups M^{A}, M^{B}, M^{C}, M^{D}, M^{E} and M^{F} represented below 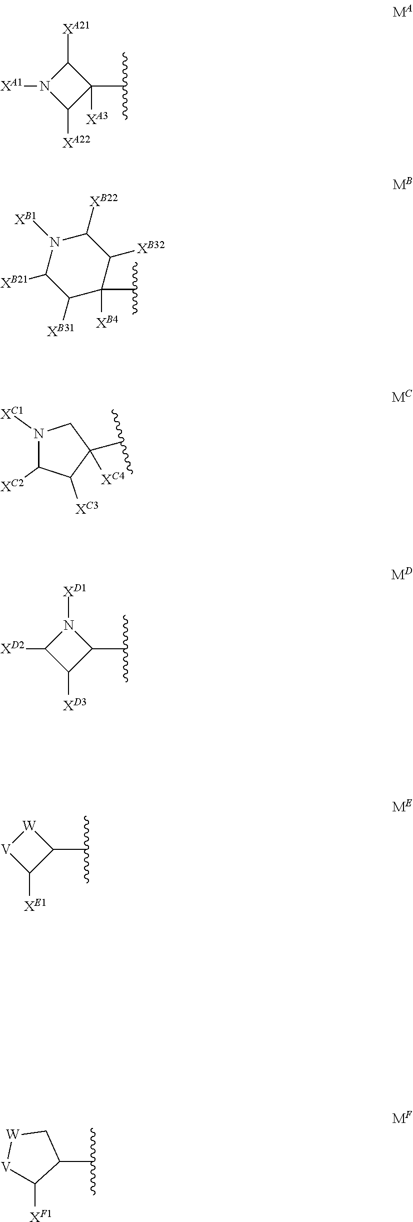
wherein
X^{A1} represents methyl-d, methyl-d2, (C₁-C₄)alkyl, ω-(C₂-C₃)haloalkyl, ω-hydroxy(C₂-C₄)alkyl, 2,3-dihydroxyprop-1-yl, 3-hydroxy-2-(hydroxymethyl)prop-1-yl, oxetan-3-yl, (oxetan-3-yl)methyl, thietan-3-yl, 1,1-dioxidothietan-3-yl, (C₃-C₆)cycloalkyl, 3-hydroxycyclobut-1-yl, 3-(ω-hydroxy(C₁-C₃)alkyl)cyclobut-1-yl, tetrahydropyran-4-yl, (C₃-C₆)cycloalkyl(C₁-C₃)alkyl or ω-phosphonooxy-(C₂-C₄)alkyl;
X^{A21} and X^{A22} each independently represent H, (C₁-C₄)alkyl, (C₁-C₃)haloalkyl or hydroxy(C₁-C₃)alkyl;
X^{A3} represents H, (C₁-C₃)alkyl or halogen;
X^{B1} represents (C₁-C₄)alkyl, ω-hydroxy(C₂-C₃)alkyl, (C₃-C₆)cycloalkyl, oxetan-3-yl or tetrahydropyran-4-yl;
X^{B21} and X^{B22} each independently represent H, (C₁-C₄)alkyl, (C₁-C₃)haloalkyl or hydroxy(C₁-C₃)alkyl;
X^{B31} and X^{B32} each independently represent H, halogen, hydroxy, (C₁-C₄)alkyl, (C₁-C₃)haloalkyl or hydroxy(C₁-C₃)alkyl;
X^{B4} represents H, halogen, hydroxy or (C₁-C₃)alkyl;
X^{C1} represents H, (C₁-C₄)alkyl, (C₃-C₆)cycloalkyl, ω-hydroxy(C₂-C₃)alkyl, oxetan-3-yl or tetrahydropyran-4-yl;
X^{C2} represents H, (C₁-C₄)alkyl, (C₁-C₃)haloalkyl or hydroxy(C₁-C₃)alkyl;
X^{C3} represents H, halogen (especially fluorine), hydroxy, (C₁-C₄)alkyl, (C₁-C₃)haloalkyl or hydroxy(C₁-C₃)alkyl;
X^{C4} represents H, (C₁-C₃)alkyl, halogen or hydroxy;
X^{D1} represents H, (C₁-C₄)alkyl, ω-(C₂-C₃)haloalkyl or ω-hydroxy(C₂-C₄)alkyl;
X^{D2} and X^{D}3 each independently represent H, (C₁-C₄)alkyl, (C₁-C₃)haloalkyl or hydroxy(C₁-C₃)alkyl;
X^{E1} represents H, (C₁-C₄)alkyl, (C₁-C₃)haloalkyl, 1,2-dihydroxyethyl or hydroxy(C₁-C₃)alkyl;
X^{F1} represents H, (C₁-C₄)alkyl, (C₁-C₃)haloalkyl, 1,2-dihydroxyethyl or hydroxy(C₁-C₃)alkyl; and
one of V or W represents —O—, —CH(OH)— or —CH₂—, and the other represents —CH₂—;
R¹ represents H, PO₃H₂, SO₃H, phosphonooxymethyl or the group L represented below 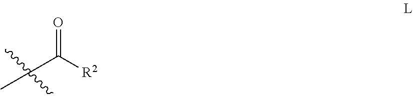
wherein R² represents (C₁-C₄)alkylamino(C₁-C₄)alkyl, [di(C₁-C₄)alkylamino](C₁-C₄)alkyl, phosphonooxy(C₁-C₄)alkyl, phosphonooxymethoxy, 2-(phosphonooxy-(C₁-C₄)alkyl)-phenyl, (2-(phosphonooxy)-phenyl)-(C₁-C₄)alkyl (especially 2-(2-(phosphonooxy)-phenyl)-ethyl) or [2-(phosphonooxy-(C₁-C₄)alkyl)-phenyl]-(C₁-C₄)alkyl;
or a salt thereof.

2. A compound of formula I according to claim 1, which is also a compound of formula I_{CE} 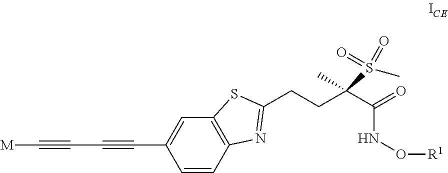
wherein
M is the group (4-hydroxypiperidin-1-yl)carbonyloxymethyl, (3-hydroxyazetidin-1-yl)carbonyloxymethyl or 1-(methylamino)cyclopropyl, or M represents one of the groups M^{A}, M^{B}, M^{C}, M^{D} and M^{E} represented below 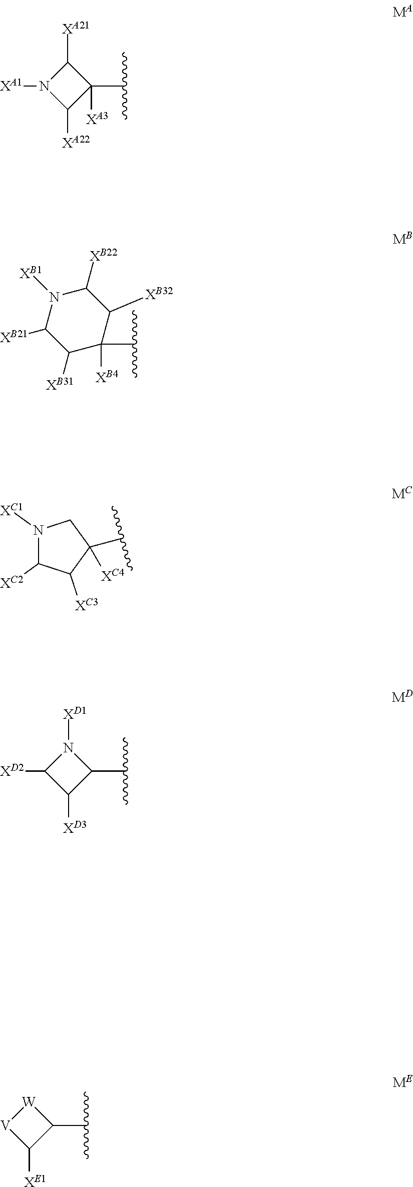
wherein
X^{A1} represents ((C₁-C₄)alkyl, ω-(C₂-C₃)haloalkyl, ω-hydroxy(C₂-C₄)alkyl, oxetan-3-yl, (oxetan-3-yl)methyl, (C₃-C₆)cycloalkyl, 3-hydroxycyclobut-1-yl, or ω-phosphonooxy-(C₂-C₄)alkyl;
one of X^{A21} and X^{A22} represents H and the other represents H, (C₁-C₄)alkyl or hydroxy(C₁-C₃)alkyl;
X^{A3} represents H;
X^{B1} represents (C₁-C₄)alkyl;
each of X^{B21} and X^{B22} represents H;
each of X^{B31} and X^{B32} represents H;
X^{B4} represents halogen;
X^{C1} represents (C₁-C₄)alkyl;
X^{C2} represents H or hydroxy(C₁-C₃)alkyl;
X^{C3} represents H;
X^{C4} represents H or halogen;
X^{D1} represents (C₁-C₄)alkyl;
X^{D2} represents H and X^{D3} represents H or hydroxy(C₁-C₃)alkyl;
X^{E1} represents hydroxy(C₁-C₃)alkyl; and
each of V and W represents —CH₂—;
R¹ represents H or the group L represented below 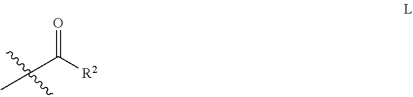
wherein R² represents (2-(phosphonooxy)-phenyl)-(C₁-C₄)alkyl;
or a salt thereof.

3. A compound of formula I according to claim 1, wherein R¹ represents H;
or a salt thereof.

4. A compound of formula I according to claim 1, wherein R¹ does not represent H;
or a salt thereof.

5. A compound of formula I according to claim 1, wherein M is the group (4-hydroxypiperidin-1-yl)carbonyloxymethyl, (3-hydroxyazetidin-1-yl)carbonyloxymethyl or 1-(methylamino)cyclopropyl;
or a salt thereof.

6. A compound of formula I according to claim 1, wherein M is the group M^{A};
or a salt thereof.

7. A compound of formula I according to claim 1, wherein M is the group M^{B};
or a salt thereof.

8. A compound of formula I according to claim 1, wherein M is the group M^{C};
or a salt thereof.

9. A compound of formula I according to claim 1, wherein M is the group M^{D};
or a salt thereof.

10. A compound of formula I according to claim 1, wherein M is the group M^{E};
or a salt thereof.

11. A compound of formula I according to claim 1, wherein R¹ represents H and
M is the group (4-hydroxypiperidin-1-yl)carbonyloxymethyl, (3-hydroxyazetidin-1-yl)carbonyloxymethyl or 1-(methylamino)cyclopropyl; or
M represents the group M^{A} wherein X^{A1} represents ω-hydroxy(C₂-C₄)alkyl, oxetan-3-yl, (oxetan-3-yl)methyl, (C₃-C₆)cycloalkyl, 3-hydroxycyclobut-1-yl; one of X^{A21} and X^{A22} represents H and the other represents H or (C₁-C₄)alkyl; and X^{A3} represents H; or
M represents the group M^{C} wherein X^{C1} represents (C₁-C₄)alkyl; X^{C2} represents H; X^{C3} represents H; and X^{C4} represents halogen;
M represents the group M^{D} wherein X^{D1} represents (C₁-C₄)alkyl; X^{D2} represents H; and X^{D3} represents hydroxy(C₁-C₃)alkyl;
M represents the group M^{E} wherein X^{E1} represents hydroxy(C₁-C₃)alkyl; and
each of V and W represents —CH₂—;
or a salt thereof.

12. A compound of formula I according to claim 1, which is selected from:
(2R)—N-hydroxy-2-methyl-2-(methylsulfonyl)-4-(6-((1-(oxetan-3-yl)azetidin-3-yl)buta-1,3-diyn-1-yl)benzo[d]thiazol-2-yl)butanamide;
(2R)-5-(2-(4-(hydroxyamino)-3-methyl-3-(methylsulfonyl)-4-oxobutyl)benzo[d]thiazol-6-yl)penta-2,4-diyn-1-yl 4-hydroxypiperidine-1-carboxylate;
(2R)-5-(2-(4-(hydroxyamino)-3-methyl-3-(methylsulfonyl)-4-oxobutyl)benzo[d]thiazol-6-yl)penta-2,4-diyn-1-yl 3-hydroxyazetidine-1-carboxylate;
(2R)—N-hydroxy-2-methyl-4-(6-((1-methylazetidin-3-yl)buta-1,3-diyn-1-yl)benzo[d]thiazol-2-yl)-2-(methylsulfonyl)butanamide;
(2R)—N-hydroxy-4-(6-((1-(2-hydroxyethyl)azetidin-3-yl)buta-1,3-diyn-1-yl)benzo[d]thiazol-2-yl)-2-methyl-2-(methylsulfonyl)butanamide;
(R)-2-(3-((2-(4-(hydroxyamino)-3-methyl-3-(methylsulfonyl)-4-oxobutyl)benzo[d]thiazol-6-yl)buta-1,3-diyn-1-yl)azetidin-1-yl)ethyl dihydrogen phosphate;
(2R)—N-hydroxy-4-(6-(((1R,2R)-2-(hydroxymethyl)cyclobutyl)buta-1,3-diyn-1-yl)benzo[d]thiazol-2-yl)-2-methyl-2-(methylsulfonyl)butanamide;
(2R)—N-hydroxy-2-methyl-4-(6-(((2S)-1-methylazetidin-2-yl)buta-1,3-diyn-1-yl)benzo[d]thiazol-2-yl)-2-(methylsulfonyl)butanamide;
(2R)—N-hydroxy-2-methyl-2-(methylsulfonyl)-4-(6-((((1-(oxetan-3-ylmethyl)azetidin-3-yl)buta-1,3-diyn-1-yl)benzo[d]thiazol-2-yl)butanamide;
(2R)—N-hydroxy-4-(6-((1-(3-hydroxypropyl)azetidin-3-yl)buta-1,3-diyn-1-yl)benzo[d]thiazol-2-yl)-2-methyl-2-(methylsulfonyl)butanamide;
(2R)-4-(6-((1-cyclopropylazetidin-3-yl)buta-1,3-diyn-1-yl)benzo[d]thiazol-2-yl)-N-hydroxy-2-methyl-2-(methylsulfonyl)butanamide;
(2R)—N-hydroxy-4-(6-((1-(3-hydroxycyclobutyl)azetidin-3-yl)buta-1,3-diyn-1-yl)benzo[d]thiazol-2-yl)-2-methyl-2-(methylsulfonyl)butanamide;
(2R)-4-(6-((1-(2-fluoroethyl)azetidin-3-yl)buta-1,3-diyn-1-yl)benzo[d]thiazol-2-yl)-N-hydroxy-2-methyl-2-(methylsulfonyl)butanamide;
(2R)-4-(6-(((3-fluoro-1-methylpyrrolidin-3-yl)buta-1,3-diyn-1-yl)benzo[d]thiazol-2-yl)-N-hydroxy-2-methyl-2-(methylsulfonyl)butanamide;
(2R)-4-(6-((4-fluoro-1-methylpiperidin-4-yl)buta-1,3-diyn-1-yl)benzo[d]thiazol-2-yl)-N-hydroxy-2-methyl-2-(methylsulfonyl)butanamide;
(2R)-4-(6-(((2R,3S)-1,2-dimethylazetidin-3-yl)buta-1,3-diyn-1-yl)benzo[d]thiazol-2-yl)-N-hydroxy-2-methyl-2-(methylsulfonyl)butanamide;
(2R)—N-hydroxy-4-(6-(((2R,3R)-2-(hydroxymethyl)-1-methylazetidin-3-yl)buta-1,3-diyn-1-yl)benzo[d]thiazol-2-yl)-2-methyl-2-methylsulfonyl)butanamide;
(2R)—N-hydroxy-2-methyl-4-(6-((1-(methylamino)cyclopropyl)buta-1,3-diyn-1-yl)benzo[d]thiazol-2-yl)-2-(methylsulfonyl)butanamide;
(2R)—N-hydroxy-4-(6-(((2R,3R)-2-(hydroxymethyl)-1-methylazetidin-3-yl)buta-1,3-diyn-1-yl)benzo[d]thiazol-2-yl)-2-methyl-2-methylsulfonyl)butanamide;
(2R)—N-hydroxy-4-(6-(((3R,5R)-5-(hydroxymethyl)-1-methylpyrrolidin-3-yl)buta-1,3-diyn-1-yl)benzo[d]thiazol-2-yl)-2-methyl-2-(methylsulfonyl)butanamide; or
(2R)-2-(3-((4-(5-((1-(3-hydroxypropyl)azetidin-3-yl)buta-1,3-diyn-1-yl)benzo[d]thiazol-2-yl)-2-methyl-2-(methylsulfonyl)butanamido)oxy)-3-oxopropyl)phenyl dihydrogen phosphate;
or a salt thereof.

13. A medicament comprising the compound of formula I as defined in claim 1, or a pharmaceutically acceptable salt thereof.

14. A pharmaceutical composition containing, as active principle, a compound of formula I as defined in claim 1, or a pharmaceutically acceptable salt thereof, and at least one therapeutically inert excipient.

15. A method for the prevention or treatment of a bacterial infection comprising administering the compound of formula I as defined in claim 1, or a pharmaceutically acceptable salt thereof, to a patient in need thereof.
